(19) United States
(12) Patent Application Publication
(10) Pub. No.: US 2012/0237442 A1
(43) Pub. Date: Sep. 20, 2012

(54) Design and Construction of Novel Multivalent Antibodies

(75) Inventors: Edmund A. Rossi, Woodland Park, NJ (US); David M. Goldenberg, Mendham, NJ (US); Chien-Hsing Chang, Downingtown, PA (US)

(73) Assignee: IBC PHARMACEUTICALS, INC., Morris Plains, NJ (US)

(21) Appl. No.: 13/480,650

(22) Filed: May 25, 2012

Related U.S. Application Data
(63) Continuation-in-part of application No. 13/419,614, filed on Mar. 14, 2012.
Application No. 13/419,614 is a division of application No. 12/468,589, filed on May 19, 2009, now Pat. No. 8,163,291.
Application No. 12/468,589 is a division of application No. 11/389,358, filed on Mar. 24, 2006, now Pat. No. 7,550,143.
Application No. 11/389,358 is a continuation-in-part of application No. 13/021,302, filed on Feb. 4, 2011.
Application No. 13/021,302 is a division of application No. 12/417,917, filed on Apr. 3, 2009, now Pat. No. 7,906,121.
Application No. 12/417,917 is a division of application No. 11/478,021, filed on Jun. 29, 2006, now Pat. No. 7,534,866.
Application No. 11/478,021 is a continuation-in-part of application No. 12/968,936, filed on Dec. 15, 2010.
Application No. 12/968,936 is a division of application No. 12/396,965, filed on Mar. 3, 2009, now Pat. No. 7,871,622.
Application No. 12/396,965 is a division of application No. 11/391,584, filed on Mar. 28, 2006, now Pat. No. 7,521,056.
Application No. 11/391,584 is a continuation-in-part of application No. 12/949,536, filed on Nov. 18, 2010, now Pat. No. 8,211,440.
Application No. 12/949,536 is a division of application No. 12/396,605, filed on Mar. 3, 2009, now Pat. No. 7,858,070.
Application No. 12/396,605 is a division of application No. 11/633,729, filed on Dec. 5, 2006, now Pat. No. 7,527,787.
(60) Provisional application No. 61/490,122, filed on May 26, 2011.
(60) Provisional application No. 60/668,603, filed on Apr. 6, 2005.
(60) Provisional application No. 60/728,292, filed on Oct. 19, 2005.
(60) Provisional application No. 60/751,196, filed on Dec. 16, 2005.
(60) Provisional application No. 60/782,332, filed on Mar. 14, 2006.
(60) Provisional application No. 60/864,530, filed on Nov. 6, 2006.

(51) Int. Cl. A61K 39/395; A61P 35/00; A61P 37/00; A61P 37/06; A61P 25/28; A61P 3/00; A61P 9/00; A61P 35/02; A61P 21/04; A61P 7/04; A61P 19/04; A61P 13/12; A61P 17/00; A61P 1/16; A61P 1/02; A61P 19/02; A61P 17/06; A61P 7/06; A61K 51/10; A61P 25/00; C12N 9/96; C07K 19/00; A61P 29/00; A61P 3/10; A61P 5/38; C07K 16/46

(52) U.S. Cl. 424/1.49; 530/387.3; 530/391.1; 530/391.7; 530/391.3; 424/136.1; 424/178.1; 424/183.1; 424/85.1; 424/85.5; 424/85.6; 424/85.7; 424/85.2; 435/188; 530/358; 530/351; 530/359; 530/383; 424/85.4

(57) ABSTRACT

The present invention concerns compositions and use of multivalent and/or multispecific antibodies or immunoconjugates, preferably made by the dock-and-lock technique. The antibodies or immunoconjugates may comprise a first and second polypeptide, each comprising V_{H} and V_{L} domains in series, wherein the first and second polypeptides bind to each other, wherein a V_{H} domain on one polypeptide binds to a complementary V_{L} domain on the other polypeptide to form an antigen binding site, wherein V_{H} and V_{L} domains on the same polypeptide do not bind to each other and wherein one polypeptide is attached to the amino terminal end of a C_{H}1 domain and the other polypeptide is attached to the amino terminal end of a C_{L} domain. The carboxyl terminal end of the C_{H}1 domain may be attached to a C_{H}2-C_{H}3 domain. The antibodies or immunoconjugates are of use to treat a wide variety of diseases.

### RELATED APPLICATIONS

This application claims the benefit of U.S. Provisional Patent Application 61/490,122, filed May 26, 2011. This application is a continuation-in-part of U.S. patent application Ser. No. 13/419,614, filed Mar. 14, 2012, which was a divisional of U.S. patent application Ser. No. 12/468,589 (now issued U.S. Pat. No. 8,163,291), filed May 19, 2009, which was a divisional of U.S. patent application Ser. No. 11/389,358 (now issued U.S. Pat. No. 7,550,143), filed Mar. 24, 2006, which claimed the benefit under 35 U.S.C. 119(e) of Provisional U.S. Patent Applications 60/668,603, filed Apr. 6, 2005, 60/728,292, filed Oct. 19, 2005 and 60/751,196, filed Dec. 16, 2005. This application is a continuation-in-part of U.S. patent application Ser. No. 13/021,302, filed Feb. 4, 2011, which was a divisional of U.S. patent application Ser. No. 12/417,917 (now issued U.S. Pat. No. 7,906,121), filed Apr. 3, 2009, which was a divisional of U.S. patent application Ser. No. 11/478,021 (now issued U.S. Pat. No. 7,534,866), filed Jun. 29, 2006, which claimed the benefit under 35 U.S.C. 119(e) of Provisional U.S. Patent Application 60/782,332, filed Mar. 14, 2006. This application is a continuation-in-part of U.S. patent application Ser. No. 12/968,936, filed Dec. 15, 2010, which was a divisional of U.S. patent application Ser. No. 12/396,965 (now issued U.S. Pat. No. 7,871,622), filed Mar. 3, 2009, which was a divisional of U.S. patent application Ser. No. 11/391,584 (now issued U.S. Pat. No. 7,521,056), filed Mar. 28, 2006. This application is a continuation-in-part of U.S. patent application Ser. No. 12/949,536, filed Nov. 18, 2010, which was a divisional of U.S. patent application Ser. No. 12/396,605 (now issued U.S. Pat. No. 7,858,070), filed Mar. 3, 2009, which was a divisional of U.S. patent application Ser. No. 11/633,729 (now issued U.S. Pat. No. 7,527,787), filed Dec. 5, 2006, which claimed the benefit under 35 U.S.C. 119(e) of Provisional U.S. Patent Application 60/864,530, filed Nov. 6, 2006. The text of each of the priority applications is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on May 22, 2012, is named IBC132US.txt and is 165,257 bytes in size.

### FIELD OF THE INVENTION

The present invention concerns designer fusion modules for building multifunctional, multivalent antibodies and immunoconjugates by the dock-and-lock (DNL) technique. In preferred embodiments, the multivalent antibodies or immunoconjugates may comprise a first and second polypeptide, each comprising V_{H} and V_{L} domains in series, wherein the first and second polypeptides bind to each other to form the antibody complex, wherein a V_{H} domain on one polypeptide binds to a complementary V_{L} domain on the other polypeptide to form an antigen binding site, wherein V_{H} and V_{L} domains on the same polypeptide do not bind to each other and wherein one polypeptide is attached to the amino terminal end of a C_{H}1 domain and the other polypeptide is attached to the amino terminal end of a C_{L} domain. The carboxyl terminal end of the C_{H}1 domain may be attached to a C_{H}2-C_{H}3 domain. In more preferred embodiments, the first and second peptides may have a structure selected from the group consisting of V_{Ha}-V_{Lb}-C_{H}1 and V_{La}-V_{Hb}-C_{L}; V_{Ha}-V_{Lb}-C_{L} and V_{La}-V_{Hb}-C_{H}1; V_{Ha}-V_{Hb}-C_{H}1 and V_{La}-V_{Lb}-C_{L}; V_{Ha}-V_{Hb}-C_{L} and V_{La}-V_{Lb}-C_{H}1; V_{Ha}-V_{Lb}-V_{Hc}-C_{H}1 and V_{La}-V_{Hb}-C_{Lc}-C_{L}; V_{Ha}-V_{Lb}-V_{Hc}-C_{L} and V_{La}-V_{Hb}-V_{Lc}-C_{H}1; V_{Ha}-V_{Hb}-V_{Hc}-C_{H}1 and V_{La}-V_{Lb}-V_{Lc}-C_{L}; V_{Ha}-V_{Hb}-V_{Hc}-C_{L} and V_{La}-V_{Lb}-V_{Lc}-C_{H}1; V_{Ha}-V_{Lb}-V_{Lc}-C_{H}1 and V_{La}-V_{Hb}-V_{Hc}-C_{L}; V_{Ha}-V_{Lb}-V_{Lc}-C_{L} and V_{La}-V_{Hb}-V_{Hc}-C_{H}1; V_{Ha}-V_{Hb}-V_{Lc}-C_{H}1 and V_{La}-V_{Lb}-V_{Hc}-C_{L}; V_{Ha}-V_{Hb}-V_{Lc}-C_{L} and V_{La}-V_{Lb}-V_{Hc}-C_{H}1; VL2-linker-VH1-CH1-Hinge-CH2-CH3 and VH2-linker-VL1-CL; VL1-VH1-X-VH1-CH1-Hinge-CH2-CH3 and VH1-VL1-X-VL1-CL; VL3-VH2-X-VH1-CH1-Hinge-CH2-CH3 and VH3-VL2-X-VL1-CL; and VL2-VH2-X-VH1-CH1 and VH2-VL2-X-VL1-CL. In most preferred embodiments, the first and second polypeptides are fusion proteins further comprising an AD moiety from an AKAP protein or a DDD moiety from human protein kinase A regulatory subunit RIα, RIβ, RIIα or RIIβ, wherein two copies of the DDD moiety form a dimer that binds to the AD moiety to form a DNL complex. The antibodies or immunoconjugates may be conjugated to at least one therapeutic or diagnostic agent. The subject complexes are of use for treating a wide variety of diseases or conditions, such as cancer, autoimmune disease, immune dysregulation disease, organ-graft rejection, graft-versus-host disease, neurodegenerative disease, metabolic disease or cardiovascular disease.

### BACKGROUND

Existing technologies for the production of antibody-based agents having multiple functions or binding specificities suffer a number of limitations. For agents generated by recombinant engineering, such limitations may include high manufacturing cost, low expression yields, instability in serum, instability in solution resulting in formation of aggregates or dissociated subunits, undefined batch composition due to the presence of multiple product forms, contaminating side-products, reduced functional activities or binding affinity/avidity attributed to steric factors or altered conformations, etc. For agents generated by various methods of chemical cross-linking, high manufacturing cost and heterogeneity of the purified product are two major limitations.

In recent years there has been an increased interest in antibodies or other binding moieties that can bind to more than one antigenic determinant (also referred to as epitopes). Generally, naturally occurring antibodies and monoclonal antibodies have two antigen binding sites that recognize the same epitope. In contrast, bifunctional or bispecific antibodies are synthetically or genetically engineered structures that can bind to two distinct epitopes. Thus, the ability to bind to two different antigenic determinants resides in the same molecular construct. Multivalent and/or multispecific antibodies are not limited to only two types of binding sites and may comprise three, four or more different types of binding sites. As used herein, the terms bispecific and multispecific are used interchangeably.

Bispecific antibodies are useful in a number of biomedical applications. For instance, a bispecific antibody with binding sites for a tumor cell surface antigen and for a T-cell surface receptor can direct the lysis of specific tumor cells by T cells. Bispecific antibodies recognizing gliomas and the CD3 epitope on T cells have been successfully used in treating brain tumors in human patients (Nitta, et al. Lancet. 1990; 355:368-371). Numerous methods to produce bispecific antibodies are known. Methods for construction and use of bispecific and multi-specific antibodies are disclosed, for example, in U.S. Pat. No. 7,405,320, the Examples section of which is incorporated herein by reference. Bispecific antibodies can be produced by the quadroma method, which involves the fusion of two different hybridomas, each producing a monoclonal antibody recognizing a different antigenic site (Milstein and Cuello. Nature. 1983; 305:537-540). The fused hybridomas are capable of synthesizing two different heavy chains and two different light chains, which can associate randomly to give a heterogeneous population of 10 different antibody structures of which only one of them, amounting to ⅛ of the total antibody molecules, will be bispecific, and therefore must be further purified from the other forms, which even if feasible will not be cost effective. Furthermore, fused hybridomas are often less stable cytogenetically than the parent hybridomas, making the generation of a production cell line more problematic.

Another method for producing bispecific antibodies uses heterobifunctional cross-linkers to chemically tether two different monoclonal antibodies, so that the resulting hybrid conjugate will bind to two different targets (Staerz, et al. Nature. 1985; 314:628-631; Perez, et al. Nature. 1985; 316:354-356). Bispecific antibodies generated by this approach are essentially heteroconjugates of two IgG molecules, which diffuse slowly into tissues and are rapidly removed from the circulation. Bispecific antibodies can also be produced by reduction of each of two parental monoclonal antibodies to the respective half molecules, which are then mixed and allowed to reoxidize to obtain the hybrid structure (Staerz and Bevan. Proc Natl Acad Sci USA. 1986; 83:1453-1457). An alternative approach involves chemically cross-linking two or three separately purified Fab′ fragments using appropriate linkers. For example, European Patent Application 0453082 (now withdrawn) disclosed the application of a tri-maleimide compound to the production of bi- or tri-specific antibody-like structures. A method for preparing tri- and tetra-valent monospecific antigen-binding proteins by covalently linking three or four Fab fragments to each other via a connecting structure is provided in U.S. Pat. No. 6,511,663. All these chemical methods are undesirable for commercial development due to high manufacturing cost, laborious production process, extensive purification steps, low yields (<20%), and heterogeneous products.

Other methods include improving the efficiency of generating hybrid hybridomas by gene transfer of distinct selectable markers via retrovirus-derived shuttle vectors into respective parental hybridomas, which are fused subsequently (DeMonte, et al. Proc Natl Acad Sci USA. 1990, 87:2941-2945); or transfection of a hybridoma cell line with expression plasmids containing the heavy and light chain genes of a different antibody. These methods also face the inevitable purification problems discussed above.

A method to produce a recombinant bispecific antibody composed of Fab fragments from the same or different antibodies that are brought into association by complementary interactive domains inserted into a region of the antibody heavy chain constant region was disclosed in U.S. Pat. No. 5,582,996. The complementary interactive domains are selected from reciprocal leucine zippers or a pair of peptide segments, one containing a series of positively charged amino acid residues and the other containing a series of negatively charged amino acid residues. One limitation of such a method is that the individual Fab subunits containing the fused complementary interactive domains appear to have much reduced affinity for their target antigens unless both subunits are combined.

Discrete V_{H} and V_{L} domains of antibodies produced by recombinant DNA technology may pair with each other to form a dimer (recombinant Fv fragment) with binding capability (U.S. Pat. No. 4,642,334). However, such non-covalently associated molecules are not sufficiently stable under physiological conditions to have any practical use. Cognate V_{H} and V_{L} domains can be joined with a peptide linker of appropriate composition and length (usually consisting of more than 12 amino acid residues) to form a single-chain Fv (scFv) with binding activity. Methods of manufacturing scFvs are disclosed in U.S. Pat. No. 4,946,778 and U.S. Pat. No. 5,132,405. Reduction of the peptide linker length to less than 12 amino acid residues prevents pairing of V_{H} and V_{L} domains on the same chain and forces pairing of V_{H} and V_{L} domains with complementary domains on other chains, resulting in the formation of functional multimers. Polypeptide chains of V_{H} and V_{L} domains that are joined with linkers between 3 and 12 amino acid residues form predominantly dimers (termed diabodies). With linkers between 0 and 2 amino acid residues, trimers (termed triabody) and tetramers (termed tetrabody) are favored, but the exact patterns of oligomerization appear to depend on the composition as well as the orientation of V-domains (V_{H}-linker-V_{L} or V_{L}-linker-V_{H}), in addition to the linker length.

Monospecific diabodies, triabodies, and tetrabodies with multiple valencies have been obtained using peptide linkers consisting of 5 amino acid residues or less. Bispecific diabodies, which are heterodimers of two different scFvs, each scFv consisting of the V_{H} domain from one antibody connected by a short peptide linker to the V_{L} domain of another antibody, have also been made using a dicistronic expression vector that contains in one cistron a recombinant gene construct comprising V_{H}1-linker-V_{L}2 and in the other cistron a second recombinant gene construct comprising V_{H}2-linker-V_{L}1 (Holliger, et al. Proc Natl Acad Sci USA. 1993; 90: 6444-6448; Atwell, et al. Mol Immunol. 1996; 33:1301-1302; Holliger, et al. Nature Biotechnol. 1997; 15: 632-631; Helfrich, et al. Int. J. Cancer. 1998; 76: 232-239; Kipriyanov, et al. Int J Cancer. 1998; 77: 763-772; Holliger, et al. Cancer Res. 1999; 59: 2909-2916).

A tetravalent tandem diabody (termed tandab) with dual specificity has also been reported (Cochlovius, et al. Cancer Res. 2000; 60: 4336-4341). The bispecific tandab is a dimer of two identical polypeptides, each containing four variable domains of two different antibodies (V_{H}1, V_{L}1, V_{H}2, V_{L}2) linked in an orientation to facilitate the formation of two potential binding sites for each of the two different specificities upon self-association.

To date, the construction of a vector that expresses bispecific or trispecific triabodies has not been achieved. However, polypeptides comprising a collectin neck region are reported to trimerize (Hoppe, et al. FEBS Letters. 1994; 344: 191-195). The production of homotrimers or heterotrimers from fusion proteins containing a neck region of a collectin is disclosed in U.S. Pat. No. 6,190,886.

Methods of manufacturing scFv-based agents of multivalency and multispecificity by varying the linker length were disclosed in U.S. Pat. No. 5,844,094, U.S. Pat. No. 5,837,242 and WO 98/44001. Methods of manufacturing scFv-based agents of multivalency and multispecificity by constructing two polypeptide chains, one comprising of the V_{H} domains from at least two antibodies and the other the corresponding V_{L} domains were disclosed in U.S. Pat. No. 5,989,830 and U.S. Pat. No. 6,239,259. Common problems that have been frequently associated with generating scFv-based agents of multivalency and multispecificity by prior art are low expression levels, heterogeneous products, instability in solution leading to aggregates, instability in serum, and impaired affinity.

A recombinantly produced bispecific or trispecific antibody in which the C-termini of C_{H}1 and C_{L} of a Fab are each fused to a scFv derived from the same or different monoclonal antibodies was disclosed in U.S. Pat. No. 6,809,185. Major deficiencies of this “Tribody” technology include impaired binding affinity of the appended scFvs, heterogeneity of product forms, and instability in solution leading to aggregates.

Thus, there remains a need in the art for a method of making multimeric structures of multiple specificities or functionalities in general, and bispecific antibodies in particular, which are of defined composition, homogeneous purity, and unaltered affinity, and can be produced in high yields without the requirement of extensive purification steps. Furthermore, such structures must also be sufficiently stable in serum to allow in vivo applications. A need exists for stable, multimeric structures of multiple specificities or functionalities that are easy to construct and/or obtain in relatively purified form. Although the discussion above is primarily focused on antibody-containing complexes, the skilled artisan will realize that similar considerations apply to multimeric complexes comprising other types of effector moieties.

### SUMMARY

In various embodiments, the present invention concerns compositions comprising and methods of construction and use of multivalent, multispecific antibodies or antibody-derived binding proteins having two polypeptide chains comprising reciprocal V_{H} and V_{L} domains in series. For example, the design of a trispecific trivalent construct would have a heavy chain polypeptide comprising V_{Ha}-V_{Lb}-V_{Hc} fused to the amino terminal end of the C_{H}1 domain and a light chain polypeptide comprising V_{La}-V_{Hb}-V_{Lc} fused to the amino terminal end of the light chain, where a, b and c represent three different binding specificities. In addition to the preferred arrangement shown above, reciprocal polypeptides could alternatively be arranged as V_{Ha}-V_{Hb}-V_{Hc} & V_{La}-V_{Lb}-V_{Lc}, V_{Ha}-V_{Lb}-V_{Lc} & V_{La}-V_{Hb}-V_{Hc} or V_{Ha}-V_{Hb}-V_{Lc} & V_{La}-V_{Lb}-V_{Hc}; and the reciprocal polypeptide pairs could be alternatively fused to C_{H}1 or C_{L}.

A bivalent construct would have four possible designs:
V_{Ha}-V_{Lb}-C_{H}1 & V_{La}-V_{Hb}-C_{L}
V_{Ha}-V_{Lb}-C_{L} & V_{La}-V_{Hb}-C_{H}1
V_{Ha}-V_{Hb}-C_{H}1 & V_{La}-V_{Lb}-C_{L}
V_{Ha}-V_{Hb}-C_{L} & V_{La}-V_{Lb}-C_{H}1

A trivalent construct has eight possible designs:
V_{Ha}-V_{Lb}-V_{Hc}-C_{H}1 & V_{La}-V_{Hb}-V_{Lc}-C_{L}
V_{Ha}-V_{Lb}-V_{Hc}-C_{L} & V_{La}-V_{Hb}-V_{Lc}-C_{H}1
V_{Ha}-V_{Hb}-V_{Hc}-C_{H}1 & V_{La}-V_{Lb}-V_{Lc}-C_{L}
V_{Ha}-V_{Hb}-V_{Hc}-C_{L} & V_{La}-V_{Lb}-V_{Lc}-CH1
V_{Ha}-V_{Lb}-V_{Lc}-C_{H}1 & V_{La}-V_{Hb}-V_{Hc}-C_{L}
V_{Ha}-V_{Lb}-V_{Lc}-C_{L} & V_{La}-V_{Hb}-V_{Hc}-C_{H}1
V_{Ha}-V_{Hb}-V_{Lc}-C_{H}1 & V_{La}-V_{Lb}-V_{Hc}-C_{L} V_{Ha}-V_{Hb}-V_{Lc}-C_{L} & V_{La}-V_{Lb}-V_{Hc}-C_{H}1
A tetravalent construct has 16 possible designs, and so on.

The order of the variable domains with respect to specificity can be rearranged to optimize functionality. For example, the pair of V_{Ha}-V_{Lb}-V_{Hc}-C_{H}1 & V_{La}-V_{Hb}-V_{Lc}-C_{L} should have the same binding specificities (a, b and c) as V_{Ha}-V_{La}-V_{Hc}-C_{H}1 & V_{Lb}-V_{Ha}-V_{Lc}-C_{L}, but the binding groups (Fv) would have a different spatial relationship, which may or may not affect the binding affinity for each cognate antigen.

In the preferred embodiment, two types of peptide linkers are used to separate the variable domains: a short flexible linker (SH) comprising GGGGS (SEQ ID NO:158) and a rigid hinge linker (HL), both of which should not allow V_{H}-V_{L} pairing within the same polypeptide chain. A polypeptide could employ either or both linkers between variable domains. For example, V_{Ha}-HL-V_{Lb}-HL-V_{Hc}-C_{H}1, V_{Ha}-HL-V_{Lb}-SL-V_{Hc}-C_{H}1, V_{Ha}-SL-V_{Lb}-HL-V_{Hc}-C_{H}1 and V_{H}, SL-V_{Lb}-SL-V_{Hc}-C_{H}1 are all interchangeable formats. Further, any conceived peptide linker of any composition or length could be used instead of these, provided they prohibit intra-chain V_{H}-V_{L} pairing. The IgG1 hinge linker between CH1 and CH2 is EPKSCDKTHTCPPCP (SEQ ID NO:162).

Several potential designs for a construct are shown below.
Tetravalent bispecific IgG: (200 kDa) VL2-linker-VH1-CH1-Hinge-CH2-CH3 (e.g., VL2/VH2 from hLL2, VL1/VH1 from hA20) VH2-linker-VL1-CL
Hexavalent monospecific: (250 kDa) VL1-VH1-X-VH1-CH1-Hinge-CH2-CH3 (e.g., VL1/VH1 from hA20) VH1-VL1-X-VL1-CL
Hexavalent bispecific: (250 kDa) VL2-VH1-X-VH1-CH1-Hinge-CH2-CH3 (e.g. VL2/VH2 from hLL2, VL1/VH1 from hA20) VH2-VL1-X-VL1-CL
Hexavalent trispecific: (250 kDa) VL3-VH2-X-VH1-CH1-Hinge-CH2-CH3 (e.g. VL3/VH3 from anti-CD3, VL2/VH2 from hLL2, VL1/VH1 from hA20) VH3-VL2-X-VL1-CL
Trivalent bispecific: (100 kDa) VL2-VH2-X-VH1-CH1 (e.g., VH2/VL2 from hMN-14, VH1/VL1 from h679) VH2-VL2-X-VL1-CL

In the most basic format, such as those reciprocal polypeptide pairs listed above, the polypeptides will combine to form a multivalent Fab, which may be bivalent and monospecific, bivalent and bispecific, trivalent and monospecific, trivalent and bispecific, trivalent and trispecific, tetravalent and mono, bi-, tri- or tetraspecific, etc. However, the final molecular structure will depend largely on the remainder of the polypeptides beyond the variable domains. If the heavy chain polypeptide comprises the remainder of an IgG heavy chain, a multivalent IgG having two copies of each polypeptide pair will be produced, due to the heterotetrameric quaternary structure of IgG. For example V_{Ha}-V_{Lb}-V_{Hc}-C_{H}1-C_{H}2-C_{H}3 paired with V_{La}-V_{Hb}-V_{Lc}-C_{L} will result in a trispecific hexavalent IgG having two binding groups (Fᵥ) for each specificity. Any modifications that could be made to an IgG could also be made to these structures, including: the addition or deletion of constant region domains; point mutations; and fusion of additional proteins such as cytokines, enzymes or toxins. Specific modifications given as examples herein are the addition of an anchor domain (AD) or dimerization and docking domain (DDD) to convert the fusion protein to a DNL module. The DNL module could then be further enhanced by the conjugation of additional functional groups using the Dock-and-Lock (DNL) method.

In the preferred embodiment, the fusion proteins are produced from a transgene in mammalian cell culture. The fusion proteins could also be produced in eukaryotic systems including myeloma (e.g. Sp2/0 or NS/0), CHO, PerC6, insect or others; or alternatively in prokaryotic systems such as E. coli or P. pastoris.

The variable domains used with this invention could be derived from antibodies of any species (e.g. mouse, rat, rabbit, goat, human, etc) or engineered (e.g. humanized) and having specificity to any given antigen/epitope. For simplicity, only variable domain sequences from three different humanized antibodies are discussed in the Examples below. However, due to the modular design of the antibody constructs, the skilled artisan will realize that any known antibody may be substituted into the disclosed structures.

The antibodies may be incorporated as naked antibodies, alone or in combination with one or more therapeutic agents. Alternatively, the antibodies or fragments thereof may be utilized as immunoconjugates, attached to one or more therapeutic agents. (For methods of making immunoconjugates, see, e.g., U.S. Pat. Nos. 4,699,784; 4,824,659; 5,525,338; 5,677,427; 5,697,902; 5,716,595; 6,071,490; 6,187,284; 6,306,393; 6,548,275; 6,653,104; 6,962,702; 7,033,572; 7,147,856; and 7,259,240, the Examples section of each incorporated herein by reference.) Therapeutic agents may be selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide molecule (e.g., an antisense molecule or a gene) or a second antibody or fragment thereof.

In certain embodiments, a therapeutic and/or diagnostic agent may be administered to a subject after a multivalent, multispecific antibody or antibody-derived binding protein, for example in pre-targeting strategies discussed below. A multivalent, multispecific antibody complex comprising a first antibody against a targeted cell antigen and a second antibody against a hapten may be administered to the subject and allowed to localize in, for example, a diseased tissue such as a tumor. A targetable construct comprising one or more copies of the hapten, along with at least one diagnostic and/or therapeutic agent is subsequently administered and binds to the antibody complex. Where the targetable construct is conjugated to a toxic moiety, such as a radionuclide, this pretargeting method reduces the systemic exposure of the subject to toxicity, allowing a proportionately greater delivery of toxic agent to the targeted tissue.

In some embodiments, the antibody or fragment thereof may be a human, chimeric, or humanized antibody or fragment thereof. A humanized antibody or fragment thereof may comprise the complementarity-determining regions (CDRs) of a murine antibody and the constant and framework (FR) region sequences of a human antibody, which may be substituted with at least one amino acid from corresponding FRs of a murine antibody. A chimeric antibody or fragment thereof may include the light and heavy chain variable regions of a murine antibody, attached to human antibody constant regions. The antibody or fragment thereof may include human constant regions of IgG1, IgG2a, IgG3, or IgG4. Human antibodies may be made by methods known in the art, as discussed below. Exemplary known antibodies of use include, but are not limited to, hR1 (anti-IGF-1R), hPAM4 (anti-mucin), hA20 (anti-CD20), hA19 (anti-CD19), hIMMU31 (anti-AFP), hLL1 (anti-CD74), hLL2 (anti-CD22), hMu-9 (anti-CSAp), hL243 (anti-HLA-DR), hMN-14 (anti-CEACAM5), hMN-15 (anti-CEACAM6), 29H2 (anti-CEACAM1, ABCAM®), hRS7 (anti-EGP-1) and hMN-3 (anti-CEACAM6).

Also disclosed is a method for treating and/or diagnosing a disease or disorder that includes administering to a patient a multivalent, multispecific antibody complex that incorporates or binds to at least one therapeutic and/or diagnostic agent. In preferred embodiments, the disease or disorder may be cancer, hyperplasia, an immune dysregulation disease, an autoimmune disease, organ-graft rejection, graft-versus-host disease, a solid tumor, non-Hodgkin's lymphoma, Hodgkin's lymphoma, multiple myeloma, a B-cell malignancy, a T-cell malignancy, a neurodegenerative disease such as Alzheimer's disease, a metabolic disease such as amyloidosis, diabetes, vasculitis, sepsis, viral infection, fungal infection, bacterial infection, diabetic retinopathy, macular degeneration, asthma, edema, pulmonary hypertension, juvenile diabetes, psoriasis, a cardiovascular disease such as myocardial angiogenesis, plaque neovascularization, restenosis, neointima formation after vascular trauma, angiofibroma, fibrosis associated with chronic inflammation, lung fibrosis, deep venous thrombosis or wound granulation.

A B-cell malignancy may include indolent forms of B-cell lymphomas, aggressive forms of B-cell lymphomas, chronic lymphatic leukemias, acute lymphatic leukemias, and/or multiple myeloma. Solid tumors may include melanomas, carcinomas, sarcomas, and/or gliomas. A carcinoma may include renal carcinoma, lung carcinoma, intestinal carcinoma, stomach carcinoma, breast carcinoma, prostate cancer, ovarian cancer, endometrial cancer, cervical cancer, bladder cancer, liver cancer, pancreatic cancer and/or melanoma.

Antigens that may be targeted by a multivalent, multispecific antibody complex include, but are not limited to, carbonic anhydrase IX, alpha-fetoprotein, α-actinin-4, A3, antigen specific for A33 antibody, ART-4, B7, Ba 733, BAGE, BrE3-antigen, CA125, CAMEL, CAP-1, CASP-8/m, CCCL19, CCCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD45, CD46, CD52, CD54, CD55, CD59, CD64, CD66a-e, CD67, CD70, CD74, CD79a, CD80, CD83, CD95, CD126, CD133, CD138, CD147, CD154, CDC27, CDK-4/m, CDKN2A, CXCR4, colon-specific antigen-p (CSAp), CEA (CEACAM5), CEACAM1, CEACAM6, c-met, DAM, EGFR, EGFRvIII, EGP-1, EGP-2, ELF2-M, Ep-CAM, Flt-1, Flt-3, folate receptor, G250 antigen, GAGE, gp100, GROB, HLA-DR, HM1.24, human chorionic gonadotropin (HCG) and its subunits, HER2/neu, HMGB-1, hypoxia inducible factor (HIF-1), HSP70-2M, HST-2, Ia, IGF-1R, IFN-γ, IFN-α, IFN-β, IL-2, IL-4R, IL-6R, IL-13R, IL-15R, IL-17R, IL-18R, IL-6, IL-8, IL-12, IL-15, IL-17, IL-18, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5, MUM-1/2, MUM-3, NCA66, NCA95, NCA90, antigen specific for PAM-4 antibody, placental growth factor, p53, PLAGL2, prostatic acid phosphatase, PSA, PRAME, PSMA, P1GF, IGF, IGF-1R, IL-6, IL-25, RS5, RANTES, T101, SAGE, 5100, survivin, survivin-2B, TAC, TAG-72, tenascin, TRAIL receptors, TNF-α, Tn antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, Kras, cMET, an oncogene marker and an oncogene product (see, e.g., Sensi et al., Clin Cancer Res 2006, 12:5023-32; Parmiani et al., J Immunol 2007, 178:1975-79; Novellino et al. Cancer Immunol Immunother 2005, 54:187-207). Reports on tumor associated antigens include Mizukami et al., (2005, Nature Med. 11:992-97); Hatfield et al., (2005, Curr. Cancer Drug Targets 5:229-48); Vallbohmer et al. (2005, J. Clin. Oncol. 23:3536-44); and Ren et al. (2005, Ann. Surg. 242:55-63).

Other embodiments may concern methods for treating a lymphoma, leukemia, or autoimmune disorder in a subject, by administering to the subject one or more dosages of a multivalent, multispecific antibody complex, comprising a first binding site against a lymphocyte antigen and a second binding site against the same or a different lymphocyte antigen. The binding site or sites may bind a distinct epitope, or epitopes of an antigen selected from the group consisting of CD4, CD5, CD8, CD14, CD15, CD19, CD20, CD21, CD22, CD23, CD25, CD33, CD37, CD38, CD40, CD40L, CD46, CD52, CD54, CD74, CD80, CD126, CD138, CD154, B7, MUC1, Ia, Ii, HM1.24, HLA-DR, tenascin, VEGF, P1GF, ED-B fibronectin, an oncogene, an oncogene product, NCA 66a-d, necrosis antigens, IL-2, T101, TAG, IL-6, MIF, TRAIL-R1 (DR4) and TRAIL-R2 (DR5). The composition may be parenterally administered in a dosage of 20 to 500 milligrams protein per dose, 20 to 100 milligrams protein per dose, or 20 to 1500 milligrams protein per dose, for example.

Exemplary autoimmune diseases include acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjogren's syndrome, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis, or fibrosing alveolitis.

In still other embodiments, the multivalent, multispecific antibody complexes may be of use to treat subjects infected with pathogenic organisms, such as bacteria, viruses or fungi. Exemplary fungi that may be treated include Microsporum, Trichophyton, Epidermophyton, Sporothrix schenckii, Cryptococcus neoformans, Coccidioides immitis, Histoplasma capsulatum, Blastomyces dermatitidis or Candida albicans. Exemplary viruses include human immunodeficiency virus (HIV), herpes virus, cytomegalovirus, rabies virus, influenza virus, human papilloma virus, hepatitis B virus, hepatitis C virus, Sendai virus, feline leukemia virus, Reo virus, polio virus, human serum parvo-like virus, simian virus 40, respiratory syncytial virus, mouse mammary tumor virus, Varicella-Zoster virus, Dengue virus, rubella virus, measles virus, adenovirus, human T-cell leukemia viruses, Epstein-Barr virus, murine leukemia virus, mumps virus, vesicular stomatitis virus, Sindbis virus, lymphocytic choriomeningitis virus or blue tongue virus. Exemplary bacteria include Bacillus anthracis, Streptococcus agalactiae, Legionella pneumophila, Streptococcus pyogenes, Escherichia coli, Neisseria gonorrhoeae, Neisseria meningitidis, Pneumococcus spp., Hemophilus influenzae B, Treponema pallidum, Lyme disease spirochetes, Pseudomonas aeruginosa, Mycobacterium leprae, Brucella abortus, Mycobacterium tuberculosis or a Mycoplasma. Such multivalent, multispecific antibody complexes may comprise, for example, binding sites for one or more antigenic determinant on a pathogen, and may be conjugated or attached to a therapeutic agent for the pathogen, for example an anti-viral, antibiotic or anti-fungal agent. Alternatively, a multivalent, multispecific antibody complex may comprise a first binding site for a pathogen antigen and a second binding site for a hapten or carrier that is attached to one or more therapeutic agents.

Various embodiments may concern methods of treating inflammatory and immune-dysregulatory diseases, infectious diseases, pathologic angiogenesis or cancer. The multivalent, multispecific antibody complexes may bind to two different targets selected from the group consisting of (A) proinflammatory effectors of the innate immune system, (B) coagulation factors, (C) complement factors and complement regulatory proteins, and (D) targets specifically associated with an inflammatory or immune-dysregulatory disorder or with a pathologic angiogenesis or cancer, wherein the latter target is not (A), (B), or (C). At least one of the targets is (A), (B) or (C). Suitable combinations of targets are described in U.S. patent application Ser. No. 11/296,432, filed Dec. 8, 2005, the Examples section of which is incorporated herein in their entirety.

The proinflammatory effector of the innate immune system to which the multivalent, multispecific antibody complex may bind may be a proinflammatory effector cytokine, a proinflammatory effector chemokine or a proinflammatory effector receptor. Suitable proinflammatory effector cytokines include MIF, HMGB-1 (high mobility group box protein 1), TNF-a, IL-1, IL-4, IL-5, IL-6, IL-8, IL-12, IL-15, and IL-18. Examples of proinflammatory effector chemokines include CCL19, CCL21, IL-8, MCP-1, RANTES, MIP-1A, MIP-1B, ENA-78, MCP-1, IP-10, GROB, and Eotaxin. Proinflammatory effector receptors include IL-4R (interleukin-4 receptor), IL-6R (interleukin-6 receptor), IL-13R (interleukin-13 receptor), IL-15R (interleukin-15 receptor) and IL-18R (interleukin-18 receptor).

The multivalent, multispecific antibody complex also may react specifically with at least one coagulation factor, particularly tissue factor (TF) or thrombin. In other embodiments, the multivalent, multispecific antibody complex reacts specifically with at least one complement factor or complement regulatory protein. In preferred embodiments, the complement factor is selected from the group consisting of C3, C5, C3a, C3b, and C5a. When the Multivalent, multispecific antibody complex reacts specifically with a complement regulatory protein, the complement regulatory protein preferably is selected from the group consisting of CD46, CD55, CD59 and mCRP.

Also described herein are nucleic acids comprising DNA sequences encoding a fusion protein or other subunit of a multivalent, multispecific antibody complex, as described herein. Other embodiments concern expression vectors and/or host cells comprising the encoding DNA sequences. In certain preferred embodiments, the host cell may be an Sp2/0 cell line transformed with a mutant Bcl-2 gene, for example with a triple mutant Bcl-2 gene (T69E, S70E, S87E), that has been adapted to cell transformation and growth in serum free medium. (See, e.g., U.S. Pat. Nos. 7,531,327; 7,537,930; and 7,608,425, the Examples section of each of which is incorporated herein by reference.) The host cell transfected with expression vector(s) encoding a multivalent, multispecific antibody complex, or a subunit thereof, may be cultured by standard techniques for production of the encoded protein or complex. Advantageously, the host cell is adapted for growth and protein production under serum-free conditions.

Another embodiment concerns methods of delivering a diagnostic or therapeutic agent, or a combination thereof, to a target comprising (i) providing a composition that comprises a multivalent, multispecific antibody complex conjugated to at least one diagnostic and/or therapeutic agent and (ii) administering to a subject in need thereof the conjugated multivalent, multispecific antibody complex, wherein the complex comprises at least one antibody or antigen-binding fragment thereof that binds to a targeted cell antigen.

Also contemplated is a method of delivering a diagnostic agent, a therapeutic agent, or a combination thereof to a target, comprising: (a) administering to a subject a multivalent, multispecific antibody complex having an affinity toward a targeted cell antigen and a second affinity toward one or more haptens; (b) waiting a sufficient amount of time for antibody complex that does not bind to the target cell to clear the subject's blood stream; and (c) administering to said subject a carrier molecule comprising a diagnostic agent, a therapeutic agent, or a combination thereof, that binds to the antibody complex.

The skilled artisan will realize that the multivalent, multispecific antibody complexes and uses thereof disclosed above are exemplary only and that many other different types of antibody complexes, for either therapeutic or diagnostic use, are included within the scope of the present invention.

### BRIEF DESCRIPTION OF THE DRAWINGS

FIG. 1. Basic elements of the DNL technique. (a) DDD-module of precursor A. (b) DDD-mediated dimer of precursor A. (c) AD-module of precursor B. (d) Stably tethered DNL conjugate comprising two copies of precursor A and one precursor B. Cysteine residues of DDD and AD are shown as rings; “locking” disulfide bonds are shown as interlocking rings.

FIG. 2. Fab-based DNL-modules and Tri-Fabs (TF). (a) C_{H}1-DDD1-Fab, a Fab-DDD-module without locking cysteine residues in the DDD, which is fused to the carboxyl-terminal end of the Fd chain. (b) N-DDD2-Fab, a Fab-DDD-module with added cysteine residues in the DDD, which is fused to the amino-terminal end of the Fd chain. (c) C_{H}1-DDD2-Fab, a Fab-DDD-module with cysteine residues in the DDD, which is fused to the carboxyl-terminal end of the Fd chain. (d) C_{H}1-AD1-Fab, a Fab-AD-module without locking cysteine residues in the AD, which is fused to the carboxyl-terminal end of the Fd chain. (e) C_{H}1-AD2-Fab, a Fab-AD-module with cysteine residues in the AD, which is fused to the carboxyl-terminal end of the Fd chain. (f) binary, non-covalent complex of C_{H}1-DDD1-Fab-hMN-14 and C_{H}1-AD1-Fab-h679 (g) TF1, a covalent complex of N-DDD2-Fab-hMN-14 and C_{H}1-AD2-Fab-h679 (h) TF2, a covalent complex of C_{H}1-DDD2-Fab-hMN-14 and C_{H}1-AD2-Fab-h679. Variable (V, black or white) and constant (C, grey) domains of the heavy (H) and light (L) chains are represented as ovals. The DDD and AD peptides are shown as helices with the locations indicated for the reactive sulfhydryl groups (SH) of the engineered cysteine residues in AD2 and DDD2, and the disulfide bridges (S) that they form in TF1 and TF2.

FIG. 3. DNL modules for multivalent antibodies. (a) C_{H}3-AD2-IgG, an IgG-AD2 module having two AD2 peptides. (b) bsHexAb, comprising an IgG and two stabilized Fab dimers. (c) C_{H}1-(AD2)₂-Fab, a Fab module comprising tandem AD2 helices separated by a short peptide linker. (d) bispecific antibody comprising five Fabs. (e) C_{H}3-AD2-DVD-IgG, a duel variable domain IgG-AD2 module, with binding specificities “a” and “b”, and possessing two AD2 peptides. (f) a trispecific octavalent antibody comprising a dual variable domain IgG, having binding specificities “a” and “b”, and two stabilized Fab dimers of binding specificity “c”. Variable (V, black, white or light grey) and constant (C, dark grey) domains of the heavy (H) and light (L) chains are represented as ovals. The DDD2 and AD2 peptides are shown as helices with the locations indicated for the reactive sulfhydryl groups (SH) and disulfide bridges (S).

FIG. 4. Cytokine modules and immunocytokines. (a) IFNα-DDD2, a dimeric module comprising DDD2 fused to the amino-terminal end of IFNα. (b) DDD2-G-CSF, a dimeric module comprising DDD2 fused to the carboxyl-terminal end of G-CSF. (c) IgG-2b-2b (e.g. 20-2b-2b), an immunocytokine comprising an IgG and four IFNα groups. (d) IgG-(Fab)₂-2b (e.g. 20-C2-2b), a bispecific immunocytokine comprising an IgG, a stabilized Fab dimer and two IFNα groups.

FIG. 5. Diagrams depicting the expression cassettes (Top) and expressed protein (Bottom) for C2/20-IgG, a bs2Fv-IgG.

FIG. 6. Diagrams depicting the expression cassettes (Top) and expressed protein (Bottom) for 20/20/20-IgG, a 4Fv-IgG.

FIG. 7. Diagrams depicting the expression cassettes (Top) and expressed protein (Bottom) for 20/C2/20-IgG, a bs4Fv-IgG.

FIG. 8. Diagrams depicting the expression cassettes (Top) and expressed protein (Bottom) for 3/C2/20-IgG, a ts4Fv-IgG.

FIG. 9. Diagrams depicting the expression cassettes (Top) and expressed protein (Bottom) for 3/C2/20-IgG-alt#1, a ts4Fv-IgG.

FIG. 10. Diagrams depicting the expression cassettes (Top) and expressed protein (Bottom) for 3/C2/20-IgG-alt#2, a ts4Fv-IgG.

FIG. 11. Diagrams depicting the expression cassettes (Top) and expressed protein (Bottom) for 3/C2/20-IgG-alt#3, a ts4Fv-IgG.

FIG. 12. Diagrams depicting the expression cassettes (Top) and expressed protein (Bottom) for 20/3/20-Fab, a bs2Fv-Fab.

FIG. 13. Diagrams depicting the expression cassettes (Top) and expressed protein (Bottom) for C2/20-IgG-AD2.

FIG. 14. Diagrams depicting the expression cassettes (Top) and expressed protein (Bottom) for C2/20-Fab-DDD2.

FIG. 15. Diagrams depicting the expression cassettes (Top) and expressed protein (Bottom) for 20/C2-IgG-AD2.

### ABBREVIATIONS

V_{H}, heavy chain variable domain of an immunoglobulin.

V_{L}, light chain variable domain of an immunoglobulin (kappa or lambda).

V_{K}, kappa light chain variable domain of an immunoglobulin.

C_{H}1, C_{H}2, C_{H}3, heavy chain constant domains 1, 2 and 3 of an immunoglobulin.

C_{K}, kappa light chain constant domain.

Fᵥ, binding unit composed of a V_{H} and corresponding V_{L}.

HL, hinge linker (EFPKPSTPPGSSGGA, SEQ ID NO:157) derived from hinge region of murine IgG3.

SL, short linker (GGGGS, SEQ ID NO:158).

FL, flexible linker (GSGGGGSGG, SEQ ID NO:159).

2Fv-IgG, tetravalent antibody comprising two Fᵥs and an IgG.

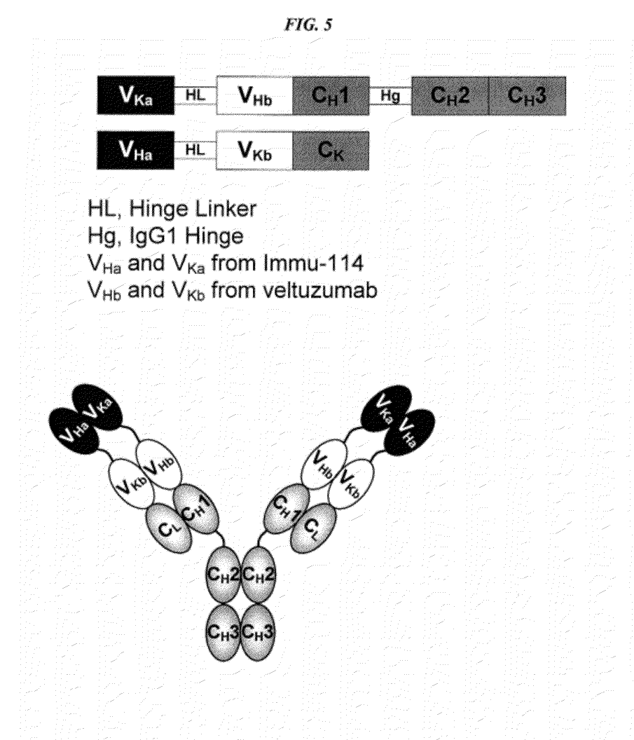

bs2Fv-IgG, bispecific version of 2Fv-IgG (FIG. 5).

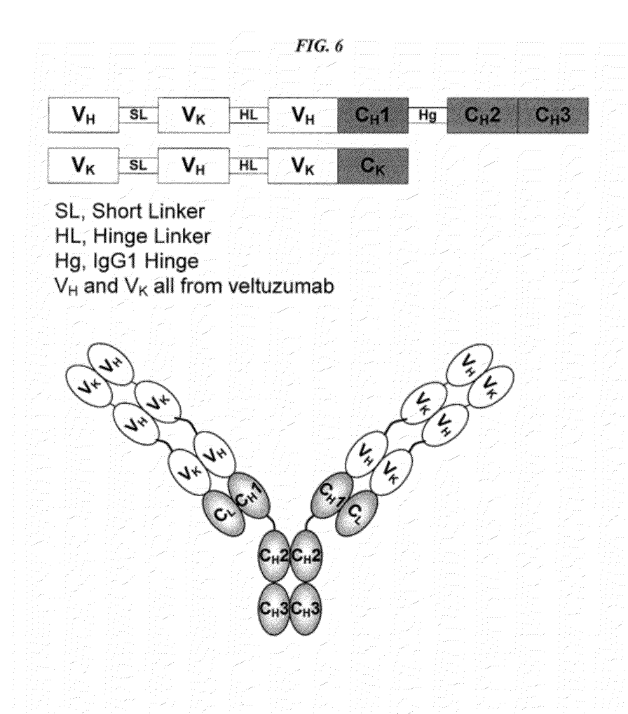

4Fv-IgG, hexavalent antibody comprising four Fᵥs and an IgG (FIG. 6).

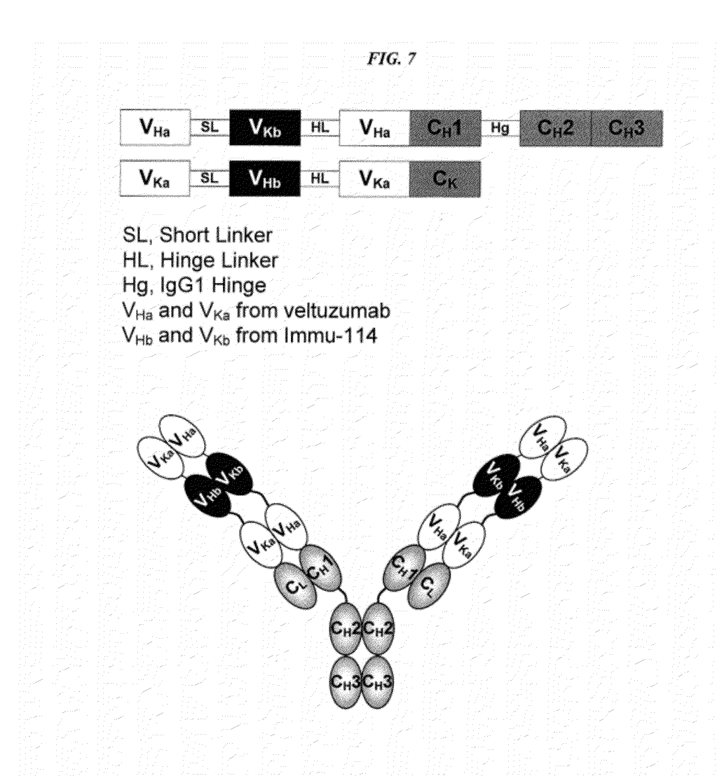

bs4Fv-IgG, bispecific version of 4Fv-IgG (FIG. 7).

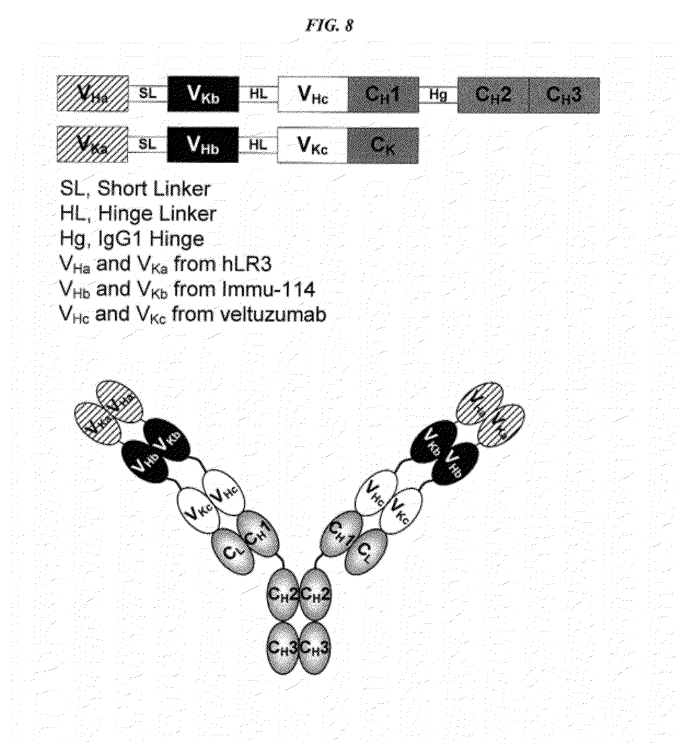

ts4Fv-IgG, trispecific version of 4Fv-IgG, four designs (FIG. 7-8).

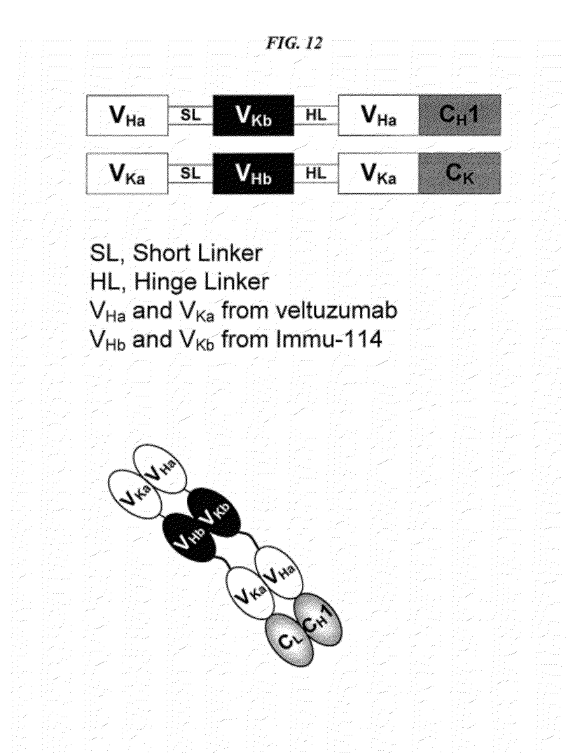

2Fv-Fab, trivalent antibody comprising two Fᵥs and one Fab (FIG. 12).

bs2Fv-Fab, bispecific version of 2Fv-Fab (FIG. 12)

ts2Fv-Fab, trispecific version of 2Fv-Fab

AD, anchor domain derived from an A-kinase anchoring protein.

DDD, dimerization and docking domain derived from protein A kinase.

DNL, Dock-and-Lock Method.

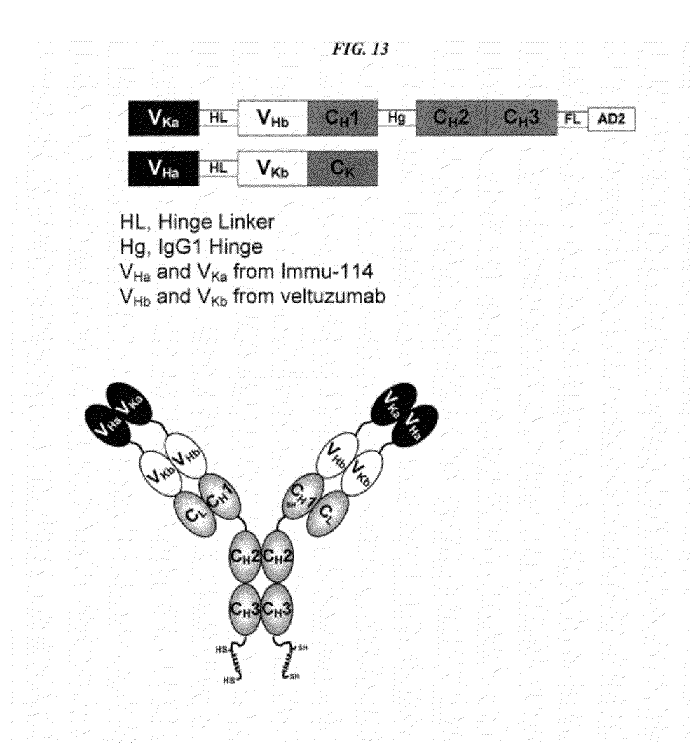

2Fv-IgG-AD2, AD2-module of 2Fv-IgG (FIG. 13).

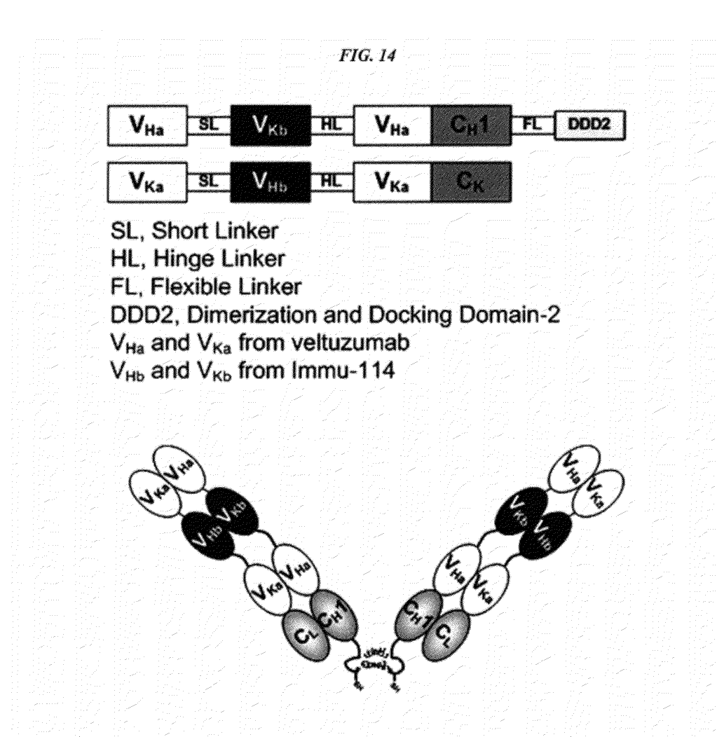

2Fv-Fab-DDD2, DDD2-module of 2Fv-Fab (FIG. 14).

MTX, methotrexate.

ELISA, enzyme linked immunosorbant assay.

Exemplary Antibodies

veltuzumab, a.k.a. hA20, humanized anti-human CD20 IgG1.

Immu-114, a.k.a. hL243γ4p, humanized anti-HLA-DR IgG4.

hLR3, humanized anti-human CD3 IgG1.

### Codes

20 denotes hA20

C2 denotes hL243 (1 mm-114)

3 denotes hLR3

Examples of Constructs (F₁, is italicized).

C2/20-IgG denotes a bs2Fv-IgG comprising two Fᵥs of hL243 and one IgG of hA20.

C2/20-IgG-AD2 denotes the AD2-module of C2/20-IgG.

20/20/20-IgG denotes a 4Fv-IgG comprising four Fᵥs of hA20 and one IgG of hA20.

20/C2/20-IgG denotes a bs4Fv-IgG comprising two Fᵥs of hA20, two Fᵥs of hL243 and one IgG of hA20

3/C2/20-IgG denotes a ts4Fv-IgG comprising two Fᵥs of hLR3, two Fᵥs of hL243 and one IgG of hA20.

20/3/20-Fab denotes a bs2Fv-Fab comprising one Fᵥ of hA20, one Fᵥ of hLR3 and one Fab of hA20.

20/3/20-Fab-DDD2 denotes a DDD2-module of 20/3/20. C2/3/20-Fab denotes a ts2Fv-Fab comprising one Fv of hL243, one Fv of hLR3, and one Fab of hA20.

C2/3/20-Fab-DDD2 denotes a DDD2-module of C213120-Fab.

### DEFINITIONS

As used herein, the terms “a”, “an” and “the” may refer to either the singular or plural, unless the context otherwise makes clear that only the singular is meant.

An “antibody” refers to a full-length (i.e., naturally occurring or formed by normal immunoglobulin gene fragment recombinatorial processes) immunoglobulin molecule (e.g., an IgG antibody) or an immunologically active, antigen-binding portion of an immunoglobulin molecule, like an antibody fragment.

An “antibody fragment” is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” also includes isolated fragments consisting of the variable regions, such as the “Fv” fragments consisting of the variable regions of the heavy and light chains and recombinant single chain polypeptide molecules in which light and heavy variable regions are connected by a peptide linker (“scFv proteins”). As used herein, the term “antibody fragment” does not include portions of antibodies without antigen binding activity, such as Fc fragments or single amino acid residues.

The term “antibody fusion protein” may refer to a recombinantly produced antigen-binding molecule in which one or more of the same or different single-chain antibody or antibody fragment segments with the same or different specificities are linked. Valency of the fusion protein indicates how many binding arms or sites the fusion protein has to a single antigen or epitope; i.e., monovalent, bivalent, trivalent or multivalent. The multivalency of the antibody fusion protein means that it can take advantage of multiple interactions in binding to an antigen, thus increasing the avidity of binding to the antigen. Specificity indicates how many antigens or epitopes an antibody fusion protein is able to bind; i.e., monospecific, bispecific, trispecific, multispecific. Using these definitions, a natural antibody, e.g., an IgG, is bivalent because it has two binding arms but is monospecific because it binds to one epitope. Monospecific, multivalent fusion proteins have more than one binding site for an epitope but only bind with one epitope. The fusion protein may comprise a single antibody component, a multivalent or multispecific combination of different antibody components or multiple copies of the same antibody component. The fusion protein may additionally comprise an antibody or an antibody fragment and a therapeutic agent. Examples of therapeutic agents suitable for such fusion proteins include immunomodulators and toxins. However, the term is not limiting and a variety of protein or peptide effectors may be incorporated into a fusion protein. In another non-limiting example, a fusion protein may comprise an AD or DDD sequence for producing a DNL construct as discussed below.

A “chimeric antibody” is a recombinant protein that contains the variable domains including the complementarity determining regions (CDRs) of an antibody derived from one species, preferably a rodent antibody, while the constant domains of the antibody molecule are derived from those of a human antibody. For veterinary applications, the constant domains of the chimeric antibody may be derived from that of other species, such as a cat or dog.

A “humanized antibody” is a recombinant protein in which the CDRs from an antibody from one species; e.g., a rodent antibody, are transferred from the heavy and light variable chains of the rodent antibody into human heavy and light variable domains. Additional FR amino acid substitutions from the parent, e.g. murine, antibody may be made. The constant domains of the antibody molecule are derived from those of a human antibody.

A “human antibody” is an antibody obtained from transgenic mice that have been genetically engineered to produce human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A fully human antibody also can be constructed by genetic or chromosomal transfection methods, as well as phage display technology, all of which are known in the art. (See, e.g., McCafferty et al., Nature 348:552-553 (1990) for the production of human antibodies and fragments thereof in vitro, from immunoglobulin variable domain gene repertoires from unimmunized donors). In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see, e.g. Johnson and Chiswell, Current Opinion in Structural Biology 3:5564-571 (1993). Human antibodies may also be generated by in vitro activated B cells. (See, U.S. Pat. Nos. 5,567,610 and 5,229,275).

A “therapeutic agent” is an atom, molecule, or compound that is useful in the treatment of a disease. Examples of therapeutic agents include but are not limited to antibodies, antibody fragments, drugs, toxins, enzymes, nucleases, hormones, immunomodulators, antisense oligonucleotides, chelators, boron compounds, photoactive agents, dyes and radioisotopes.

A “diagnostic agent” is an atom, molecule, or compound that is useful in diagnosing a disease. Useful diagnostic agents include, but are not limited to, radioisotopes, dyes, contrast agents, fluorescent compounds or molecules and enhancing agents (e.g., paramagnetic ions). Preferably, the diagnostic agents are selected from the group consisting of radioisotopes, enhancing agents, and fluorescent compounds.

Antibodies and Antibody Fragments

The skilled artisan will realize that the antibodies or fragments thereof discussed herein are exemplary and that antibodies or fragments thereof against any target antigen may be utilized.

An antibody or fragment thereof may be used which is not conjugated to a therapeutic agent—referred to as a “naked” antibody or fragment thereof. In alternative embodiments, antibodies or fragments may be conjugated to one or more therapeutic and/or diagnostic agents. A wide variety of such therapeutic and diagnostic agents are known in the art, as discussed in more detail below, and any such known therapeutic or diagnostic agent may be used.

Techniques for preparing monoclonal antibodies against virtually any target antigen are well known in the art. See, for example, Kohler and Milstein, Nature 256: 495 (1975), and Coligan et al. (eds.), CURRENT PROTOCOLS IN IMMUNOLOGY, VOL. 1, pages 2.5.1-2.6.7 (John Wiley & Sons 1991). Briefly, monoclonal antibodies can be obtained by injecting mice with a composition comprising an antigen, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

MAbs can be isolated and purified from hybridoma cultures by a variety of well-established techniques. Such isolation techniques include affinity chromatography with Protein-A SEPHAROSE®, size-exclusion chromatography, and ion-exchange chromatography. See, for example, Coligan at pages 2.7.1-2.7.12 and pages 2.9.1-2.9.3. Also, see Baines et al., “Purification of Immunoglobulin G (IgG),” in METHODS IN MOLECULAR BIOLOGY, VOL. 10, pages 79-104 (The Humana Press, Inc. 1992).

After the initial raising of antibodies to the immunogen, the antibodies can be sequenced and subsequently prepared by recombinant techniques. Humanization and chimerization of murine antibodies and antibody fragments are well known to those skilled in the art. The use of antibody components derived from humanized, chimeric or human antibodies obviates potential problems associated with the immunogenicity of murine constant regions.

Chimeric Antibodies

A chimeric antibody is a recombinant protein in which the variable regions of a human antibody have been replaced by the variable regions of, for example, a mouse antibody, including the complementarity-determining regions (CDRs) of the mouse antibody. Chimeric antibodies exhibit decreased immunogenicity and increased stability when administered to a subject. General techniques for cloning murine immunoglobulin variable domains are disclosed, for example, in Orlandi et al., Proc. Nat'l Acad. Sci. USA 86: 3833 (1989). Techniques for constructing chimeric antibodies are well known to those of skill in the art. As an example, Leung et al., Hybridoma 13:469 (1994), produced an LL2 chimera by combining DNA sequences encoding the V_{κ} and V_{H} domains of murine LL2, an anti-CD22 monoclonal antibody, with respective human κ and IgG₁ constant region domains.

Humanized Antibodies

Techniques for producing humanized MAbs are well known in the art (see, e.g., Jones et al., Nature 321: 522 (1986), Riechmann et al., Nature 332: 323 (1988), Verhoeyen et al., Science 239: 1534 (1988), Carter et al., Proc. Nat'l Acad. Sci. USA 89: 4285 (1992), Sandhu, Crit. Rev. Biotech. 12: 437 (1992), and Singer et al., J. Immun. 150: 2844 (1993)). A chimeric or murine monoclonal antibody may be humanized by transferring the mouse CDRs from the heavy and light variable chains of the mouse immunoglobulin into the corresponding variable domains of a human antibody. The mouse framework regions (FR) in the chimeric monoclonal antibody are also replaced with human FR sequences. As simply transferring mouse CDRs into human FRs often results in a reduction or even loss of antibody affinity, additional modification might be required in order to restore the original affinity of the murine antibody. This can be accomplished by the replacement of one or more human residues in the FR regions with their murine counterparts to obtain an antibody that possesses good binding affinity to its epitope. See, for example, Tempest et al., Biotechnology 9:266 (1991) and Verhoeyen et al., Science 239: 1534 (1988). Generally, those human FR amino acid residues that differ from their murine counterparts and are located close to or touching one or more CDR amino acid residues would be candidates for substitution.

Human Antibodies

Methods for producing fully human antibodies using either combinatorial approaches or transgenic animals transformed with human immunoglobulin loci are known in the art (e.g., Mancini et al., 2004, New Microbiol. 27:315-28; Conrad and Scheller, 2005, Comb. Chem. High Throughput Screen. 8:117-26; Brekke and Loset, 2003, Curr. Opin. Phamacol. 3:544-50). A fully human antibody also can be constructed by genetic or chromosomal transfection methods, as well as phage display technology, all of which are known in the art. See for example, McCafferty et al., Nature 348:552-553 (1990). Such fully human antibodies are expected to exhibit even fewer side effects than chimeric or humanized antibodies and to function in vivo as essentially endogenous human antibodies. In certain embodiments, the claimed methods and procedures may utilize human antibodies produced by such techniques.

In one alternative, the phage display technique may be used to generate human antibodies (e.g., Dantas-Barbosa et al., 2005, Genet. Mol. Res. 4:126-40). Human antibodies may be generated from normal humans or from humans that exhibit a particular disease state, such as cancer (Dantas-Barbosa et al., 2005). The advantage to constructing human antibodies from a diseased individual is that the circulating antibody repertoire may be biased towards antibodies against disease-associated antigens.

In one non-limiting example of this methodology, Dantas-Barbosa et al. (2005) constructed a phage display library of human Fab antibody fragments from osteosarcoma patients. Generally, total RNA was obtained from circulating blood lymphocytes (Id.). Recombinant Fab were cloned from the μ, γ and κ chain antibody repertoires and inserted into a phage display library (Id). RNAs were converted to cDNAs and used to make Fab cDNA libraries using specific primers against the heavy and light chain immunoglobulin sequences (Marks et al., 1991, J. Mol. Biol. 222:581-97). Library construction was performed according to Andris-Widhopf et al. (2000, In: Phage Display Laboratory Manual, Barbas et al. (eds), 1^{st} edition, Cold Spring Harbor Laboratory Press, Cold Spring Harbor, N.Y. pp. 9.1 to 9.22). The final Fab fragments were digested with restriction endonucleases and inserted into the bacteriophage genome to make the phage display library. Such libraries may be screened by standard phage display methods, as known in the art (see, e.g., Pasqualini and Ruoslahti, 1996, Nature 380:364-366; Pasqualini, 1999, The Quart. J. Nucl. Med. 43:159-162).

Phage display can be performed in a variety of formats, for their review, see e.g. Johnson and Chiswell, Current Opinion in Structural Biology 3:5564-571 (1993). Human antibodies may also be generated by in vitro activated B-cells. See U.S. Pat. Nos. 5,567,610 and 5,229,275, incorporated herein by reference in their entirety. The skilled artisan will realize that these techniques are exemplary and any known method for making and screening human antibodies or antibody fragments may be utilized.

In another alternative, transgenic animals that have been genetically engineered to produce human antibodies may be used to generate antibodies against essentially any immunogenic target, using standard immunization protocols. Methods for obtaining human antibodies from transgenic mice are disclosed by Green et al., Nature Genet. 7:13 (1994), Lonberg et al., Nature 368:856 (1994), and Taylor et al., Int. Immun. 6:579 (1994). A non-limiting example of such a system is the XENOMOUSE® (e.g., Green et al., 1999, J. Immunol. Methods 231:11-23) from Abgenix (Fremont, Calif.). In the XENOMOUSE® and similar animals, the mouse antibody genes have been inactivated and replaced by functional human antibody genes, while the remainder of the mouse immune system remains intact.

The XENOMOUSE® was transformed with germline-configured YACs (yeast artificial chromosomes) that contained portions of the human IgH and Igκ loci, including the majority of the variable region sequences, along accessory genes and regulatory sequences. The human variable region repertoire may be used to generate antibody producing B-cells, which may be processed into hybridomas by known techniques. A XENOMOUSE® immunized with a target antigen will produce human antibodies by the normal immune response, which may be harvested and/or produced by standard techniques discussed above. A variety of strains of XENOMOUSE® are available, each of which is capable of producing a different class of antibody. Transgenically produced human antibodies have been shown to have therapeutic potential, while retaining the pharmacokinetic properties of normal human antibodies (Green et al., 1999). The skilled artisan will realize that the claimed compositions and methods are not limited to use of the XENOMOUSE® system but may utilize any transgenic animal that has been genetically engineered to produce human antibodies.

### Antibody Fragments

Antibody fragments which recognize specific epitopes can be generated by known techniques. Antibody fragments are antigen binding portions of an antibody, such as F(ab′)₂, Fab′, F(ab)₂, Fab, Fv, sFv and the like. F(ab′)₂ fragments can be produced by pepsin digestion of the antibody molecule and Fab′ fragments can be generated by reducing disulfide bridges of the F(ab′)₂ fragments. Alternatively, Fab′ expression libraries can be constructed (Huse et al., 1989, Science, 246:1274-1281) to allow rapid and easy identification of monoclonal Fab′ fragments with the desired specificity. F(ab)₂ fragments may be generated by papain digestion of an antibody.

A single chain Fv molecule (scFv) comprises a VL domain and a VH domain. The VL and VH domains associate to form a target binding site. These two domains are further covalently linked by a peptide linker (L). Methods for making scFv molecules and designing suitable peptide linkers are described in U.S. Pat. No. 4,704,692, U.S. Pat. No. 4,946,778, R. Raag and M. Whitlow, “Single Chain Fvs.” FASEB Vol 9:73-80 (1995) and R. E. Bird and B. W. Walker, “Single Chain Antibody Variable Regions,” TIBTECH, Vol 9: 132-137 (1991).

An antibody fragment can be prepared by proteolytic hydrolysis of the full length antibody or by expression in E. coli or another host of the DNA coding for the fragment. An antibody fragment can be obtained by pepsin or papain digestion of full length antibodies by conventional methods. These methods are described, for example, by Goldenberg, U.S. Pat. Nos. 4,036,945 and 4,331,647 and references contained therein. Also, see Nisonoff et al., Arch Biochem. Biophys. 89: 230 (1960); Porter, Biochem. J. 73: 119 (1959), Edelman et al., in METHODS IN ENZYMOLOGY VOL. 1, page 422 (Academic Press 1967), and Coligan at pages 2.8.1-2.8.10 and 2.10.-2.10.4.

Known Antibodies

Antibodies of use may be commercially obtained from a wide variety of known sources. For example, a variety of antibody secreting hybridoma lines are available from the American Type Culture Collection (ATCC, Manassas, Va.). A large number of antibodies against various disease targets, including but not limited to tumor-associated antigens, have been deposited at the ATCC and/or have published variable region sequences and are available for use in the claimed methods and compositions. See, e.g., U.S. Pat. Nos. 7,312,318; 7,282,567; 7,151,164; 7,074,403; 7,060,802; 7,056,509; 7,049,060; 7,045,132; 7,041,803; 7,041,802; 7,041,293; 7,038,018; 7,037,498; 7,012,133; 7,001,598; 6,998,468; 6,994,976; 6,994,852; 6,989,241; 6,974,863; 6,965,018; 6,964,854; 6,962,981; 6,962,813; 6,956,107; 6,951,924; 6,949,244; 6,946,129; 6,943,020; 6,939,547; 6,921,645; 6,921,645; 6,921,533; 6,919,433; 6,919,078; 6,916,475; 6,905,681; 6,899,879; 6,893,625; 6,887,468; 6,887,466; 6,884,594; 6,881,405; 6,878,812; 6,875,580; 6,872,568; 6,867,006; 6,864,062; 6,861,511; 6,861,227; 6,861,226; 6,838,282; 6,835,549; 6,835,370; 6,824,780; 6,824,778; 6,812,206; 6,793,924; 6,783,758; 6,770,450; 6,767,711; 6,764,688; 6,764,681; 6,764,679; 6,743,898; 6,733,981; 6,730,307; 6,720,155; 6,716,966; 6,709,653; 6,693,176; 6,692,908; 6,689,607; 6,689,362; 6,689,355; 6,682,737; 6,682,736; 6,682,734; 6,673,344; 6,653,104; 6,652,852; 6,635,482; 6,630,144; 6,610,833; 6,610,294; 6,605,441; 6,605,279; 6,596,852; 6,592,868; 6,576,745; 6,572,856; 6,566,076; 6,562,618; 6,545,130; 6,544,749; 6,534,058; 6,528,625; 6,528,269; 6,521,227; 6,518,404; 6,511,665; 6,491,915; 6,488,930; 6,482,598; 6,482,408; 6,479,247; 6,468,531; 6,468,529; 6,465,173; 6,461,823; 6,458,356; 6,455,044; 6,455,040, 6,451,310; 6,444,206; 6,441,143; 6,432,404; 6,432,402; 6,419,928; 6,413,726; 6,406,694; 6,403,770; 6,403,091; 6,395,276; 6,395,274; 6,387,350; 6,383,759; 6,383,484; 6,376,654; 6,372,215; 6,359,126; 6,355,481; 6,355,444; 6,355,245; 6,355,244; 6,346,246; 6,344,198; 6,340,571; 6,340,459; 6,331,175; 6,306,393; 6,254,868; 6,187,287; 6,183,744; 6,129,914; 6,120,767; 6,096,289; 6,077,499; 5,922,302; 5,874,540; 5,814,440; 5,798,229; 5,789,554; 5,776,456; 5,736,119; 5,716,595; 5,677,136; 5,587,459; 5,443,953, 5,525,338. These are exemplary only and a wide variety of other antibodies and their hybridomas are known in the art. The skilled artisan will realize that antibody sequences or antibody-secreting hybridomas against almost any disease-associated antigen may be obtained by a simple search of the ATCC, NCBI and/or USPTO databases for antibodies against a selected disease-associated target of interest. The antigen binding domains of the cloned antibodies may be amplified, excised, ligated into an expression vector, transfected into an adapted host cell and used for protein production, using standard techniques well known in the art.

Known antibodies of use include, but are not limited to, J591 (anti-PSMA, U.S. Pat. No. 7,514,078), hPAM4 (anti-mucin, U.S. Pat. No. 7,282,567), hA20 (anti-CD20, U.S. Pat. No. 7,251,164), hA19 (anti-CD19, U.S. Pat. No. 7,109,304), hIMMU31 (anti-AFP, U.S. Pat. No. 7,300,655), hLL1 (anti-CD74, U.S. Pat. No. 7,312,318,), hLL2 (anti-CD22, U.S. Pat. No. 7,074,403), hMu-9 (anti-CSAp, U.S. Pat. No. 7,387,773), hL243 (anti-HLA-DR, U.S. Pat. No. 7,612,180), hMN-14 (anti-CEACAM5, U.S. Pat. No. 6,676,924), hMN-15 (anti-CEACAM6, U.S. Pat. No. 7,541,440), hR1 (anti-IGF-1R, U.S. Provisional Patent Application 61/145,896), hRS7 (anti-EGP-1, U.S. Pat. No. 7,238,785), hMN-3 (anti-CEACAM6, U.S. Pat. No. 7,541,440), AB-PG1-XG1-026 (anti-PSMA, U.S. patent application Ser. No. 11/983,372, deposited as ATCC PTA-4405 and PTA-4406), 29H2 (ABCAM®, Cambridge, Mass.) and D2/B (anti-PSMA, WO 2009/130575) the text of each recited patent or application is incorporated herein by reference with respect to the Figures and Examples sections. In certain embodiments, the antibody may be selected from any anti-hapten antibody known in the art, including but not limited to h679 (anti-HSG, U.S. Pat. No. 7,429,381) and 734 (anti-In-DTPA, U.S. Pat. No. 7,405,320), the text of each of which is incorporated herein by reference.

Other antibodies are known for therapy of diseases other than cancer or autoimmune disease. For example, bapineuzumab is in clinical trials for Alzheimer's disease therapy. Other antibodies proposed for therapy of Alzheimer's disease include Alz 50 (Ksiezak-Reding et al., 1987, J Biol Chem 263:7943-47), gantenerumab, and solanezumab. Infliximab, an anti-TNF-α antibody, has been reported to reduce amyloid plaques and improve cognition. Anti-CD3 antibodies have been proposed for therapy of type 1 diabetes (Cernea et al., 2010, Diabetes Metab Rev 26:602-05). Antibodies to fibrin (e.g., scFv (59D8); T2G1s; MH1) are known and in clinical trials as imaging agents for disclosing fibrin clots and pulmonary emboli, while anti-granulocyte antibodies, such as MN-3, MN-15, anti-NCA95, and anti-CD15 antibodies, can target myocardial infarcts and myocardial ischemia. (See, e.g., U.S. Pat. Nos. 5,487,892; 5,632,968; 6,294,173; 7,541,440, the Examples section of each incorporated herein by reference) Anti-macrophage, anti-low-density lipoprotein (LDL) and anti-CD74 (e.g., hLL1) antibodies can be used to target atherosclerotic plaques. Abciximab (anti-glycoprotein 10b/IIIa) has been approved for adjuvant use for prevention of restenosis in percutaneous coronary interventions and the treatment of unstable angina (Waldmann et al., 2000, Hematol 1:394-408). Anti-CD3 antibodies have been reported to reduce development and progression of atherosclerosis (Steffens et al., 2006, Circulation 114:1977-84). Antibodies against oxidized LDL induced a regression of established atherosclerosis in a mouse model (Ginsberg, 2007, J Am Coll Cardiol 52:2319-21). Anti-ICAM-1 antibody was shown to reduce ischemic cell damage after cerebral artery occlusion in rats (Zhang et al., 1994, Neurology 44:1747-51). Commercially available monoclonal antibodies to leukocyte antigens are represented by: OKT anti-T cell monoclonal antibodies (available from Ortho Pharmaceutical Company) which bind to normal T-lymphocytes; the monoclonal antibodies produced by the hybridomas having the ATCC accession numbers HB44, HB55, HB12, HB78 and HB2; G7Ell, W8E7, NKP15 and GO22 (Becton Dickinson); NEN9.4 (New England Nuclear); and FMCll (Sera Labs). A description of antibodies against fibrin and platelet antigens is contained in Knight, Semin. Nucl. Med., 20:52-67 (1990).

Other Disease-Associated Target Antigens

In one embodiment, a target may be an antigen or receptor of the adaptive immune system. In other embodiments, the target of the antibody complex may occur on cells of the innate immune system, such as granulocytes, monocytes, macrophages, dendritic cells, and NK-cells. Other targets include platelets and endothelial cells. Yet another group of targets is the group consisting of C5a, LPS, IFNγ and B7. A further group of suitable targets include CD2, CD3, CD4, CD14, CD18, CD11a, CD20, CD22, CD23, CD25, CD29, CD38, CD40L, CD52, CD64, CD83, CD147, and CD154. The CDs are targets on immune cells, which can be blocked to prevent an immune cell response. CD83 is particularly useful as a marker of activated dendritic cells (Cao et al., Biochem J., Aug. 23, 2004 (Epub ahead of print); Zinser et al., J. Exp Med. 200(3):345-51 (2004)).

Certain targets are of particular interest, such as MIF, HMGB-1, TNF-α, the complement factors and complement regulatory proteins, and the coagulation factors. MIF is a pivotal cytokine of the innate immune system and plays an important part in the control of inflammatory responses. MIF is released from macrophages and T lymphocytes that have been stimulated by glucocorticoids. Once released, MIF overcomes the inhibitory effects of glucocorticoids on TNF-α, IL-1 beta, IL-6, and IL-8 production by LPS-stimulated monocytes in vitro and suppresses the protective effects of steroids against lethal endotoxemia in vivo. MIF also antagonizes glucocorticoid inhibition of T-cell proliferation in vitro by restoring IL-2 and IFN-gamma production. MIF is the first mediator to be identified that can counter-regulate the inhibitory effects of glucocorticoids and thus plays a critical role in the host control of inflammation and immunity. MIF is particularly useful in treating cancer, pathological angiogenesis, and sepsis or septic shock.

HMGB-1, a DNA binding nuclear and cytosolic protein, is a proinflammatory cytokine released by monocytes and macrophages that have been activated by IL-1β, TNF, or LPS. Via its B box domain, it induces phenotypic maturation of DCs. It also causes increased secretion of the proinflammatory cytokines IL-1 alpha, IL-6, IL-8, IL-12, TNF-α and RANTES. HMGB-1 released by necrotic cells may be a signal of tissue or cellular injury that, when sensed by DCs, induces and/or enhances an immune reaction. Palumbo et al. report that HMBG1 induces mesoangioblast migration and proliferation (J Cell Biol, 164:441-449 (2004)).

HMGB-1 is a late mediator of endotoxin-induced lethality that exhibits significantly delayed kinetics relate to TNF and IL-1beta. Experimental therapeutics that target specific early inflammatory mediators such as TNF and IL-1beta alone have not proven efficacious in the clinic, but antibody complexes can improve response by targeting both early and late inflammatory mediators.

Antibody complexes that target HMBG-1 are especially useful in treating arthritis, particularly collagen-induced arthritis. Antibody complexes comprising HMBG-1 also are useful in treating sepsis and/or septic shock. Yang et al., PNAS USA 101:296-301 (2004); Kokkola et al., Arthritis Rheum, 48:2052-8 (2003); Czura et al., J Infect Dis, 187 Suppl 2:S391-6 (2003); Treutiger et al., J Intern Med, 254:375-85 (2003).

TNF-α is an important cytokine involved in systemic inflammation and the acute phase response. TNF-α is released by stimulated monocytes, fibroblasts, and endothelial cells. Macrophages, T-cells and B-lymphocytes, granulocytes, smooth muscle cells, eosinophils, chondrocytes, osteoblasts, mast cells, glial cells, and keratinocytes also produce TNF-α after stimulation. Its release is stimulated by several other mediators, such as interleukin-1 and bacterial endotoxin, in the course of damage, e.g., by infection. It has a number of actions on various organ systems, generally together with interleukins-1 and -6. One of the actions of TNF-α is appetite suppression; hence antibody complexes for treating cachexia preferably target TNF-α. It also stimulates the acute phase response of the liver, leading to an increase in C-reactive protein and a number of other mediators. It also is a useful target when treating sepsis or septic shock.

The complement system is a complex cascade involving proteolytic cleavage of serum glycoproteins often activated by cell receptors. The “complement cascade” is constitutive and non-specific but it must be activated in order to function. Complement activation results in a unidirectional sequence of enzymatic and biochemical reactions. In this cascade, a specific complement protein, C5, forms two highly active, inflammatory byproducts, C5a and C5b, which jointly activate white blood cells. This in turn evokes a number of other inflammatory byproducts, including injurious cytokines, inflammatory enzymes, and cell adhesion molecules. Together, these byproducts can lead to the destruction of tissue seen in many inflammatory diseases. This cascade ultimately results in induction of the inflammatory response, phagocyte chemotaxis and opsonization, and cell lysis.

The complement system can be activated via two distinct pathways, the classical pathway and the alternate pathway. Most of the complement components are numbered (e.g., C1, C2, C3, etc.) but some are referred to as “Factors.” Some of the components must be enzymatically cleaved to activate their function; others simply combine to form complexes that are active. Active components of the classical pathway include C1q, C1r, C1s, C2a, C2b, C3a, C3b, C4a, and C4b. Active components of the alternate pathway include C3a, C3b, Factor B, Factor Ba, Factor Bb, Factor D, and Properdin. The last stage of each pathway is the same, and involves component assembly into a membrane attack complex. Active components of the membrane attack complex include C5a, C5b, C6, C7, C8, and C9n.

While any of these components of the complement system can be targeted by an antibody complex, certain of the complement components are preferred. C3a, C4a and C5a cause mast cells to release chemotactic factors such as histamine and serotonin, which attract phagocytes, antibodies and complement, etc. These form one group of preferred targets. Another group of preferred targets includes C3b, C4b and C5b, which enhance phagocytosis of foreign cells. Another preferred group of targets are the predecessor components for these two groups, i.e., C3, C4 and C5. C5b, C6, C7, C8 and C9 induce lysis of foreign cells (membrane attack complex) and form yet another preferred group of targets.

Complement C5a, like C3a, is an anaphylatoxin. It mediates inflammation and is a chemotactic attractant for induction of neutrophilic release of antimicrobial proteases and oxygen radicals. Therefore, C5a and its predecessor C5 are particularly preferred targets. By targeting C5, not only is C5a affected, but also C5b, which initiates assembly of the membrane-attack complex. Thus, C5 is another preferred target. C3b, and its predecessor C3, also are preferred targets, as both the classical and alternate complement pathways depend upon C3b. Three proteins affect the levels of this factor, C1 inhibitor, protein H and Factor I, and these are also preferred targets according to the invention. Complement regulatory proteins, such as CD46, CD55, and CD59, may be targets to which the antibody complexes bind.

Coagulation factors also are preferred targets, particularly tissue factor and thrombin. Tissue factor is also known also as tissue thromboplastin, CD142, coagulation factor III, or factor III. Tissue factor is an integral membrane receptor glycoprotein and a member of the cytokine receptor superfamily. The ligand binding extracellular domain of tissue factor consists of two structural modules with features that are consistent with the classification of tissue factor as a member of type-2 cytokine receptors. Tissue factor is involved in the blood coagulation protease cascade and initiates both the extrinsic and intrinsic blood coagulation cascades by forming high affinity complexes between the extracellular domain of tissue factor and the circulating blood coagulation factors, serine proteases factor VII or factor VIIa. These enzymatically active complexes then activate factor IX and factor X, leading to thrombin generation and clot formation.

Tissue factor is expressed by various cell types, including monocytes, macrophages and vascular endothelial cells, and is induced by IL-1, TNF-α or bacterial lipopolysaccharides. Protein kinase C is involved in cytokine activation of endothelial cell tissue factor expression. Induction of tissue factor by endotoxin and cytokines is an important mechanism for initiation of disseminated intravascular coagulation seen in patients with Gram-negative sepsis. Tissue factor also appears to be involved in a variety of non-hemostatic functions including inflammation, cancer, brain function, immune response, and tumor-associated angiogenesis. Thus, antibody complexes that target tissue factor are useful not only in the treatment of coagulopathies, but also in the treatment of sepsis, cancer, pathologic angiogenesis, and other immune and inflammatory dysregulatory diseases according to the invention. A complex interaction between the coagulation pathway and the cytokine network is suggested by the ability of several cytokines to influence tissue factor expression in a variety of cells and by the effects of ligand binding to the receptor. Ligand binding (factor VIIa) has been reported to give an intracellular calcium signal, thus indicating that tissue factor is a true receptor.

Thrombin is the activated form of coagulation factor II (prothrombin); it converts fibrinogen to fibrin. Thrombin is a potent chemotaxin for macrophages, and can alter their production of cytokines and arachidonic acid metabolites. It is of particular importance in the coagulopathies that accompany sepsis. Numerous studies have documented the activation of the coagulation system either in septic patients or following LPS administration in animal models. Despite more than thirty years of research, the mechanisms of LPS-induced liver toxicity remain poorly understood. It is now clear that they involve a complex and sequential series of interactions between cellular and humoral mediators. In the same period of time, gram-negative systemic sepsis and its sequalae have become a major health concern, attempts to use monoclonal antibodies directed against LPS or various inflammatory mediators have yielded only therapeutic failures. antibody complexes that target both thrombin and at least one other target address the clinical failures in sepsis treatment.

In other embodiments, the antibody complexes bind to a MHC class I, MHC class II or accessory molecule, such as CD40, CD54, CD80 or CD86. The antibody complex also may bind to a T-cell activation cytokine, or to a cytokine mediator, such as NF-κB.

In certain embodiments, one of the two different targets may be a cancer cell receptor or cancer-associated antigen, particularly one that is selected from the group consisting of B-cell lineage antigens (CD19, CD20, CD21, CD22, CD23, etc.), VEGF, VEGFR, EGFR, carcinoembryonic antigen (CEA), placental growth factor (P1GF), tenascin, HER-2/neu, EGP-1, EGP-2, CD25, CD30, CD33, CD38, CD40, CD45, CD52, CD74, CD80, CD138, NCA66, CEACAM1, CEACAM6 (carcinoembryonic antigen-related cellular adhesion molecule 6), MUC1, MUC2, MUC3, MUC4, MUC16, IL-6, α-fetoprotein (AFP), A3, CA125, colon-specific antigen-p (CSAp), folate receptor, HLA-DR, human chorionic gonadotropin (HCG), Ia, EL-2, insulin-like growth factor (IGF) and IGF receptor, KS-1, Le(y), MAGE, necrosis antigens, PAM-4, prostatic acid phosphatase (PAP), Pr1, prostate specific antigen (PSA), prostate specific membrane antigen (PSMA), S100, T101, TAC, TAG72, TRAIL receptors, and carbonic anhydrase IX.

Targets associated with sepsis and immune dysregulation and other immune disorders include MIF, IL-1, IL-6, IL-8, CD74, CD83, and C5aR. Antibodies and inhibitors against C5aR have been found to improve survival in rodents with sepsis (Huber-Lang et al., FASEB J2002; 16:1567-1574; Riedemann et al., J Clin Invest 2002; 110:101-108) and septic shock and adult respiratory distress syndrome in monkeys (Hangen et al., J Surg Res 1989; 46:195-199; Stevens et al., J Clin Invest 1986; 77:1812-1816). Thus, for sepsis, one of the two different targets preferably is a target that is associated with infection, such as LPS/C5a. Other preferred targets include HMGB-1, tissue factor, CD14, VEGF, and IL-6, each of which is associated with septicemia or septic shock. Preferred antibody complexes are those that target two or more targets from HMGB-1, tissue factor and MIF, such as MIF/tissue factor, and HMGB-1/tissue factor.

In still other embodiments, one of the different targets may be a target that is associated with graft versus host disease or transplant rejection, such as MIF (Lo et al., Bone Marrow Transplant, 30(6):375-80 (2002)). One of the different targets also may be one that associated with acute respiratory distress syndrome, such as IL-8 (Bouros et al., PMC Pulm Med, 4(1):6 (2004), atherosclerosis or restenosis, such as MIF (Chen et al., Arterioscler Thromb Vasc Biol, 24(4):709-14 (2004), asthma, such as IL-18 (Hata et al., Int Immunol, Oct. 11, 2004 Epub ahead of print), a granulomatous disease, such as TNF-α (Ulbricht et al., Arthritis Rheum, 50(8):2717-8 (2004), a neuropathy, such as carbamylated EPO (erythropoietin) (Leist et al., Science 305(5681):164-5 (2004), or cachexia, such as IL-6 and TNF-α.

Other targets include C5a, LPS, IFN-gamma, B7; CD2, CD4, CD14, CD18, CD11a, CD11b, CD11c, CD14, CD18, CD27, CD29, CD38, CD40L, CD52, CD64, CD83, CD147, CD154. Activation of mononuclear cells by certain microbial antigens, including LPS, can be inhibited to some extent by antibodies to CD18, CD11b, or CD11c, which thus implicate β₂-integrins (Cuzzola et al., J Immunol 2000; 164:5871-5876; Medvedev et al., J Immunol 1998; 160: 4535-4542). CD83 has been found to play a role in giant cell arteritis (GCA), which is a systemic vasculitis that affects medium- and large-size arteries, predominately the extracranial branches of the aortic arch and of the aorta itself, resulting in vascular stenosis and subsequent tissue ischemia, and the severe complications of blindness, stroke and aortic arch syndrome (Weyand and Goronzy, N Engl J Med 2003; 349:160-169; Hunder and Valente, In: Inflammatory Diseases of Blood Vessels. G. S. Hoffman and C. M. Weyand, eds, Marcel Dekker, New York, 2002; 255-265). Antibodies to CD83 were found to abrogate vasculitis in a SCID mouse model of human GCA (Ma-Krupa et al., J Exp Med 2004; 199:173-183), suggesting to these investigators that dendritic cells, which express CD83 when activated, are critical antigen-processing cells in GCA. In these studies, they used a mouse anti-CD83 MAb (IgG1 clone HB15e from Research Diagnostics). CD154, a member of the TNF family, is expressed on the surface of CD4-positive T-lymphocytes, and it has been reported that a humanized monoclonal antibody to CD154 produced significant clinical benefit in patients with active systemic lupus erythematosus (SLE) (Grammar et al., J Clin Invest 2003; 112:1506-1520). It also suggests that this antibody might be useful in other autoimmune diseases (Kelsoe, J Clin Invest 2003; 112:1480-1482). Indeed, this antibody was also reported as effective in patients with refractory immune thrombocytopenic purpura (Kuwana et al., Blood 2004; 103:1229-1236).

In rheumatoid arthritis, a recombinant interleukin-1 receptor antagonist, IL-1Ra or anakinra, has shown activity (Cohen et al., Ann Rheum Dis 2004; 63:1062-8; Cohen, Rheum Dis Clin North Am 2004; 30:365-80). An improvement in treatment of these patients, which hitherto required concomitant treatment with methotrexate, is to combine anakinra with one or more of the anti-proinflammatory effector cytokines or anti-proinflammatory effector chemokines (as listed above). Indeed, in a review of antibody therapy for rheumatoid arthritis, Taylor (Curr Opin Pharmacol 2003; 3:323-328) suggests that in addition to TNF, other antibodies to such cytokines as IL-1, IL-6, IL-8, IL-15, IL-17 and IL-18, are useful.

Some of the more preferred target combinations are shown in Table 1. This is a list of examples of preferred combinations, but is not intended to be exhaustive.

TABLE 1
Potential Combinations of Target Antigens for Antibody Complexes

| First target | Second target |
|---|---|
| MIF | A second proinflammatory effector cytokine, especially HMGB-1, |
| | TNF-α, IL-1, or IL-6 |
| MIF | Proinflammatory effector chemokine, especially MCP-1, RANTES, MIP- |
| | 1A, or MIP-1B |
| MIF | Proinflammatory effector receptor, especially IL-6R IL-13R, and IL-15R |
| MIF | Coagulation factor, especially tissue factor or thrombin |
| MIF | Complement factor, especially C3, C5, C3a, or C5a |
| MIF | Complement regulatory protein, especially CD46, CD55, CD59, and |
| | mCRP |
| MIF | Cancer associated antigen or receptor |
| HMGB-1 | A second proinflammatory effector cytokine, especially MIF, TNF-α, IL-1, |
| | or IL-6 |
| HMGB-1 | Proinflammatory effector chemokine, especially MCP-1, RANTES, MIP- |
| | 1A, or MIP-1B |
| HMGB-1 | Proinflammatory effector receptor especially MCP-1, RANTES, MIP-1A, |
| | or MIP-1B |
| HMGB-1 | Coagulation factor, especially tissue factor or thrombin |
| HMGB-1 | Complement factor, especially C3, C5, C3a, or C5a |
| HMGB-1 | Complement regulatory protein, especially CD46, CD55, CD59, and |
| | mCRP |
| HMGB-1 | Cancer associated antigen or receptor |
| TNF-α | A second proinflammatory effector cytokine, especially MIF, HMGB-1, |
| | TNF-α, IL-1, or IL-6 |
| TNF-α | Proinflammatory effector chemokine, especially MCP-1, RANTES, MIP- |
| | 1A, or MIP-1B |
| TNF-α | Proinflammatory effector receptor, especially IL-6R IL-13R, and IL-15R |
| TNF-α | Coagulation factor, especially tissue factor or thrombin |
| TNF-α | Complement factor, especially C3, C5, C3a, or C5a |
| TNF-α | Complement regulatory protein, especially CD46, CD55, CD59, and |
| | mCRP |
| TNF-α | Cancer associated antigen or receptor |
| LPS | Proinflammatory effector cytokine, especially MIF, HMGB-1, |
| | TNF-α, IL-1, or IL-6 |
| LPS | Proinflammatory effector chemokine, especially MCP-1, RANTES, MIP- |
| | 1A, or MIP-1B |
| LPS | Proinflammatory effector receptor, especially IL-6R IL-13R, and IL-15R |
| LPS | Coagulation factor, especially tissue factor or thrombin |
| LPS | Complement factor, especially C3, C5, C3a, or C5a |
| LPS | Complement regulatory protein, especially CD46, CD55, CD59, and |
| | mCRP |
| Tissue factor | Proinflammatory effector cytokine, especially MIF, HMGB-1, |
| or thrombin | TNF-α, IL-1, or IL-6 |
| Tissue factor | Proinflammatory effector chemokine, especially MCP-1, RANTES, MIP- |
| or thrombin | 1A, or MIP-1B |
| Tissue factor | Proinflammatory effector receptor, especially IL-6R IL-13R, and IL-15R |
| or thrombin | |
| Tissue factor | Complement factor, especially C3, C5, C3a, or C5a |
| or thrombin | |
| Tissue factor | Complement regulatory protein, especially CD46, CD55, CD59, and |
| or thrombin | mCRP |
| Tissue factor | Cancer associated antigen or receptor |
| or thrombin | |

### Pre-Targeting

In certain embodiments, therapeutic agents may be administered by a pretargeting method, utilizing bispecific or multispecific antibody complexes. In pretargeting, the bispecific or multispecific antibody comprises at least one binding arm that binds to an antigen exhibited by a targeted cell or tissue, while at least one other binding arm binds to a hapten on a targetable construct. The targetable construct comprises one or more haptens and one or more therapeutic and/or diagnostic agents.

Pre-targeting is a multistep process originally developed to resolve the slow blood clearance of directly targeting antibodies, which contributes to undesirable toxicity to normal tissues such as bone marrow. With pre-targeting, a radionuclide or other diagnostic or therapeutic agent is attached to a small delivery molecule (targetable construct) that is cleared within minutes from the blood. A pre-targeting bispecific or multispecific antibody, which has binding sites for the targetable construct as well as a target antigen, is administered first, free antibody is allowed to clear from circulation and then the targetable construct is administered.

Pre-targeting methods are disclosed, for example, in Goodwin et al., U.S. Pat. No. 4,863,713; Goodwin et al., J. Nucl. Med. 29:226, 1988; Hnatowich et al., J. Nucl. Med. 28:1294, 1987; Oehr et al., J. Nucl. Med. 29:728, 1988; Klibanov et al., J. Nucl. Med. 29:1951, 1988; Sinitsyn et al., J. Nucl. Med. 30:66, 1989; Kalofonos et al., J. Nucl. Med. 31:1791, 1990; Schechter et al., Int. J. Cancer 48:167, 1991; Paganelli et al., Cancer Res. 51:5960, 1991; Paganelli et al., Nucl. Med. Commun. 12:211, 1991; U.S. Pat. No. 5,256,395; Stickney et al., Cancer Res. 51:6650, 1991; Yuan et al., Cancer Res. 51:3119, 1991; U.S. Pat. Nos. 6,077,499; 7,011,812; 7,300,644; 7,074,405; 6,962,702; 7,387,772; 7,052,872; 7,138,103; 6,090,381; 6,472,511; 6,962,702; and 6,962,702, each incorporated herein by reference.

A pre-targeting method of treating or diagnosing a disease or disorder in a subject may be provided by: (1) administering to the subject an antibody complex comprising a bispecific antibody or antibody fragment; (2) optionally administering to the subject a clearing composition, and allowing the composition to clear the antibody from circulation; and (3) administering to the subject the targetable construct, containing one or more chelated or chemically bound therapeutic or diagnostic agents.

Therapeutic Agents

A wide variety of therapeutic reagents can be administered concurrently or sequentially with the subject antibody complexes. For example, drugs, toxins, oligonucleotides, immunomodulators, hormones, hormone antagonists, enzymes, enzyme inhibitors, radionuclides, angiogenesis inhibitors, other antibodies or fragments thereof, etc. The therapeutic agents recited here are those agents that useful for administration separately with an antibody complex or else conjugated to a subject antibody complex. Therapeutic agents include, for example, chemotherapeutic drugs such as vinca alkaloids, anthracyclines, gemcitabine, epipodophyllotoxins, taxanes, antimetabolites, alkylating agents, antibiotics, SN-38, COX-2 inhibitors, antimitotics, anti-angiogenic and pro-apoptotic agents, particularly doxorubicin, methotrexate, taxol, CPT-11, camptothecans, proteosome inhibitors, mTOR inhibitors, HDAC inhibitors, tyrosine kinase inhibitors, and others.

Antisense molecules may include antisense molecules that correspond to bcl-2 or p53. However, other antisense molecules are known in the art, as described below, and any such known antisense molecule may be used. Second antibodies or fragments thereof may bind to an antigen selected from the group consisting of carbonic anhydrase IX, CCCL19, CCCL21, CSAp, CD1, CD1a, CD2, CD3, CD4, CD5, CD8, CD11A, CD14, CD15, CD16, CD18, CD19, IGF-1R, CD20, CD21, CD22, CD23, CD25, CD29, CD30, CD32b, CD33, CD37, CD38, CD40, CD40L, CD45, CD46, CD52, CD54, CD55, CD59, CD64, CD66a-e, CD67, CD70, CD74, CD79a, CD80, CD83, CD95, CD126, CD133, CD138, CD147, CD154, CXCR4, CXCR7, CXCL12, HIF-1a, AFP, PSMA, CEACAM1, CEACAM5, CEACAM6, B7, ED-B of fibronectin, Factor H, FHL-1, Flt-3, folate receptor, GROB, HMGB-1, hypoxia inducible factor (HIF), HM1.24, insulin-like growth factor-1 (IGF-1), IFN-γ, IFN-α, IFN-β, IL-2, IL-4R, IL-6R, IL-13R, IL-15R, IL-17R, IL-18R, IL-6, IL-8, IL-12, IL-15, IL-17, IL-18, IL-25, IP-10, MAGE, mCRP, MCP-1, MIP-1A, MIP-1B, MIF, MUC1, MUC2, MUC3, MUC4, MUC5, NCA-95, NCA-90, Ia, HM1.24, EGP-1, EGP-2, HLA-DR, tenascin, Le(y), RANTES, T101, TAC, Tn antigen, Thomson-Friedenreich antigens, tumor necrosis antigens, TNF-α, TRAIL receptor (R1 and R2), VEGF, EGFR, P1GF, complement factors C3, C3a, C3b, C5a, C5, and an oncogene product.

The therapeutic agent may be selected from the group consisting of aplidin, azaribine, anastrozole, azacytidine, bleomycin, bortezomib, bryostatin-1, busulfan, calicheamycin, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, PSI-341, semustine streptozocin, tamoxifen, taxanes, taxol, testosterone propionate, thalidomide, thioguanine, thiotepa, teniposide, topotecan, uracil mustard, velcade, vinblastine, vinorelbine, vincristine, ricin, abrin, ribonuclease, onconase, rapLRI, DNase I, Staphylococcal enterotoxin-A, pokeweed antiviral protein, gelonin, diphtheria toxin, Pseudomonas exotoxin, and Pseudomonas endotoxin.

Particularly useful therapeutic radionuclides include, but are not limited to ¹¹¹In, ¹⁷⁷Lu, ²¹²Bi, ²¹³Bi, ²¹¹At, ⁶²Cu, ⁶⁴Cu, ⁶⁷Cu, ⁹⁰Y, ¹²⁵I, ¹³¹I, ³²P, ³³P, ⁴⁷Sc, ¹¹¹Ag, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, and ²¹¹Pb. The therapeutic radionuclide preferably has a decay energy in the range of 20 to 6,000 keV, preferably in the ranges 60 to 200 keV for an Auger emitter, 100-2,500 keV for a beta emitter, and 4,000-6,000 keV for an alpha emitter. Maximum decay energies of useful beta-particle-emitting nuclides are preferably 20-5,000 keV, more preferably 100-4,000 keV, and most preferably 500-2,500 keV. Also preferred are radionuclides that substantially decay with Auger-emitting particles. For example, Co-58, Ga-67, Br-80m, Tc-99m, Rh-103m, Pt-109, In-111, Sb-119, I-125, Ho-161, Os-189m and Ir-192. Decay energies of useful beta-particle-emitting nuclides are preferably _{<}1,000 keV, more preferably <100 keV, and most preferably <70 keV. Also preferred are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213 and Fm-255. Decay energies of useful alpha-particle-emitting radionuclides are preferably 2,000-10,000 keV, more preferably 3,000-8,000 keV, and most preferably 4,000-7,000 keV.

Additional potential therapeutic radioisotopes include ¹¹C, ¹³N, ¹⁵O, ⁷⁵Br, ¹⁹⁸Au, ²²⁴Ac, ¹²⁶I, ¹³³I, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ^{125m}Te, ¹⁶⁵Tm, ¹⁶⁷Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵⁸Co, ⁵¹Cr, ⁵⁹Fe, ⁷⁵Se, ²⁰¹Tl, ²²⁵Ac, ⁷⁶Br, ¹⁶⁹Yb, and the like.

The therapeutic agent may be an enzyme selected from the group consisting of malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

An immunomodulator of use may be selected from the group consisting of a cytokine, a lymphokine, a monokine, a stem cell growth factor, a lymphotoxin, a hematopoietic factor, a colony stimulating factor (CSF), an interferon (IFN), parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, a transforming growth factor (TGF), TGF-α, TGF-β, insulin-like growth factor (IGF), erythropoietin, thrombopoietin, tumor necrosis factor (TNF), TNF-α, TNF-β, a mullerian-inhibiting substance, mouse gonadotropin-associated peptide, inhibin, activin, vascular endothelial growth factor, integrin, interleukin (IL), granulocyte-colony stimulating factor (G-CSF), granulocyte macrophage-colony stimulating factor (GM-CSF), interferon-α, interferon-β, interferon-γ, interferon-λ, S1 factor, IL-1, IL-1cc, IL-2, IL-3, IL-4, IL-5, IL-6, IL-7, IL-8, IL-9, IL-10, IL-11, IL-12, IL-13, IL-14, IL-15, IL-16, IL-17, IL-18 IL-21 and IL-25, LIF, kit-ligand, FLT-3, angiostatin, thrombospondin, endostatin and LT, and the like.

Exemplary anti-angiogenic agents may include angiostatin, endostatin, vasculostatin, canstatin, maspin, anti-VEGF binding molecules, anti-placental growth factor binding molecules, or anti-vascular growth factor binding molecules.

In certain embodiments, the antibody complex may comprise one or more chelating moieties, such as NOTA, DOTA, DTPA, TETA, Tscg-Cys, or Tsca-Cys. In certain embodiments, the chelating moiety may form a complex with a therapeutic or diagnostic cation, such as Group II, Group III, Group IV, Group V, transition, lanthanide or actinide metal cations, Tc, Re, Bi, Cu, As, Ag, Au, At, or Pb.

Other useful cancer chemotherapeutic drugs include nitrogen mustards, alkyl sulfonates, nitrosoureas, triazenes, folic acid analogs, COX-2 inhibitors, antimetabolites, pyrimidine analogs, purine analogs, platinum coordination complexes, mTOR inhibitors, tyrosine kinase inhibitors, proteosome inhibitors, HDAC inhibitors, camptothecins, hormones, and the like. Suitable chemotherapeutic agents are described in REMINGTON'S PHARMACEUTICAL SCIENCES, 19^{th} Ed. (Mack Publishing Co. 1995), and in GOODMAN AND GILMAN'S THE PHARMACOLOGICAL BASIS OF THERAPEUTICS, 7^{th} Ed. (MacMillan Publishing Co. 1985), as well as revised editions of these publications. Other suitable chemotherapeutic agents, such as experimental drugs, are known to those of skill in the art.

A toxin can be of animal, plant or microbial origin. A toxin, such as Pseudomonas exotoxin, may also be complexed to or form the therapeutic agent portion of an immunoconjugate. Other toxins include ricin, abrin, ribonuclease (RNase), DNase I, Staphylococcal enterotoxin-A, pokeweed antiviral protein, onconase, gelonin, diphtheria toxin, Pseudomonas exotoxin, and Pseudomonas endotoxin. See, for example, Pastan et al., Cell 47:641 (1986), Goldenberg, C A—A Cancer Journal for Clinicians 44:43 (1994), Sharkey and Goldenberg, C A—A Cancer Journal for Clinicians 56:226 (2006). Additional toxins suitable for use are known to those of skill in the art and are disclosed in U.S. Pat. No. 6,077,499, the Examples section of which is incorporated herein by reference.

Interference RNA

In certain preferred embodiments the therapeutic agent may be a siRNA or interference RNA species. The siRNA, interference RNA or therapeutic gene may be attached to a carrier moiety that is conjugated to an antibody or fragment in an antibody complex. A variety of carrier moieties for siRNA have been reported and any such known carrier may be incorporated into an antibody construct for use. Non-limiting examples of carriers include protamine (Rossi, 2005, Nat Biotech 23:682-84; Song et al., 2005, Nat Biotech 23:709-17); dendrimers such as PAMAM dendrimers (Pan et al., 2007, Cancer Res. 67:8156-8163); polyethylenimine (Schiffelers et al., 2004, Nucl Acids Res 32:e149); polypropyleneimine (Taratula et al., 2009, J Control Release 140:284-93); polylysine (Inoue et al., 2008, J Control Release 126:59-66); histidine-containing reducible polycations (Stevenson et al., 2008, J Control Release 130:46-56); histone H1 protein (Haberland et al., 2009, Mol Biol Rep 26:1083-93); cationic comb-type copolymers (Sato et al., 2007, J Control Release 122:209-16); polymeric micelles (U.S. Patent Application Publ. No. 20100121043); and chitosan-thiamine pyrophosphate (Rojanarata et al., 2008, Pharm Res 25:2807-14). The skilled artisan will realize that in general, polycationic proteins or polymers are of use as siRNA carriers. The skilled artisan will further realize that siRNA carriers can also be used to carry other oligonucleotide or nucleic acid species, such as anti-sense oligonucleotides or short DNA genes.

Known siRNA species of potential use include those specific for IKK-gamma (U.S. Pat. No. 7,022,828); VEGF, Flt-1 and Flk-1/KDR (U.S. Pat. No. 7,148,342); Bcl2 and EGFR (U.S. Pat. No. 7,541,453); CDC20 (U.S. Pat. No. 7,550,572); transducin (beta)-like 3 (U.S. Pat. No. 7,576,196); KRAS (U.S. Pat. No. 7,576,197); carbonic anhydrase II (U.S. Pat. No. 7,579,457); complement component 3 (U.S. Pat. No. 7,582,746); interleukin-1 receptor-associated kinase 4 (IRAK4) (U.S. Pat. No. 7,592,443); survivin (U.S. Pat. No. 7,608,7070); superoxide dismutase 1 (U.S. Pat. No. 7,632,938); MET proto-oncogene (U.S. Pat. No. 7,632,939); amyloid beta precursor protein (APP) (U.S. Pat. No. 7,635,771); IGF-1R (U.S. Pat. No. 7,638,621); ICAM1 (U.S. Pat. No. 7,642,349); complement factor B (U.S. Pat. No. 7,696,344); p53 (7,781,575), and apolipoprotein B (7,795,421), the Examples section of each referenced patent incorporated herein by reference.

Additional siRNA species are available from known commercial sources, such as Sigma-Aldrich (St Louis, Mo.), Invitrogen (Carlsbad, Calif.), Santa Cruz Biotechnology (Santa Cruz, Calif.), Ambion (Austin, Tex.), Dharmacon (Thermo Scientific, Lafayette, Colo.), Promega (Madison, Wis.), Mirus Bio (Madison, Wis.) and Qiagen (Valencia, Calif.), among many others. Other publicly available sources of siRNA species include the siRNAdb database at the Stockholm Bioinformatics Centre, the MIT/ICBP siRNA Database, the RNAi Consortium shRNA Library at the Broad Institute, and the Probe database at NCBI. For example, there are 30,852 siRNA species in the NCBI Probe database. The skilled artisan will realize that for any gene of interest, either a siRNA species has already been designed, or one may readily be designed using publicly available software tools. Any such siRNA species may be delivered using the subject antibody complexes.

Exemplary siRNA species known in the art are listed in Table 2. Although siRNA is delivered as a double-stranded molecule, for simplicity only the sense strand sequences are shown in Table 2.

TABLE 2
Exemplary siRNA Sequences

| Target | Sequence | SEQ ID NO |
|---|---|---|
| VEGF R2 | AATGCGGCGGTGGTGACAGTA | SEQ ID NO: 85 |
| VEGF R2 | AAGCTCAGCACACAGAAAGAC | SEQ ID NO: 86 |
| CXCR4 | UAAAAUCUUCCUGCCCACCdTdT | SEQ ID NO: 87 |
| CXCR4 | GGAAGCUGUUGGCUGAAAAdTdT | SEQ ID NO: 88 |
| PPARC1 | AAGACCAGCCUCUUUGCCCAG | SEQ ID NO: 89 |
| Dynamin 2 | GGACCAGGCAGAAAACGAG | SEQ ID NO: 90 |
| Catenin | CUAUCAGGAUGACGCGG | SEQ ID NO: 91 |
| E1A binding protein | UGACACAGGCAGGCUUGACUU | SEQ ID NO: 92 |
| Plasminogen | GGTGAAGAAGGGCGTCCAA | SEQ ID NO: 93 |
| activator | | |
| K-ras | GATCCGTTGGAGCTGTTGGCGTAGTT | SEQ ID NO: 94 |
| | CAAGAGACTCGCCAACAGCTCCAACT | |
| | TTTGGAAA | |
| Sortilin 1 | AGGTGGTGTTAACAGCAGAG | SEQ ID NO: 95 |
| Apolipoprotein E | AAGGTGGAGCAAGCGGTGGAG | SEQ ID NO: 96 |
| Apolipoprotein E | AAGGAGTTGAAGGCCGACAAA | SEQ ID NO: 97 |
| Bcl-X | UAUGGAGCUGCAGAGGAUGdTdT | SEQ ID NO: 98 |
| Raf-1 | TTTGAATATCTGTGCTGAGAACACA | SEQ ID NO: 99 |
| | GTTCTCAGCACAGATATTCTTTTT | |
| Heat shock | AATGAGAAAAGCAAAAGGTGCCCTGTCTC | SEQ ID NO: 100 |
| transcription | | |
| factor 2 | | |
| IGFBP3 | AAUCAUCAUCAAGAAAGGGCA | SEQ ID NO: 101 |
| Thioredoxin | AUGACUGUCAGGAUGUUGCdTdT | SEQ ID NO: 102 |
| CD44 | GAACGAAUCCUGAAGACAUCU | SEQ ID NO: 103 |
| MMP14 | AAGCCTGGCTACAGCAATATGCCTGTCTC | SEQ ID NO: 104 |
| MAPKAPK2 | UGACCAUCACCGAGUUUAUdTdT | SEQ ID NO: 105 |
| FGFR1 | AAGTCGGACGCAACAGAGAAA | SEQ ID NO: 106 |
| ERBB2 | CUACCUUUCUACGGACGUGdTdT | SEQ ID NO: 107 |
| BCL2L1 | CTGCCTAAGGCGGATTTGAAT | SEQ ID NO: 108 |
| ABL1 | TTAUUCCUUCUUCGGGAAGUC | SEQ ID NO: 109 |
| CEACAM1 | AACCTTCTGGAACCCGCCCAC | SEQ ID NO: 110 |
| CD9 | GAGCATCTTCGAGCAAGAA | SEQ ID NO: 111 |
| CD151 | CATGTGGCACCGTTTGCCT | SEQ ID NO: 112 |
| Caspase 8 | AACTACCAGAAAGGTATACCT | SEQ ID NO: 113 |
| BRCA1 | UCACAGUGUCCUUUAUGUAdTdT | SEQ ID NO: 114 |
| p53 | GCAUGAACCGGAGGCCCAUTT | SEQ ID NO: 115 |
| CEACAM6 | CCGGACAGTTCCATGTATA | SEQ ID NO: 116 |

The skilled artisan will realize that Table 2 represents a very small sampling of the total number of siRNA species known in the art, and that any such known siRNA may be utilized in the claimed methods and compositions.

Immunotoxins Comprising Ranpirnase (Rap)

Ribonucleases, in particular, Rap (Lee, Exp Opin Biol Ther 2008; 8:813-27) and its more basic variant, amphinase (Ardelt et al., Curr Pharm Biotechnol 2008:9:215-25), are potential anti-tumor agents (Lee and Raines, Biodrugs 2008; 22:53-8). Rap is a single-chain ribonuclease of 104 amino acids originally isolated from the oocytes of Rana pipiens. Rap exhibits cytostatic and cytotoxic effects on a variety of tumor cell lines in vitro, as well as antitumor activity in vivo. The amphibian ribonuclease enters cells via receptor-mediated endocytosis and once internalized into the cytosol, selectively degrades tRNA, resulting in inhibition of protein synthesis and induction of apoptosis.

Rap has completed a randomized Phase Mb clinical trial, which compared the effectiveness of Rap plus doxorubicin with that of doxorubicin alone in patients with unresectable malignant mesothelioma, with the interim analysis showing that the MST for the combination was 12 months, while that of the monotherapy was 10 months (Mutti and Gaudino, Oncol Rev 2008; 2:61-5). Rap can be administered repeatedly to patients without an untoward immune response, with reversible renal toxicity reported to be dose-limiting (Mikulski et al., J Clin Oncol 2002; 20:274-81; Int J Oncol 1993; 3:57-64).

Conjugation or fusion of Rap to a tumor-targeting antibody or antibody fragment is a promising approach to enhance its potency, as first demonstrated for LL2-onconase (Newton et al., Blood 2001; 97:528-35), a chemical conjugate comprising Rap and a murine anti-CD22 monoclonal antibody (MAb), and subsequently for 2L-Rap-hLL1-γ4P, a fusion protein comprising Rap and a humanized anti-CD74 MAb (Stein et al., Blood 2004; 104:3705-11).

The method used to generate 2L-Rap-hLL1-γ4P allowed us to develop a series of structurally similar immunotoxins, referred to in general as 2L-Rap-X, all of which consist of two Rap molecules, each connected via a flexible linker to the N-terminus of one L chain of an antibody of interest (X). We have also generated another series of immunotoxins of the same design, referred to as 2LRap(Q)-X, by substituting Rap with its non-glycosylation form of Rap, designated as Rap(Q) to denote that the potential glycosylation site at Asn69 is changed to Gln (or Q, single letter code). For both series, we made the IgG as either IgG1(γ1) or IgG4(γ4), and to prevent the formation of IgG4 half molecules (Aalberse and Schuurman, Immunology 2002; 105:9-19), we converted the serine residue in the hinge region (S228) of IgG4 to proline (γ4P). A pyroglutamate residue at the N-terminus of Rap is required for the RNase to be fully functional (Liao et al., Nucleic Acids Res 2003; 31:5247-55).

The skilled artisan will recognize that the cytotoxic RNase moieties suitable for use in the present invention include polypeptides having a native ranpirnase structure and all enzymatically active variants thereof. These molecules advantageously have an N-terminal pyroglutamic acid resides that appears essential for RNase activity and are not substantially inhibited by mammalian RNase inhibitors. Nucleic acid that encodes a native cytotoxic RNase may be prepared by cloning and restriction of appropriate sequences, or using DNA amplification with polymerase chain reaction (PCR). The amino acid sequence of Rana Pipiens ranpirnase can be obtained from Ardelt et al., J. Biol. Chem., 256: 245 (1991), and cDNA sequences encoding native ranpirnase, or a conservatively modified variation thereof, can be gene-synthesized by methods similar to the en bloc V-gene assembly method used in hLL2 humanization. (Leung et al., Mol. Immunol., 32: 1413, 1995). Methods of making cytotoxic RNase variants are known in the art and are within the skill of the routineer.

Rap conjugates of targeting antibodies may be made by standard techniques. The Rap-antibody constructs show potent cytotoxic activity that can be targeted to disease-associated cells.

Diagnostic Agents

An antibody complex may be administered conjugated to one or more diagnostic agents. Diagnostic agents are preferably selected from the group consisting of a radionuclide, a radiological contrast agent, a paramagnetic ion, a metal, a fluorescent label, a chemiluminescent label, an ultrasound contrast agent and a photoactive agent. Such diagnostic agents are well known and any such known diagnostic agent may be used. Non-limiting examples of diagnostic agents may include a radionuclide such as ¹¹⁰In, ¹¹¹In. ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ^{52m}Mn, ⁵⁵Co, ⁷²As, ⁷⁵Br, ⁷⁶Br, ^{82m}Rb, ⁸³Sr or other gamma-, beta-, or positron-emitters. Paramagnetic ions of use may include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III). Metal contrast agents may include lanthanum (III), gold (III), lead (II) or bismuth (III). Ultrasound contrast agents may comprise liposomes, such as gas filled liposomes. Radiopaque diagnostic agents may be selected from compounds, barium compounds, gallium compounds, and thallium compounds. A wide variety of fluorescent labels are known in the art, including but not limited to fluorescein isothiocyanate, rhodamine, phycoerytherin, phycocyanin, allophycocyanin, o-phthaldehyde and fluorescamine. Chemiluminescent labels of use may include luminol, isoluminol, an aromatic acridinium ester, an imidazole, an acridinium salt or an oxalate ester.

Conjugation

In preferred embodiments, an antibody or antibody fragment in a multivalent, multispecific antibody complex may be directly attached to one or more therapeutic agents to form an immunoconjugate. Therapeutic agents may be attached, for example to reduced SH groups and/or to carbohydrate side chains. A therapeutic agent can be attached at the hinge region of a reduced antibody component via disulfide bond formation. Alternatively, such agents can be attached using a heterobifunctional cross-linker, such as N-succinyl 3-(2-pyridyldithio)propionate (SPDP). Yu et al., Int. J. Cancer 56: 244 (1994). General techniques for such conjugation are well-known in the art. See, for example, Wong, CHEMISTRY OF PROTEIN CONJUGATION AND CROSS-LINKING (CRC Press 1991); Upeslacis et al., “Modification of Antibodies by Chemical Methods,” in MONOCLONAL ANTIBODIES: PRINCIPLES AND APPLICATIONS, Birch et al. (eds.), pages 187-230 (Wiley-Liss, Inc. 1995); Price, “Production and Characterization of Synthetic Peptide-Derived Antibodies,” in MONOCLONAL ANTIBODIES: PRODUCTION, ENGINEERING AND CLINICAL APPLICATION, Ritter et al. (eds.), pages 60-84 (Cambridge University Press 1995). Alternatively, the therapeutic agent can be conjugated via a carbohydrate moiety in the Fc region of the antibody.

Methods for conjugating functional groups to antibodies via an antibody carbohydrate moiety are well-known to those of skill in the art. See, for example, Shih et al., Int. J. Cancer 41: 832 (1988); Shih et al., Int. J. Cancer 46: 1101 (1990); and Shih et al., U.S. Pat. No. 5,057,313, the Examples section of which is incorporated herein by reference. The general method involves reacting an antibody having an oxidized carbohydrate portion with a carrier polymer that has at least one free amine function. This reaction results in an initial Schiff base (imine) linkage, which can be stabilized by reduction to a secondary amine to form the final conjugate.

The Fc region may be absent if the antibody component of the immunoconjugate is an antibody fragment. However, it is possible to introduce a carbohydrate moiety into the light chain variable region of a full length antibody or antibody fragment. See, for example, Leung et al., J. Immunol. 154: 5919 (1995); U.S. Pat. Nos. 5,443,953 and 6,254,868, the Examples section of which is incorporated herein by reference. The engineered carbohydrate moiety is used to attach the therapeutic or diagnostic agent.

An alternative method for attaching therapeutic agents to an antibody or other effector moiety involves use of click chemistry reactions. The click chemistry approach was originally conceived as a method to rapidly generate complex substances by joining small subunits together in a modular fashion. (See, e.g., Kolb et al., 2004, Angew Chem Int Ed 40:3004-31; Evans, 2007, Aust J Chem 60:384-95.) Various forms of click chemistry reaction are known in the art, such as the Huisgen 1,3-dipolar cycloaddition copper catalyzed reaction (Tornoe et al., 2002, J Organic Chem 67:3057-64), which is often referred to as the “click reaction.” Other alternatives include cycloaddition reactions such as the Diels-Alder, nucleophilic substitution reactions (especially to small strained rings like epoxy and aziridine compounds), carbonyl chemistry formation of urea compounds and reactions involving carbon-carbon double bonds, such as alkynes in thiol-yne reactions.

The azide alkyne Huisgen cycloaddition reaction uses a copper catalyst in the presence of a reducing agent to catalyze the reaction of a terminal alkyne group attached to a first molecule. In the presence of a second molecule comprising an azide moiety, the azide reacts with the activated alkyne to form a 1,4-disubstituted 1,2,3-triazole. The copper catalyzed reaction occurs at room temperature and is sufficiently specific that purification of the reaction product is often not required. (Rostovstev et al., 2002, Angew Chem Int Ed 41:2596; Tornoe et al., 2002, J Org Chem 67:3057.) The azide and alkyne functional groups are largely inert towards biomolecules in aqueous medium, allowing the reaction to occur in complex solutions. The triazole formed is chemically stable and is not subject to enzymatic cleavage, making the click chemistry product highly stable in biological systems. Although the copper catalyst is toxic to living cells, the copper-based click chemistry reaction may be used in vitro for immunoconjugate formation.

A copper-free click reaction has been proposed for covalent modification of biomolecules. (See, e.g., Agard et al., 2004, J Am Chem Soc 126:15046-47.) The copper-free reaction uses ring strain in place of the copper catalyst to promote a [3+2] azide-alkyne cycloaddition reaction (Id.) For example, cyclooctyne is an 8-carbon ring structure comprising an internal alkyne bond. The closed ring structure induces a substantial bond angle deformation of the acetylene, which is highly reactive with azide groups to form a triazole. Thus, cyclooctyne derivatives may be used for copper-free click reactions (Id.)

Another type of copper-free click reaction was reported by Ning et al. (2010, Angew Chem Int Ed 49:3065-68), involving strain-promoted alkyne-nitrone cycloaddition. To address the slow rate of the original cyclooctyne reaction, electron-withdrawing groups are attached adjacent to the triple bond (Id.) Examples of such substituted cyclooctynes include difluorinated cyclooctynes, 4-dibenzocyclooctynol and azacyclooctyne (Id.) An alternative copper-free reaction involved strain-promoted alkyne-nitrone cycloaddition to give N-alkylated isoxazolines (Id.) The reaction was reported to have exceptionally fast reaction kinetics and was used in a one-pot three-step protocol for site-specific modification of peptides and proteins (Id.) Nitrones were prepared by the condensation of appropriate aldehydes with N-methylhydroxylamine and the cycloaddition reaction took place in a mixture of acetonitrile and water (Id.) These and other known click chemistry reactions may be used to attach therapeutic agents to antibodies in vitro.

The specificity of the click chemistry reaction may be used as a substitute for the antibody-hapten binding interaction used in pretargeting with bispecific antibodies. In this alternative embodiment, the specific reactivity of e.g., cyclooctyne moieties for azide moieties or alkyne moieties for nitrone moieties may be used in an in vivo cycloaddition reaction. An antibody-based DNL complex is activated by incorporation of a substituted cyclooctyne, an azide or a nitrone moiety. A targetable construct is labeled with one or more diagnostic or therapeutic agents and a complementary reactive moiety. I.e., where the antibody comprises a cyclooctyne, the targetable construct will comprise an azide; where the antibody comprises a nitrone, the targetable construct will comprise an alkyne, etc. The DNL complex comprising an activated antibody is administered to a subject and allowed to localize to a targeted cell, tissue or pathogen, as disclosed for pretargeting protocols. The reactive labeled targetable construct is then administered. Because the cyclooctyne, nitrone or azide on the targetable construct is unreactive with endogenous biomolecules and highly reactive with the complementary moiety on the antibody, the specificity of the binding interaction results in the highly specific binding of the targetable construct to the tissue-localized antibody. Although the discussion above concerns click chemistry reactions with antibody effector moiety, the skilled artisan will realize that such reactions may be used to attach any functional groups to any effector moiety that may be incorporated into a DNL construct.

Therapeutic Use

The compositions described herein are particularly useful for treatment of various disease states. In preferred embodiments, the diseases may be autoimmune diseases or cancer, such as hematopoietic cancers or solid tumors. Exemplary non-limiting diseases that may be treated using the disclosed compositions and methods include indolent forms of B-cell lymphomas, aggressive forms of B-cell lymphomas, non-Hodgkin's lymphoma, multiple myeloma, chronic lymphatic leukemias, acute lymphatic leukemias, acute myelogenous leukemia, chronic lymphocytic leukemia, chronic myelogenous leukemia, Hodgkin's lymphoma, Waldenstrom's macroglobulinemia, GVHD, cryoglobulinemia, hemolytic anemia, allosensitization, septicemia, asthma and organ transplant rejection. Also included are class III autoimmune diseases such as immune-mediated thrombocytopenias, such as acute idiopathic thrombocytopenic purpura and chronic idiopathic thrombocytopenic purpura, dermatomyositis, Sjögren's syndrome, multiple sclerosis, Sydenham's chorea, myasthenia gravis, systemic lupus erythematosus, lupus nephritis, rheumatic fever, rheumatoid arthritis, polyglandular syndromes, bullous pemphigoid, diabetes mellitus, Henoch-Schönlein purpura, post-streptococcal nephritis, erythema nodosum, Takayasu's arteritis, Addison's disease, sarcoidosis, ulcerative colitis, erythema multiforme, IgA nephropathy, polyarteritis nodosa, ankylosing spondylitis, Goodpasture's syndrome, thromboangitis obliterans, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis and fibrosing alveolitis.

The antibody therapy can be further supplemented with the administration, either concurrently or sequentially, of at least one therapeutic agent, as discussed above. Multimodal therapies may include therapy supplemented with administration of anti-CD22, anti-CD19, anti-CD20, anti-CD21, anti-CD74, anti-CD80, anti-CD23, anti-CD45, anti-CD46, anti-MIF, anti-EGP-1, anti-CEACAM5, anti-CEACAM6, PAM4, or anti-HLA-DR (including the invariant chain) antibodies in the form of naked antibodies, fusion proteins, or as immunoconjugates. Various antibodies of use, such as anti-CD19, anti-CD20, and anti-CD22 antibodies, are known to those of skill in the art. See, for example, Ghetie et al., Cancer Res. 48:2610 (1988); Hekman et al., Cancer Immunol. Immunother. 32:364 (1991); Longo, Curr. Opin. Oncol. 8:353 (1996), U.S. Pat. Nos. 5,798,554; 6,187,287; 6,306,393; 6,676,924; 7,109,304; 7,151,164; 7,230,084; 7,230,085; 7,238,785; 7,238,786; 7,282,567; 7,300,655; 7,312,318; 7,612,180; 7,501,498 and U.S. Patent Application Publ. Nos. 20080131363; 20080089838; 20070172920; 20060193865; and 20080138333; the Examples section of each of which is incorporated herein by reference.

In another form of multimodal therapy, subjects receive naked antibodies, and/or immunoconjugates, in conjunction with standard cancer chemotherapy. For example, “CVB” (1.5 g/m² cyclophosphamide, 200-400 mg/m² etoposide, and 150-200 mg/m² carmustine) is a regimen used to treat non-Hodgkin's lymphoma. Patti et al., Eur. J. Haematol. 51: 18 (1993). Other suitable combination chemotherapeutic regimens are well-known to those of skill in the art. See, for example, Freedman et al., “Non-Hodgkin's Lymphomas,” in CANCER MEDICINE, VOLUME 2, 3^{rd} Edition, Holland et al. (eds.), pages 2028-2068 (Lea & Febiger 1993). As an illustration, first generation chemotherapeutic regimens for treatment of intermediate-grade non-Hodgkin's lymphoma (NHL) include C-MOPP (cyclophosphamide, vincristine, procarbazine and prednisone) and CHOP (cyclophosphamide, doxorubicin, vincristine, and prednisone). A useful second generation chemotherapeutic regimen is m-BACOD (methotrexate, bleomycin, doxorubicin, cyclophosphamide, vincristine, dexamethasone and leucovorin), while a suitable third generation regimen is MACOP-B (methotrexate, doxorubicin, cyclophosphamide, vincristine, prednisone, bleomycin and leucovorin). Additional useful drugs include phenyl butyrate, bendamustine, and bryostatin-1. In a preferred multimodal therapy, both chemotherapeutic drugs and cytokines are co-administered with an antibody complex. The cytokines, chemotherapeutic drugs and antibody complex can be administered in any order, or together.

Antibody complexes can be formulated according to known methods to prepare pharmaceutically useful compositions, whereby the antibody complex is combined in a mixture with a pharmaceutically suitable excipient. Sterile phosphate-buffered saline is one example of a pharmaceutically suitable excipient. Other suitable excipients are well-known to those in the art. See, for example, Ansel et al., PHARMACEUTICAL DOSAGE FORMS AND DRUG DELIVERY SYSTEMS, 5^{th} Edition (Lea & Febiger 1990), and Gennaro (ed.), REMINGTON'S PHARMACEUTICAL SCIENCES, 18^{th} Edition (Mack Publishing Company 1990), and revised editions thereof.

The antibody complex can be formulated for intravenous administration via, for example, bolus injection or continuous infusion. Preferably, the antibody complex is infused over a period of less than about 4 hours, and more preferably, over a period of less than about 3 hours. For example, the first 25-50 mg could be infused within 30 minutes, preferably even 15 min, and the remainder infused over the next 2-3 hrs. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

Additional pharmaceutical methods may be employed to control the duration of action of the antibody complex. Control release preparations can be prepared through the use of polymers to complex or adsorb the antibody complex. For example, biocompatible polymers include matrices of poly(ethylene-co-vinyl acetate) and matrices of a polyanhydride copolymer of a stearic acid dimer and sebacic acid. Sherwood et al., Bio/Technology 10: 1446 (1992). The rate of release of an antibody complex from such a matrix depends upon the molecular weight and the amount of the antibody complex within the matrix, and the size of dispersed particles. Saltzman et al., Biophys. J. 55: 163 (1989); Sherwood et al., supra. Other solid dosage forms are described in Ansel et al., PHARMACEUTICAL DOSAGE FORMS AND DRUG DELIVERY SYSTEMS, 5^{th} Edition (Lea & Febiger 1990), and Gennaro (ed.), REMINGTON'S PHARMACEUTICAL SCIENCES, 18^{th} Edition (Mack Publishing Company 1990), and revised editions thereof.

The antibody complex may also be administered to a mammal subcutaneously or even by other parenteral routes. Moreover, the administration may be by continuous infusion or by single or multiple boluses. Preferably, the antibody complex is infused over a period of less than about 4 hours, and more preferably, over a period of less than about 3 hours.

More generally, the dosage of an administered antibody complex for humans will vary depending upon such factors as the patient's age, weight, height, sex, general medical condition and previous medical history. It may be desirable to provide the recipient with a dosage of antibody complex that is in the range of from about 1 mg/kg to 25 mg/kg as a single intravenous infusion, although a lower or higher dosage also may be administered as circumstances dictate. A dosage of 1-20 mg/kg for a 70 kg patient, for example, is 70-1,400 mg, or 41-824 mg/m² for a 1.7-m patient. The dosage may be repeated as needed, for example, once per week for 4-10 weeks, once per week for 8 weeks, or once per week for 4 weeks. It may also be given less frequently, such as every other week for several months, or monthly or quarterly for many months, as needed in a maintenance therapy.

Alternatively, an antibody complex may be administered as one dosage every 2 or 3 weeks, repeated for a total of at least 3 dosages. Or, the antibody complex may be administered twice per week for 4-6 weeks. If the dosage is lowered to approximately 200-300 mg/m² (340 mg per dosage for a 1.7-m patient, or 4.9 mg/kg for a 70 kg patient), it may be administered once or even twice weekly for 4 to 10 weeks. Alternatively, the dosage schedule may be decreased, namely every 2 or 3 weeks for 2-3 months. It has been determined, however, that even higher doses, such as 20 mg/kg once weekly or once every 2-3 weeks can be administered by slow i.v. infusion, for repeated dosing cycles. The dosing schedule can optionally be repeated at other intervals and dosage may be given through various parenteral routes, with appropriate adjustment of the dose and schedule.

In preferred embodiments, the antibody complexes are of use for therapy of cancer. Examples of cancers include, but are not limited to, carcinoma, lymphoma, glioblastoma, melanoma, sarcoma, and leukemia, myeloma, or lymphoid malignancies. More particular examples of such cancers are noted below and include: squamous cell cancer (e.g., epithelial squamous cell cancer), Ewing sarcoma, Wilms tumor, astrocytomas, lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma multiforme, cervical cancer, ovarian cancer, liver cancer, bladder cancer, hepatoma, hepatocellular carcinoma, neuroendocrine tumors, medullary thyroid cancer, differentiated thyroid carcinoma, breast cancer, ovarian cancer, colon cancer, rectal cancer, endometrial cancer or uterine carcinoma, salivary gland carcinoma, kidney or renal cancer, prostate cancer, vulvar cancer, anal carcinoma, penile carcinoma, as well as head-and-neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Polycythemia vera, Parathyroid Cancer, Penile Cancer, Pheochromocytoma, Pituitary Tumor, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

The methods and compositions described and claimed herein may be used to treat malignant or premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, Basic Pathology, 2d Ed., W.B. Saunders Co., Philadelphia, pp. 68-79 (1976)). Such conditions in which cells begin to express, over-express, or abnormally express IGF-1R, are particularly treatable by the disclosed methods and compositions.

Dysplasia is frequently a forerunner of cancer, and is found mainly in the epithelia. It is the most disorderly form of non-neoplastic cell growth, involving a loss in individual cell uniformity and in the architectural orientation of cells. Dysplasia characteristically occurs where there exists chronic irritation or inflammation. Dysplastic disorders which can be treated include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders which can be treated include, but are not limited to, benign dysproliferative disorders (e.g., benign tumors, fibrocystic conditions, tissue hypertrophy, intestinal polyps or adenomas, and esophageal dysplasia), leukoplakia, keratoses, Bowen's disease, Farmer's Skin, solar cheilitis, and solar keratosis.

In preferred embodiments, the method of the invention is used to inhibit growth, progression, and/or metastasis of cancers, in particular those listed above.

Additional hyperproliferative diseases, disorders, and/or conditions include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendroglioma, meningioma, melanoma, neuroblastoma, and retinoblastoma.

Kits

Various embodiments may concern kits containing components suitable for treating or diagnosing diseased tissue in a patient. Exemplary kits may contain at least one or more PEGylated therapeutic agents as described herein. If the composition containing components for administration is not formulated for delivery via the alimentary canal, such as by oral delivery, a device capable of delivering the kit components through some other route may be included. One type of device, for applications such as parenteral delivery, is a syringe that is used to inject the composition into the body of a subject. Inhalation devices may also be used. In certain embodiments, a PEGylated therapeutic agent may be provided in the form of a prefilled syringe or autoinjection pen containing a sterile, liquid formulation or lyophilized preparation.

The kit components may be packaged together or separated into two or more containers. In some embodiments, the containers may be vials that contain sterile, lyophilized formulations of a composition that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers. Another component that can be included is instructions to a person using a kit for its use.

Dock and Lock (DNL) Method

In certain preferred embodiments, the multivalent, multispecific antibody complex may be produced using the dock-and-lock (DNL) technology (see, e.g., U.S. Pat. Nos. 7,521,056; 7,550,143; 7,534,866; 7,527,787 and 7,666,400; the Examples section of each of which is incorporated herein by reference). The DNL method exploits specific protein/protein interactions that occur between the regulatory \textcircled{R} subunits of cAMP-dependent protein kinase (PKA) and the anchoring domain (AD) of A-kinase anchoring proteins (AKAPs) (Baillie et al., FEBS Letters. 2005; 579: 3264. Wong and Scott, Nat. Rev. Mol. Cell Biol. 2004; 5: 959). PKA, which plays a central role in one of the best studied signal transduction pathways triggered by the binding of the second messenger cAMP to the R subunits, was first isolated from rabbit skeletal muscle in 1968 (Walsh et al., J. Biol. Chem. 1968; 243:3763). The structure of the holoenzyme consists of two catalytic subunits held in an inactive form by the R subunits (Taylor, J. Biol. Chem. 1989; 264:8443). Isozymes of PKA are found with two types of R subunits (RI and RII), and each type has α and β isoforms (Scott, Pharmacol. Ther. 1991; 50:123). Thus, the four isoforms of PKA regulatory subunits are RIα, RIβ, RIIα and RIIβ. The R subunits have been isolated only as stable dimers and the dimerization domain has been shown to consist of the first 44 amino-terminal residues (Newlon et al., Nat. Struct. Biol. 1999; 6:222). Binding of cAMP to the R subunits leads to the release of active catalytic subunits for a broad spectrum of serine/threonine kinase activities, which are oriented toward selected substrates through the compartmentalization of PKA via its docking with AKAPs (Scott et al., J. Biol. Chem. 1990; 265; 21561)

Since the first AKAP, microtubule-associated protein-2, was characterized in 1984 (Lohmann et al., Proc. Natl. Acad. Sci USA. 1984; 81:6723), more than 50 AKAPs that localize to various sub-cellular sites, including plasma membrane, actin cytoskeleton, nucleus, mitochondria, and endoplasmic reticulum, have been identified with diverse structures in species ranging from yeast to humans (Wong and Scott, Nat. Rev. Mol. Cell Biol. 2004; 5:959). The AD of AKAPs for PKA is an amphipathic helix of 14-18 residues (Carr et al., J. Biol. Chem. 1991; 266:14188). The amino acid sequences of the AD are quite varied among individual AKAPs, with the binding affinities reported for RII dimers ranging from 2 to 90 nM (Alto et al., Proc. Natl. Acad. Sci. USA. 2003; 100:4445). AKAPs will only bind to dimeric R subunits. For human RIIα, the AD binds to a hydrophobic surface formed by the 23 amino-terminal residues (Colledge and Scott, Trends Cell Biol. 1999; 6:216). Thus, the dimerization domain and AKAP binding domain of human RIIα are both located within the same N-terminal 44 amino acid sequence (Newlon et al., Nat. Struct. Biol. 1999; 6:222; Newlon et al., EMBO J. 2001; 20:1651), which is termed the DDD herein.

We have developed a platform technology to utilize the DDD of human RIIα and the AD of AKAP as an excellent pair of linker modules for docking any two entities, referred to hereafter as A and B, into a noncovalent complex, which could be further locked into a DNL complex through the introduction of cysteine residues into both the DDD and AD at strategic positions to facilitate the formation of disulfide bonds. The general methodology of the “dock-and-lock” approach is as follows. Entity A is constructed by linking a DDD sequence to a precursor of A, resulting in a first component hereafter referred to as a. Because the DDD sequence would effect the spontaneous formation of a dimer, A would thus be composed of a₂. Entity B is constructed by linking an AD sequence to a precursor of B, resulting in a second component hereafter referred to as b. The dimeric motif of DDD contained in a₂ will create a docking site for binding to the AD sequence contained in b, thus facilitating a ready association of a₂ and b to form a binary, trimeric complex composed of a₂b. This binding event is made irreversible with a subsequent reaction to covalently secure the two entities via disulfide bridges, which occurs very efficiently based on the principle of effective local concentration because the initial binding interactions should bring the reactive thiol groups placed onto both the DDD and AD into proximity (Chmura et al., Proc. Natl. Acad. Sci. USA. 2001; 98:8480) to ligate site-specifically. Using various combinations of linkers, adaptor modules and precursors, a wide variety of DNL constructs of different stoichiometry may be produced and used, including but not limited to dimeric, trimeric, tetrameric, pentameric and hexameric DNL constructs (see, e.g., U.S. Pat. Nos. 7,550,143; 7,521,056; 7,534,866; 7,527,787 and 7,666,400.)

By attaching the DDD and AD away from the functional groups of the two precursors, such site-specific ligations are also expected to preserve the original activities of the two precursors. This approach is modular in nature and potentially can be applied to link, site-specifically and covalently, a wide range of substances, including peptides, proteins, antibodies, antibody fragments, and other effector moieties with a wide range of activities. In some embodiments, the DNL complex may comprise two or more antibodies, antibody fragments or fusion proteins which bind to the same antigenic determinant or to two or more different antigens. The DNL complex may also comprise one or more other effectors, such as proteins, peptides, immunomodulators, cytokines, interleukins, interferons, binding proteins, peptide ligands, carrier proteins, toxins, ribonucleases such as onconase, inhibitory oligonucleotides such as siRNA, antigens or xenoantigens, polymers such as PEG, enzymes, therapeutic agents, hormones, cytotoxic agents, anti-angiogenic agents, pro-apoptotic agents or any other molecule or aggregate. Utilizing the fusion protein method of constructing AD and DDD conjugated effectors described in the Examples below, virtually any protein or peptide may be incorporated into a DNL construct. However, the technique is not limiting and other methods of conjugation may be utilized.

A variety of methods are known for making fusion proteins, including nucleic acid synthesis, hybridization and/or amplification to produce a synthetic double-stranded nucleic acid encoding a fusion protein of interest. Such double-stranded nucleic acids may be inserted into expression vectors for fusion protein production by standard molecular biology techniques (see, e.g. Sambrook et al., Molecular Cloning, A laboratory manual, 2^{nd} Ed, 1989). In such preferred embodiments, the AD and/or DDD moiety may be attached to either the N-terminal or C-terminal end of an effector protein or peptide. However, the skilled artisan will realize that the site of attachment of an AD or DDD moiety to an effector moiety may vary, depending on the chemical nature of the effector moiety and the part(s) of the effector moiety involved in its physiological activity. Site-specific attachment of a variety of effector moieties may be performed using techniques known in the art, such as the use of bivalent cross-linking reagents and/or other chemical conjugation techniques.

Structure-Function Relationships in AD and DDD Moieties

For different types of DNL constructs, different AD or DDD sequences may be utilized. Exemplary DDD and AD sequences are provided below.

DDD1
(SEQ ID NO: 1)
SHIQIPPGLTELLQGYTVEVLRQQPPDLVEFAVEYFTRLREARA
DDD2
(SEQ ID NO: 2)
CGHIQIPPGLTELLQGYTVEVLRQQPPDLVEFAVEYFTRLREARA
AD1
(SEQ ID NO: 3)
QIEYLAKQIVDNAIQQA
AD2
(SEQ ID NO: 4)
CGQIEYLAKQIVDNAIQQAGC

The skilled artisan will realize that DDD1 and DDD2 are based on the DDD sequence of the human RIIα isoform of protein kinase A. However, in alternative embodiments, the DDD and AD moieties may be based on the DDD sequence of the human RIα form of protein kinase A and a corresponding AKAP sequence, as exemplified in DDD3, DDD3C and AD3 below.

DDD3
(SEQ ID NO: 5)
SLRECELYVQKHNIQALLKDSIVQLCTARPERPMAFLREYFERLEKEEAK
DDD3C
(SEQ ID NO: 6)
MSCGGSLRECELYVQKHNIQALLKDSIVQLCTARPERPMAFLREYFERLE
KEEAK
AD3
(SEQ ID NO: 7)
CGFEELAWKIAKMIWSDVFQQGC

In other alternative embodiments, other sequence variants of AD and/or DDD moieties may be utilized in construction of the DNL complexes. For example, there are only four variants of human PKA DDD sequences, corresponding to the DDD moieties of PKA RIα, RIIα, RIβ and RIIβ. The RIIα DDD sequence is the basis of DDD1 and DDD2 disclosed above. The four human PKA DDD sequences are shown below. The DDD sequence represents residues 1-44 of RIIα, 1-44 of RIIβ, 12-61 of RIα and 13-66 of RIβ. (Note that the sequence of DDD1 is modified slightly from the human PKA RIIα DDD moiety.)

PKA RIα
(SEQ ID NO: 8)
SLRECELYVQKHNIQALLKDVSIVQLCTARPERPMAFLREYFEKLEKEEA
K
PKA RIβ
(SEQ ID NO: 9)
SLKGCELYVQLHGIQQVLKDCIVHLCISKPERPMKFLREHFEKLEKEENR
QILA
PKA RIIα
(SEQ ID NO: 10)
SHIQIPPGLTELLQGYTVEVGQQPPDLVDFAVEYFTRLREARRQ
PKA RIIβ
(SEQ ID NO: 11)
SIEIPAGLTELLQGFTVEVLRHQPADLLEFALQHFTRLQQENER

The structure-function relationships of the AD and DDD domains have been the subject of investigation. (See, e.g., Burns-Hamuro et al., 2005, Protein Sci 14:2982-92; Carr et al., 2001, J Biol Chem 276:17332-38; Alto et al., 2003, Proc Natl Acad Sci USA 100:4445-50; Hundsrucker et al., 2006, Biochem J 396:297-306; Stokka et al., 2006, Biochem J 400:493-99; Gold et al., 2006, Mol Cell 24:383-95; Kinderman et al., 2006, Mol Cell 24:397-408, the entire text of each of which is incorporated herein by reference.)

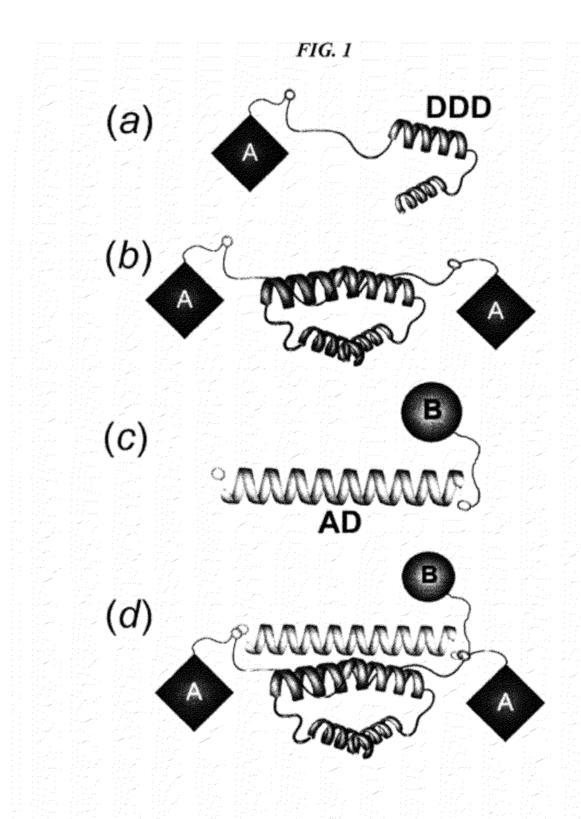

For example, Kinderman et al. (2006, Mol Cell 24:397-408) examined the crystal structure of the AD-DDD binding interaction and concluded that the human DDD sequence contained a number of conserved amino acid residues that were important in either dimer formation or AKAP binding, underlined in SEQ ID NO:1 below. (See FIG. 1 of Kinderman et al., 2006, incorporated herein by reference.) The skilled artisan will realize that in designing sequence variants of the DDD sequence, one would desirably avoid changing any of the underlined residues, while conservative amino acid substitutions might be made for residues that are less critical for dimerization and AKAP binding.

SHIQIPPGLTELLQGYTVEVLRQQPPDLVEFAVEYFTRLREARA (SEQ ID NO:1)

As discussed in more detail below, conservative amino acid substitutions have been characterized for each of the twenty common L-amino acids. Thus, based on the data of Kinderman (2006) and conservative amino acid substitutions, potential alternative DDD sequences based on SEQ ID NO:1 are shown in Table 3. In devising Table 3, only highly conservative amino acid substitutions were considered. For example, charged residues were only substituted for residues of the same charge, residues with small side chains were substituted with residues of similar size, hydroxyl side chains were only substituted with other hydroxyls, etc. Because of the unique effect of proline on amino acid secondary structure, no other residues were substituted for proline. Even with such conservative substitutions, there are over twenty million possible alternative sequences for the 44 residue peptide (2×3×2×2×2×2×2×2×2×2×2×2×2×2×2×4×2×2×2×2×2×4×2×4). A limited number of such potential alternative DDD moiety sequences are shown in SEQ ID NO:12 to SEQ ID NO:31 below. The skilled artisan will realize that an almost unlimited number of alternative species within the genus of DDD moieties can be constructed by standard techniques, for example using a commercial peptide synthesizer or well known site-directed mutagenesis techniques. The effect of the amino acid substitutions on AD moiety binding may also be readily determined by standard binding assays, for example as disclosed in Alto et al. (2003, Proc Natl Acad Sci USA 100:4445-50).

TABLE 3
Conservative Amino Acid Substitutions in DDD1 (SEQ ID NO: 1). Consensus
sequence disclosed as SEQ ID NO: 117.

| S | H | I | Q | I | P | P | G | L | T | E | L | L | Q | G | Y | T | V | E | V | L | R |
|---|---|---|---|---|---|---|---|---|---|---|---|---|---|---|---|---|---|---|---|---|---|
| T | K | | N | | | | A | | S | D | | | N | A | | S | | D | | | K |
| | R | | | | | | | | | | | | | | | | | | | | |
| Q | Q | P | P | D | L | V | E | F | A | V | E | Y | F | T | R | L | R | E | A | R | A |
| N | N | | | E | | | D | | L | | D | | | S | K | | K | D | L | K | L |
| | | | | | | | | | I | | | | | | | | | | I | | I |
| | | | | | | | | | V | | | | | | | | | | V | | V |

THIQIPPGLTELLQGYTVEVLRQQPPDLVEFAVEYFTRLREARA (SEQ ID NO: 12)
SKIQIPPGLTELLQGYTVEVLRQQPPDLVEFAVEYFTRLREARA (SEQ ID NO: 13)
SRIQIPPGLTELLQGYTVEVLRQQPPDLVEFAVEYFTRLREARA (SEQ ID NO: 14)
SHINIPPGLTELLQGYTVEVLRQQPPDLVEFAVEYFTRLREARA (SEQ ID NO: 15)
SHIQIPPALTELLQGYTVEVLRQQPPDLVEFAVEYFTRLREARA (SEQ ID NO: 16)
SHIQIPPGLSELLQGYTVEVLRQQPPDLVEFAVEYFTRLREARA (SEQ ID NO: 17)
SHIQIPPGLTDLLQGYTVEVLRQQPPDLVEFAVEYFTRLREARA (SEQ ID NO: 18)
SHIQIPPGLTELLNGYTVEVLRQQPPDLVEFAVEYFTRLREARA (SEQ ID NO: 19)
SHIQIPPGLTELLQAYTVEVLRQQPPDLVEFAVEYFTRLREARA (SEQ ID NO: 20)
SHIQIPPGLTELLQGYSVEVLRQQPPDLVEFAVEYFTRLREARA (SEQ ID NO: 21)
SHIQIPPGLTELLQGYTVDVLRQQPPDLVEFAVEYFTRLREARA (SEQ ID NO: 22)
SHIQIPPGLTELLQGYTVEVLKQQPPDLVEFAVEYFTRLREARA (SEQ ID NO: 23)
SHIQIPPGLTELLQGYTVEVLRNQPPDLVEFAVEYFTRLREARA (SEQ ID NO: 24)
SHIQIPPGLTELLQGYTVEVLRQNPPDLVEFAVEYFTRLREARA (SEQ ID NO: 25)
SHIQIPPGLTELLQGYTVEVLRQQPPELVEFAVEYFTRLREARA (SEQ ID NO: 26)
SHIQIPPGLTELLQGYTVEVLRQQPPDLVDFAVEYFTRLREARA (SEQ ID NO: 27)
SHIQIPPGLTELLQGYTVEVLRQQPPDLVEFLVEYFTRLREARA (SEQ ID NO: 28)
SHIQIPPGLTELLQGYTVEVLRQQPPDLVEFIVEYFTRLREARA (SEQ ID NO: 29)
SHIQIPPGLTELLQGYTVEVLRQQPPDLVEFVVEYFTRLREARA (SEQ ID NO: 30)
SHIQIPPGLTELLQGYTVEVLRQQPPDLVEFAVDYFTRLREARA (SEQ ID NO: 31)

Alto et al. (2003, Proc Natl Acad Sci USA 100:4445-50) performed a bioinformatic analysis of the AD sequence of various AKAP proteins to design an RII selective AD sequence called AKAP-IS (SEQ ID NO:3), with a binding constant for DDD of 0.4 nM. The AKAP-IS sequence was designed as a peptide antagonist of AKAP binding to PKA. Residues in the AKAP-IS sequence where substitutions tended to decrease binding to DDD are underlined in SEQ ID NO:3 below. The skilled artisan will realize that in designing sequence variants of the AD sequence, one would desirably avoid changing any of the underlined residues, while conservative amino acid substitutions might be made for residues that are less critical for DDD binding. Table 4 shows potential conservative amino acid substitutions in the sequence of AKAP-IS (AD1, SEQ ID NO:3), similar to that shown for DDD1 (SEQ ID NO:1) in Table 3 above.

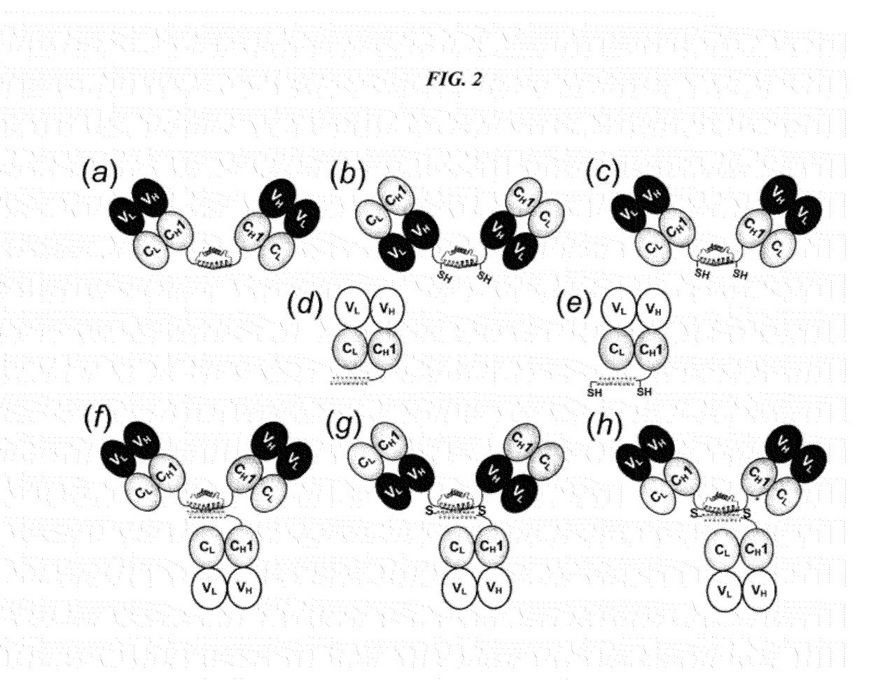

Even with such conservative substitutions, there are over thirty-five thousand possible alternative sequences for the 17 residue AD1 (SEQ ID NO:3) peptide sequence (2×3×2×4×3×2×2×2×2×2×2×4). A limited number of such potential alternative AD moiety sequences are shown in SEQ ID NO:32 to SEQ ID NO:49 below. Again, a very large number of species within the genus of possible AD moiety sequences could be made, tested and used by the skilled artisan, based on the data of Alto et al. (2003). It is noted that FIG. 2 of Alto (2003) shows an even large number of potential amino acid substitutions that may be made, while retaining binding activity to DDD moieties, based on actual binding experiments.

AKAP-IS
(SEQ ID NO: 3)
QIEYLAKQIVDNAIQQA

TABLE 4
Conservative Amino Acid Substitutions in AD1 (SEQ ID NO: 3).
Consensus sequence disclosed as SEQ ID NO: 118.

| Q | I | E | Y | L | A | K | Q | I | V | D | N | A | I | Q | Q | A |
|---|---|---|---|---|---|---|---|---|---|---|---|---|---|---|---|---|
| N | L | D | F | I | | R | N | | | E | Q | | | N | N | L |
| | V | | T | V | | | | | | | | | | | | I |
| | | | S | | | | | | | | | | | | | V |

NIEYLAKQIVDNAIQQA (SEQ ID NO: 32)
QLEYLAKQIVDNAIQQA (SEQ ID NO: 33)
QVEYLAKQIVDNAIQQA (SEQ ID NO: 34)
QIDYLAKQIVDNAIQQA (SEQ ID NO: 35)
QIEFLAKQIVDNAIQQA (SEQ ID NO: 36)
QIETLAKQIVDNAIQQA (SEQ ID NO: 37)
QIESLAKQIVDNAIQQA (SEQ ID NO: 38)
QIEYIAKQIVDNAIQQA (SEQ ID NO: 39)
QIEYVAKQIVDNAIQQA (SEQ ID NO: 40)
QIEYLARQIVDNAIQQA (SEQ ID NO: 41)
QIEYLAKNIVDNAIQQA (SEQ ID NO: 42)
QIEYLAKQIVENAIQQA (SEQ ID NO: 43)
QIEYLAKQIVDQAIQQA (SEQ ID NO: 44)
QIEYLAKQIVDNAINQA (SEQ ID NO: 45)
QIEYLAKQIVDNAIQNA (SEQ ID NO: 46)
QIEYLAKQIVDNAIQQL (SEQ ID NO: 47)
QIEYLAKQIVDNAIQQI (SEQ ID NO: 48)
QIEYLAKQIVDNAIQQV (SEQ ID NO: 49)

Gold et al. (2006, Mol Cell 24:383-95) utilized crystallography and peptide screening to develop a SuperAKAP-IS sequence (SEQ ID NO:50), exhibiting a five order of magnitude higher selectivity for the RII isoform of PKA compared with the RI isoform. Underlined residues indicate the positions of amino acid substitutions, relative to the AKAP-IS sequence, which increased binding to the DDD moiety of RIIα. In this sequence, the N-terminal Q residue is numbered as residue number 4 and the C-terminal A residue is residue number 20. Residues where substitutions could be made to affect the affinity for RIIα were residues 8, 11, 15, 16, 18, 19 and 20 (Gold et al., 2006). It is contemplated that in certain alternative embodiments, the SuperAKAP-IS sequence may be substituted for the AKAP-IS AD moiety sequence to prepare DNL constructs. Other alternative sequences that might be substituted for the AKAP-IS AD sequence are shown in SEQ ID NO:51-53. Substitutions relative to the AKAP-IS sequence are underlined. It is anticipated that, as with the AD2 sequence shown in SEQ ID NO:4, the AD moiety may also include the additional N-terminal residues cysteine and glycine and C-terminal residues glycine and cysteine.

SuperAKAP-IS
(SEQ ID NO: 50)
QIEYVAKQIVDYAIHQA
Alternative AKAP sequences
(SEQ ID NO: 51)
QIEYKAKQIVDHAIHQA
(SEQ ID NO: 52)
QIEYHAKQIVDHAIHQA
(SEQ ID NO: 53)
QIEYVAKQIVDHAIHQA

FIG. 2 of Gold et al. disclosed additional DDD-binding sequences from a variety of AKAP proteins, shown below.

RH-Specific AKAPs
AKAP-KL
(SEQ ID NO: 54)
PLEYQAGLLVQNAIQQAI
AKAP79
(SEQ ID NO: 55)
LLIETASSLVKNAIQLSI
AKAP-Lbc
(SEQ ID NO: 56)
LIEEAASRIVDAVIEQVK
RI-Specific AKAPs
AKAPce
(SEQ ID NO: 57)
ALYQFADRFSELVISEAL
RIAD
(SEQ ID NO: 58)
LEQVANQLADQIIKEAT
PV38
(SEQ ID NO: 59)
FEELAWKIAKMIWSDVF
Dual-Specificity AKAPs
AKAP7
(SEQ ID NO: 60)
ELVRLSKRLVENAVLKAV
MAP2D
(SEQ ID NO: 61)
TAEEVSARIVQVVTAEAV
DAKAP1
(SEQ ID NO: 62)
QIKQAAFQLISQVILEAT
DAKAP2
(SEQ ID NO: 63)
LAWKIAKMIVSDVMQQ

Stokka et al. (2006, Biochem J 400:493-99) also developed peptide competitors of AKAP binding to PKA, shown in SEQ ID NO:64-66. The peptide antagonists were designated as Ht31 (SEQ ID NO:64), RIAD (SEQ ID NO:65) and PV-38 (SEQ ID NO:66). The Ht-31 peptide exhibited a greater affinity for the RII isoform of PKA, while the RIAD and PV-38 showed higher affinity for RI.

Ht31
(SEQ ID NO: 64)
DLIEEAASRIVDAVIEQVKAAGAY
RIAD
(SEQ ID NO: 65)
LEQYANQLADQIIKEATE
PV-38
(SEQ ID NO: 66)
FEELAWKIAKMIWSDVFQQC

Hundsrucker et al. (2006, Biochem J 396:297-306) developed still other peptide competitors for AKAP binding to PKA, with a binding constant as low as 0.4 nM to the DDD of the RH form of PKA. The sequences of various AKAP antagonistic peptides are provided in Table 1 of Hundsrucker et al., reproduced in Table 5 below. AKAPIS represents a synthetic RII subunit-binding peptide. All other peptides are derived from the RII-binding domains of the indicated AKAPs.

TABLE 5
AKAP Peptide sequences

| | Peptide Sequence |
|---|---|
| AKAPIS | QIEYLAKQIVDNAIQQA |
| | (SEQ ID NO: 3) |
| AKAPIS-P | QIEYLAKQIPDNAIQQA |
| | (SEQ ID NO: 67) |
| Ht31 | KGADLIEEAASRIVDAVIEQVKAAG |
| | (SEQ ID NO: 68) |
| Ht31-P | KGADLIEEAASR1PDAPIEQVKAAG |
| | (SEQ ID NO: 69) |
| AKAP7δ-wt-pep | PEDAELVRLSKRLVENAVLKAVQQY |
| | (SEQ ID NO: 70) |
| AKAP7δ-L304T-pep | PEDAELVRTSKRLVENAVLKAVQQY |
| | (SEQ ID NO: 71) |
| AKAP7δ-L308D-pep | PEDAELVRLSKRDVENAVLKAVQQY |
| | (SEQ ID NO: 72) |
| AKAP7δ-P-pep | PEDAELVRLSKRLPENAVLKAVQQY |
| | (SEQ ID NO: 73) |
| AKAP7δ-PP-pep | PEDAELVRLSKRLPENAPLKAVQQY |
| | (SEQ ID NO: 74) |
| AKAP7δ-L314E-pep | PEDAELVRLSKRLVENAVEKAVQQY |
| | (SEQ ID NO: 75) |
| AKAP1-pep | EEGLDRNEEIKRAAFQIISQVISEA |
| | (SEQ ID NO: 76) |
| AKAP2-pep | LVDDPLEYQAGLLVQNAIQQAIAEQ |
| | (SEQ ID NO: 77) |
| AKAP5-pep | QYETLLIETASSLVKNAIQLSIEQL |
| | (SEQ ID NO: 78) |
| AKAP9-pep | LEKQYQEQLEEEVAKVIVSMSIAFA |
| | (SEQ ID NO: 79) |
| AKAP10-pep | NTDEAQEELAWKIAKMIVSDIMQQA |
| | (SEQ ID NO: 80) |
| AKAP11-pep | VNLDKKAVLAEKIVAEAIEKAEREL |
| | (SEQ ID NO: 81) |
| AKAP12-pep | NGILELETKSSKLVQNIIQTAVDQF |
| | (SEQ ID NO: 82) |
| AKAP14-pep | TQDKNYEDELTQVALALVEDVINYA |
| | (SEQ ID NO: 83) |
| Rab32-pep | ETSAKDNINIEEAARFLVEKILVNH |
| | (SEQ ID NO: 84) |

Residues that were highly conserved among the AD domains of different AKAP proteins are indicated below by underlining with reference to the AKAP IS sequence (SEQ ID NO:3). The residues are the same as observed by Alto et al. (2003), with the addition of the C-terminal alanine residue. (See FIG. 8 of Hundsrucker et al. (2006), incorporated herein by reference.) The sequences of peptide antagonists with particularly high affinities for the RII DDD sequence were those of AKAP-IS, AKAP7δ-wt-pep, AKAP78-L304T-pep and AKAP75-L308D-pep.

AKAP-IS

QIEYLAKQIVDNAIQQA (SEQ ID NO:3)

Carr et al. (2001, J Biol Chem 276:17332-38) examined the degree of sequence homology between different AKAP-binding DDD sequences from human and non-human proteins and identified residues in the DDD sequences that appeared to be the most highly conserved among different DDD moieties. These are indicated below by underlining with reference to the human PKA RIIα DDD sequence of SEQ ID NO:1. Residues that were particularly conserved are further indicated by italics. The residues overlap with, but are not identical to those suggested by Kinderman et al. (2006) to be important for binding to AKAP proteins. The skilled artisan will realize that in designing sequence variants of DDD, it would be most preferred to avoid changing the most conserved residues (italicized), and it would be preferred to also avoid changing the conserved residues (underlined), while conservative amino acid substitutions may be considered for residues that are neither underlined nor italicized.

SHIQIPPGLTELLQGYTVEVLRQQPPDLVEFAVEYFTRLREARA (SEQ ID NO:1)

A modified set of conservative amino acid substitutions for the DDD 1 (SEQ ID NO:1) sequence, based on the data of Carr et al. (2001) is shown in Table 6. Even with this reduced set of substituted sequences, there are over 65,000 possible alternative DDD moiety sequences that may be produced, tested and used by the skilled artisan without undue experimentation. The skilled artisan could readily derive such alternative DDD amino acid sequences as disclosed above for Table 3 and Table 4.

TABLE 6
Conservative Amino Acid Substitutions in DDD1 (SEQ ID NO: 1).
Consensus sequence disclosed as SEQ ID NO: 119.

| S | H | I | Q | [symbol] | [symbol] | P | [symbol] | [symbol] | T | E | [symbol] | [symbol] | Q | [symbol] | [symbol] | [symbol] | V | [symbol] | [symbol] | [symbol] | [symbol] |
|---|---|---|---|---|---|---|---|---|---|---|---|---|---|---|---|---|---|---|---|---|---|
| T | | | N | | | | | | S | | | | | | | | I | | | | |
| | | | | | | | | | | | | | | | | | L | | | | |
| | | | | | | | | | | | | | | | | | A | | | | |
| Q | [symbol] | [symbol] | P | [symbol] | [symbol] | V | E | [symbol] | [symbol] | V | E | [symbol] | [symbol] | T | R | [symbol] | R | E | A | [symbol] | A |
| N | | | | | | | | | | I | D | | | S | K | | K | | L | | L |
| | | | | | | | | | | L | | | | | | | | | I | | I |
| | | | | | | | | | | A | | | | | | | | | V | | V |

The skilled artisan will realize that these and other amino acid substitutions in the DDD or AD amino acid sequences may be utilized to produce alternative species within the genus of AD or DDD moieties, using techniques that are standard in the field and only routine experimentation.

scFv-Based AD Modules

Alternative embodiments may concern the use of scFv-based AD modules for pairing with DDD2 (SEQ ID NO:3) conjugated antibodies or antibody fragments to yield DNL conjugates that contain multiple binding sites for any selected combination of antigens. We have produced several types of scFv-based bispecific antibodies by expressing two discrete polypeptide chains comprising complementary variable domains with a 6-His tag at the carboxyl terminus of each polypeptide chain. The same approach may be used to generate scFv-based AD modules by replacing one or both 6-His tags with either an AD sequence or an AD-6-His sequence. We can also fuse each polypeptide chain with a different AD sequence (e.g. AD2 (SEQ ID NO:4) and AD3 (SEQ ID NO:7)), which would allow the specific recognition by its cognate DDD sequence, thus providing further complexity of the final DNL conjugates. Table 7 below provides a non-exhaustive list of such scFv-based DNL constructs.

TABLE 7
scFv-based DNL constructs

| Configuration | | ScFv-AD | Note |
|---|---|---|---|
| BS2 | I | VH₁-VL₂-AD2 | Bispecific, 1 × 1 |
| | | VH₂-VL₁ | |
| | II | VH₁-VL₂-AD2 | |
| | | VH₂-VL₁-AD2 | |
| | III | VH₁-VL₂-AD2 | |
| | | VH₂-VL₁-AD3 | |
| “DVD” | I | VH₁-VH₂-AD2 | Bispecific, 1 × 1 |
| | | VL₁-VL₂ | |
| | II | VH₁-VH₂-AD2 | |
| | | VL₂-VL₁-AD2 | |
| | III | VH₁-VH₂-AD2 | |
| | | VL₂-VL₁-AD3 | |
| BS6 | I | VH₁-VL₁-VH₂-AD2 | Bispecific, 2 × 1 |
| | | VL₂-VH₁-VL₁ | |
| | II | VH₁-VL₁-VH₂-AD2 | |
| | | VL₂-VH₁-VL₁-AD2 | |
| | III | VH₁-VL₁-VH₂-AD2 | |
| | | VL₂-VH₁-VL₁-AD3 | |
| BS8 | I | VH₁-VH₁-VH₂-AD2 | Bispecific, 2 × 1 |
| | | VL₂-VL₁-VL₁ | |
| | II | VH₁-VH₁-VH₂-AD2 | |
| | | VL₂-VL₁-VL₁-AD2 | |
| | III | VH₁-VH₁-VH₂-AD2 | |
| | | VL₂-VL₁-VL₁-AD3 | |
| BS18 | I | VH₁-CH₁-VH₂-AD2 | |
| | | VL₁-CL-VL₂ | |
| | II | VH₁-CH₁-VH₂-AD2 | |
| | | VL₁-CL-VL₂-AD2 | |
| | III | VH₁-CH₁-VH₂-AD2 | |
| | | VL₁-CL-VL₂-AD3 | |
| TS | I | VH₁-VH₂-VH₃-AD2 | Trispecific, 1 × 1 × 1 |
| | | VL₃-VL₂-VL₁ | |
| | II | VH₁-VH₂-VH₃-AD2 | |
| | | VL₃-VL₂-VL₁-AD2 | |
| | III | VH₁-VH₂-VH₃-AD2 | |
| | | VL₃-VL₂-VL₁-AD3 | |

Type I is designed to link one pair of DDD2 (SEQ ID NO:2) modules. Type II is designed to link two pairs of the same or different DDD2 modules. Type III is designed to link one pair of DDD2 modules and one pair of DDD3 (SEQ ID NO:5) modules. The two polypeptides chains are designed to associate in an anti-parallel fashion.

Amino Acid Substitutions

In certain embodiments, the disclosed methods and compositions may involve production and use of proteins or peptides with one or more substituted amino acid residues. The skilled artisan will be aware that, in general, amino acid substitutions typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within ±2 is preferred, within ±1 are more preferred, and within ±0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg® gln, asn, lys; Asn (N) his, asp, lys, arg, gln; Asp (D) asn, glu; Cys (C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See, e.g., PROWL website at rockefeller.edu) For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (Id.) Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded protein sequence are well known and a matter of routine experimentation for the skilled artisan, for example by the technique of site-directed mutagenesis or by synthesis and assembly of oligonucleotides encoding an amino acid substitution and splicing into an expression vector construct.

Expression Vectors

Still other embodiments may concern DNA sequences comprising a nucleic acid encoding an antibody or fusion protein. Fusion proteins may comprise an antibody attached to a different peptide or protein, such as an scFv moiety or the AD and DDD peptides utilized for DNL construct formation.

Various embodiments relate to expression vectors comprising the coding DNA sequences. The vectors may contain sequences encoding the light and heavy chain constant regions and the hinge region of a human immunoglobulin to which may be attached chimeric, humanized or human variable region sequences. The vectors may additionally contain promoters that express the encoded protein(s) in a selected host cell, enhancers and signal or leader sequences. Vectors that are particularly useful are pdHL2 or GS. More preferably, the light and heavy chain constant regions and hinge region may be from a human EU myeloma immunoglobulin, where optionally at least one of the amino acid in the allotype positions is changed to that found in a different IgG1 allotype, and wherein optionally amino acid 253 of the heavy chain of EU based on the EU number system may be replaced with alanine. See Edelman et al., Proc. Natl. Acad. Sci USA 63: 78-85 (1969).

The skilled artisan will realize that methods of genetically engineering expression constructs and insertion into host cells to express engineered proteins are well known in the art and a matter of routine experimentation. Host cells and methods of expression of cloned antibodies or fragments have been described, for example, in U.S. Pat. Nos. 7,531,327; 7,537,930 and 7,608,425, the Examples section of each incorporated herein by reference.

### EXAMPLES

### Example 1

### Multifunctional, Multivalent Antibodies and Immunoconjugates Made by the Dock-and-Lock (DNL) Technique

Advances in new technologies for both rational and combinatorial protein engineering have increased the variety and magnitude of potential molecules that may be designed and produced for biotechnological and biomedical applications. An essential factor for these accomplishments is that many natural proteins have their functional properties located in discrete domains, which can be manipulated as molecular modules and exploited as building blocks for devising artificial structures with multiple functions. Molecular modules derived from the binding domains of antibodies or non-antibodies, or those based on protein display scaffolds, currently have received the most attention. Molecular modules that confer effector functions of proteins, for example, the Fc of an IgG or the catalytic domain of an enzyme, also constitute an important class of components in the architecture of designer proteins. In addition, peptide motifs with the innate ability to self-associate are often built into molecular modules to facilitate the formation of dimeric, trimeric or multimeric products composed of the same or different fusion polypeptides. Nevertheless, we have recognized that innovative fusion proteins created by recombinant engineering with these molecular modules may be built into more complex structures to gain additional attributes that are highly desirable, yet not technically attainable, in the individual engineered construct. To date, such goals are commonly achieved with varied success by judicious application of conjugation chemistries. Well known examples include PEGylated cytokines to increase serum half-lives (Pepinsky et al., 2001, J Pharmacol. Exp. Ther. 297:1059-1066), biotinylated proteins to enable immobilization into microarrays (Pepinsky et al., 2001, J Pharmacol. Exp. Ther. 297:1059-1066; Tan et al., 2004, Bioorg. Med. Chem. Lett., 14:6067-6070), and intein-mediated assembly of protein-DNA chimeras to quantify specific molecules to which the protein binds (Burbulis et al., 2005, Nat. Methods 2:31-37).

Relatively new to the bioconjugate field are strategies for tethering two or more molecular modules of distinct functions into covalent or quasi-covalent assemblies following a binding event. We present here an overview of one such strategy known as dock-and-lock (DNL), which can make a wide variety of bioactive molecules with multivalency, multifunctionality, and defined composition.

DDD/AD-Modules Based on PKA and AKAP

The basis of the DNL method is the exploitation of the specific protein/protein interactions occurring in nature between the regulatory (R) subunits of cAMP-dependent protein kinase PKA and the anchor domain (AD) of A-kinase anchor proteins (AKAPs), as discussed in the section above on Dock and Lock (DNL) Method.

In the DNL method, AD and DDD peptide sequences, which are modified with cysteine residues for covalent “locking” via disulfide bridges, are fused to a precursor protein (or other entity) to make DNL-modules, which are produced and stored independently (FIGS. 1a and 1c). The DDD-derivatized proteins (DDD-modules) spontaneously form stable homodimers; therefore, DDD-modules always comprise two copies of the precursor protein (FIG. 1b). Any DDD-module can be paired with any AD-module to generate a wide variety of stable conjugates comprising two copies of the DDD-module and one copy of the AD2-module precursors (FIG. 1d).

As discussed in the Examples below, we showed that the DDD of human PKA RIIα (designated DDD1) and the AD derived from AKAP-IS (Alto et al., 2003, Proc Natl Acad Sci U.S.A, 100:4445-4450) a synthetic peptide optimized for RII-selective binding with a reported K_{D} of 4×10⁻¹⁰ M (designated AD1), to be an excellent pair of linker peptides for docking two entities into a noncovalent complex, which could be further locked into a stably-tethered structure through the introduction of cysteine residues into both the DDD and AD at strategic positions to facilitate the formation of disulfide bonds. However, the cysteine-modified versions of AD1 and DDD1, designated AD2 and DDD2, allow stabilization of the DNL complex by covalent disulfide bond formation and are preferred. Advantages of the DNL technique for complex formation are summarized below.

DNL is Modular.

Each DDD- or AD-containing entity serves as a module and any DDD-module can be paired with any AD-module. Such modules can be produced independently, stored separately “on shelf”, and combined on demand. There is essentially no limit on the types of precursors that can be converted into a DDD- or AD-module, so long as the resulting modules do not interfere with the dimerization of DDD or the binding of DDD to AD.

DNL is Versatile.

Modules can be made recombinantly or synthetically. Recombinant modules, which may be produced in mammalian, microbial or other expression systems, may include fusions of antibodies or antibody fragments, cytokines, enzymes, carrier proteins (e.g., human serum albumin and human transferrin), or a variety of natural or artificial non-antibody binding or scaffold proteins. Furthermore, DDD or AD moieties can be coupled to the amino-terminal or carboxyl terminal end or even positioned internally within the fusion protein, preferably with a spacer containing an appropriate length and composition of amino acid residues, provided that the binding activity of the DDD or AD and the desired activity of the polypeptide fusion partners are not compromised. Two or more AD peptides can be incorporated into AD modules to create a scaffold for attachment of multiple DDD modules.

Modules can also be made synthetically, to comprise peptides, polyethylene glycol (PEG), dendrimers, nucleic acids, chelators with or without radioactive or non-radioactive metals, drugs, dyes, oligosaccharides, natural or synthetic polymeric substances, nanoparticles, fluorescent molecules, or quantum dots.

DNL Manufacturing is Easy.

The DNL method is basically a one-pot preparation and requires three simple steps to recover the product from the starting materials: (i) combine DDD- and AD-modules in stoichiometric amounts; (ii) add redox agents to facilitate the self-assembly of the DNL-conjugate; and (iii) purify by an appropriate affinity chromatography process. The DNL-modules can be purified and stored prior to their use in a DNL conjugation. However, purification of the modules is not necessary. DNL conjugation can be accomplished in mixtures of cell lysates and/or culture supernatant fluids containing the DNL-modules, with subsequent isolation of the DNL-conjugate by affinity chromatography. A single-step affinity purification process with commonly used affinity media, such as Protein A or immobilized metal, typically results in >95% purity of the DNL conjugate, which is sufficient for most pre-clinical applications. However, for manufacturing of clinical material, further processing steps, such as additional affinity chromatography, ion exchange chromatography, low pH treatment and ultrafiltration, ensure adequate removal of virus and other contaminants.

DNL Results in Quantitative Yields of a Homogeneous Product with a Defined Composition and In Vivo Stability.

The high-affinity binding between the DDD- and AD-modules results in nearly 100% conversion of each into the desired DNL product. The site-specific conjugation results in a preparation of defined and homogeneous molecular size and composition, for which the full activity of each module is usually preserved and in vivo integrity is sustained.

As discussed in the Examples below, the DNL method was applied to generate bispecific trivalent complexes comprising three Fab fragments by combining DDD-modules constructed from the Fab of hMN-14, a humanized monoclonal antibody (mAb) with specificity for the A3B3 domains of human carcinoembryonic antigen (CEACAM5), with AD-modules constructed from the Fab of h679, a humanized mAb with specificity for the hapten histamine-succinyl-glycine (HSG).

Three Fab-DDD-modules and two Fab-AD2-modules were used to generate three different Tri-Fabs (TFs), each comprising two hMN-14 Fabs and one h679 Fab. Fab-based modules are purified by affinity chromatography using Protein L or KappaSelect affinity media. C_{H}1-DDD1-Fab-hMN-14 (FIG. 2a) and C_{H}1-AD1-Fab-h679 (FIG. 2d) are Fab-DDD- and Fab-AD-modules of the respective parent mAbs, fused at the carboxyl-terminal end of the F_{d} chain (C-terminal end of C_{H}1 domain) to DDD1 and AD1, respectively. Upon mixing of the two modules, the formation of a binary complex (FIG. 2f) was readily demonstrated by SE-HPLC with the K_{D} determined by equilibrium gel filtration analysis to be about 8 nM, which is presumably too weak of an affinity for in-vivo applications. DDD1 was converted to DDD2 by incorporation of a cysteine residue at the amino-terminal end of the DDD peptide. Thus, the naturally dimeric DDD2-modules have two reactive cysteine residues. Two additional modules were generated for the hMN-14 Fab, N-DDD2-Fab-hMN-14 (FIG. 2b) and C_{H}1-DDD2-Fab-hMN-14 (FIG. 2c), where the DDD2 peptide was fused to the amino- or carboxyl-terminal end of the F_{d} chain, respectively. A cysteine residue was added to each end of AD1 to create AD2, which was included in the C_{H}1-AD2-Fab-h679 module (FIG. 2e). Two stably-tethered trivalent bispecific structures, referred to as TF1 (FIG. 2g) for the conjugate of N-DDD2-Fab-hMN-14 and C_{H}1-AD2-Fab-h679 and TF2 (FIG. 2h) for the conjugate of C_{H}1-DDD2-Fab-hMN-14 and C_{H}1-AD2-Fab-h679, were produced in nearly quantitative yields and characterized extensively. TF1 and TF2 were isolated by affinity chromatography using the h679-binding hapten HSG, and were each resolved by SE-HPLC as a single peak of the expected molecular size (˜150 kDa). BIACORE™ demonstrated bispecific binding, which was confirmed by competition ELISA to be equivalent to hMN-14 IgG and h679 Fab, reflecting the retention of valency and binding affinity. Furthermore, TF1 and TF2 were stable for at least 7 days at 37° C. in human or mouse serum. The superiority of TF2 as a pretargeting agent for diagnostic imaging has been demonstrated in numerous studies (Schoffelen et al., 2010, J Nucl Med, 51:1780-1787; Sharkey et al., 2010, Semin Nucl Med 40:190-203; McBride et al., 2009, J Nucl Med 50:991-998; Sharkey et al., 2008, Radiology 246:497-507).

Since the generation of TF1 and TF2, the modular DNL method has allowed the rapid development of over 20 different trivalent bispecific Fab-based complexes by combinatorial pairing of monomeric Fab-AD2 and dimeric Fab-DDD2 modules (Gold et al., 2008, Cancer Res 68:4819-4826; Goldenberg et al., 2008, J Nucl Med 49:158-163; Karacay et al., 2009, J Nucl Med 50:2008-2016; Karacay et al., 2011, J Nucl Med 52:555-559, Schoffelen et al., 2010, J Nucl Med 51:1780-1787; Sharkey et al., 2007, Clin Cancer Res 13:5577s-5585s; Sharkey et al., 2008, Cancer Res 68:5282-5290, Sharkey et al., 2009, J Nucl Med 50:444-453; Sharkey et al., 2010, Semin Nucl Med 40:190-203; Sharkey et al., 2010, Cancer Biother Radiopharm 25:1-12). The technology platform has also been expanded to generate a variety of conjugates, including multivalent, multifunctional antibodies and immunocytokines, which are highlighted below.

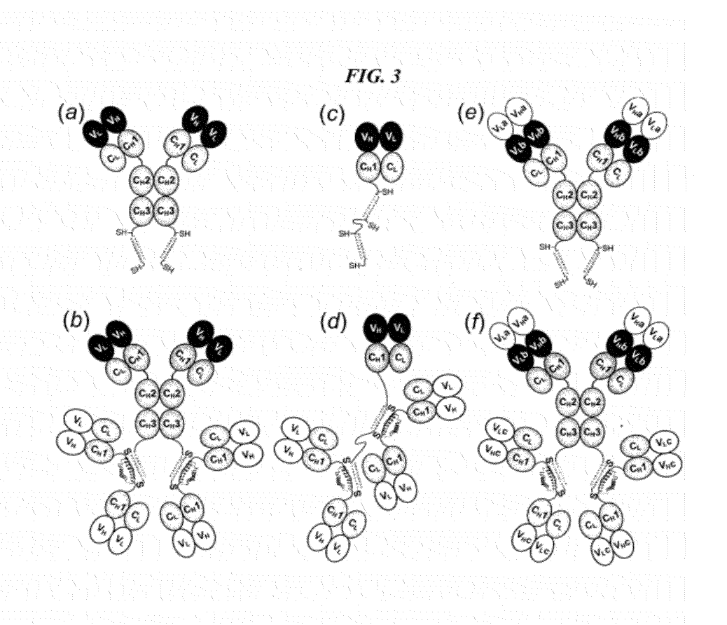

As discussed in the Examples below, we developed AD2-modules for IgG, which allowed the synthesis of a variety of complex, IgG-based DNL conjugates. C_{H}3-AD2-IgG modules were produced recombinantly by appending, in frame, the coding sequence for AD2, preceded by a flexible peptide linker, to the 3′ end of the coding sequence of the C_{H}3 domain of IgG (FIG. 3a). This method allowed the simple conversion of any existing IgG-expression plasmid vector into one for IgG-AD2 expression. Because IgG naturally forms a heterotetramer comprising two heavy and two light chains, IgG-AD2-modules possess two AD2 peptides, having one on the carboxyl-terminal end of each heavy chain (FIG. 3a). The IgG-AD2-modules were purified similar to IgG, using Protein A affinity chromatography. Each AD2 binds a dimeric X-DDD2 module, resulting in defined DNL structures comprising an IgG fused at its carboxyl terminus to four X groups, where X denotes an entity that could be a protein, peptide, polymer, drug or other molecule.

The IgG-AD2-modules were used to construct hexavalent antibodies (HexAbs), which were produced by combination of IgG-AD2 with the same Fab-DDD2 modules used in the Tri-Fab series, highlighting the advantage of the modular nature of DNL. Initially, monospecific HexAbs were produced using IgG-AD2- and Fab-DDD2-modules derived from the same parental mAb. As an example, a HexAb was made for the humanized anti-CD20 mAb veltuzumab (hA20), where C_{H}3-AD2-IgG-hA20 was combined with C_{H}1-DDD2-Fab-hA20. The DNL conjugation resulted in a homogeneous preparation of a conjugate comprising an F_{c} and 6 functional anti-CD20 Fabs, which each retained the binding affinity of veltuzumab. The construct, originally named Hex-hA20, has been designated 20-(20)-(20), using a standardized naming system where the first code indicates the IgG-AD2-module and the codes in parentheses indicate dimeric Fab-DDD2-modules.

Compared to veltuzumab, 20-(20)-(20) exhibited 3-fold higher binding avidity by ELISA, and a 3-fold slower off-rate from live NHL cells by flow cytometry, which demonstrated that all 6 binding arms can bind CD20 on cells. In vitro, 20-(20)-(20) inhibited proliferation of NHL cells at subnanomolar concentrations without the need for an additional crosslinking antibody. For 20-(20)-(20), some of the Fc-associated effector functions, including antibody-dependent cell-mediated cytotoxicity (ADCC), were retained, while others were apparently compromised. Unlike veltuzumab, 20-(20)-(20) did not effect complement-dependent cytotoxicity (CDC) in vitro. However, it is unclear if this is also the case in vivo, because recent experimental evidence suggests CDC mediated by similarly constructed HexAbs in whole blood. Even though 20-(20)-(20) is a nearly 2.5-fold larger molecule than veltuzumab, the circulating serum half-life is shorter for the former, suggesting that its interaction with the neonatal F_{c} receptor is altered. Although 20-(20)-(20) has reduced circulating half-life and CDC, it still demonstrated anti-tumor efficacy, which was comparable to veltuzumab at equivalent doses, in tumor-bearing mice.

Bispecific hexavalent antibodies (bsHexAbs) are readily constructed using DNL by combining an IgG-AD2 module with a Fab-DDD2 module derived from a different parental mAb (FIG. 3b). A large variety of bsHexAbs can be generated from a relatively small number of DNL modules. For example 100 different bsHexAbs can be made combinatorially using ten IgG-AD2 and ten Fab-DDD2 modules.

As discussed in the Examples below, the first bsHexAbs produced were derived from the humanized anti-CD22 mAb epratuzumab (hLL2) and veltuzumab. Combination of C_{H}3-AD2-IgG-hA20 with C_{H}1-DDD2-Fab-hLL2 produced 20422)-(22), which comprised veltuzumab with four Fabs of epratuzumab. A bsHexAb of the opposite configuration, 22-(20)-(20), which has four Fabs of veltuzumab fused to epratuzumab, was generated from C_{H}3-AD2-IgG-hLL2 and C_{H}1-DDD2-Fab-hA20. Characterization of the bsHexAbs demonstrated that the DNL conjugation resulted in highly purified covalent structures of the expected size and composition. Both 22-(20)-(20) and 20-(22)-(22) retained the binding properties of their parental Fab/IgGs with all 6 Fabs apparently capable of binding simultaneously. The bsHexAbs exhibited biological activities that were not observed using a mixture of the parental mAbs. Treatment of cells with the balexAbs, but not veltuzumab plus epratuzumab, resulted in translocation of both CD22 and CD20 into lipid rafts, induction of apoptosis and growth inhibition without second-antibody crosslinking, and homotypic adhesion. The bsHexAbs induced significant increases in the levels of phosphorylated p38 and PTEN, and also notable differences in signaling events from those incurred by crosslinking veltuzumab or rituximab with a secondary antibody (Gupta et al., 2010, Blood, 116:3258-3267). Thus, the greatly enhanced direct toxicity of these bsHexAbs correlated with their ability to alter the basal expression of various intracellular proteins involved in regulating cell growth, survival, and apoptosis, with the net outcome leading to cell death. Indeed, the bsHexAbs killed lymphoma cells in vitro much more potently than the parental mAb mixture.

As observed previously for the monospecific HexAbs, both bsHexAbs exhibited ADCC, but not CDC (in vitro), and had shorter circulating half-lives than the parent mAbs. Intriguingly, 22-(20)-(20) and 20422)-(22) killed human lymphoma cells in preference to normal B cells in whole human blood (ex vivo), whereas the parental veltuzumab depleted malignant and normal B cells about equally. In-vivo studies with NHL xenografts revealed 20-(22)-(22), despite having a shorter serum half-life, had anti-tumor efficacy comparable to veltuzumab. The 22-(20)-(20) was less potent than 20-(22)-(22), but more effective than epratuzumab and control bsAbs.

We have produced many bispecific Tri-Fab and HexAb DNL conjugates, which are of 1×2 and 2×4 Fab formats, respectively. In addition, we have produced trispecific HexAbs (2×2×2) by combining two different Fab-DDD2-modules with an IgG-AD2-module of a third specificity. A wide variety of alternative formats of multispecific antibodies is attainable with the introduction of new types of DNL-modules. For example, we have produced a Fab-based module with tandem AD2 peptides fused to the carboxyl-terminal end of the F_{d} (FIG. 3c). Combination with a Fab-DDD2 module resulted in a 1×4 bispecific conjugate comprising five Fabs, without an F_{c} (FIG. 3d), demonstrating that multiple AD2s can be incorporated into a single module. Application of this concept to an IgG module (IgG-AD2-AD2) would allow the creation of a multispecific IgG having a total of 10 Fabs, via combination with existing Fab-DDD2 modules. AD2- and DDD2-modules could be generated for other types of antibody-based proteins/fragments including scFᵥ, diabody, minibody, dual variable domain IgG, etc., providing a myriad of possible multispecific antibody formats. As a final example, we have generated a bispecific dual variable domain IgG-AD2 module (FIG. 3e), which was combined with a Fab-DDD2 module to generate a 2×2×4 trispecific octavalent antibody (FIG. 3f).

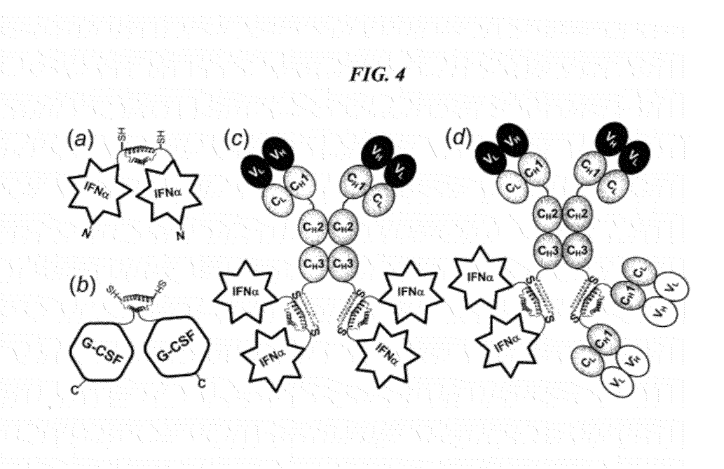

As discussed in the Examples below, DNL is not restricted to antibody-based constructs, as recombinant DDD2 or AD2 modules can be constructed for almost any protein. We have applied the DNL method to various cytokines, including interferon-alpha (IFNα), erythropoietin, and granulocyte colony-stimulating factor (G-CSF), to generate various immunocytokines (FIG. 4). The cytokine modules were produced in either E. coli or mammalian cell culture with AD2 or DDD2 fused at their amino- or carboxyl-terminal ends (FIGS. 4a and 4b). We have utilized a DDD2-module of human protamine which, when paired with an IgG-AD2 module, can be used for targeted cellular delivery of nucleic acids, such as siRNA. As an example of a module of a precursor that is both an enzyme and a toxin, a DDD2-module of ranpirnase (Rap, a cytotoxic ribonuclease from Rana pipiens) has been combined with various IgG-AD2 modules for targeted delivery of the toxin to tumor cells. In addition to recombinant protein modules, non-protein DNL-modules can be assembled using synthetic AD2 peptides coupled to a variety of synthetic precursor molecules (e.g., Chang et al., 2009, Bioconjug Chem 20:1899-1907).

Our most utilized cytokine module, IFNα2b-DDD2, which is produced with recombinant myeloma cell culture, has a carboxyl-terminal DDD appended to IFNα2b (FIG. 4a). We have combined IFNα2b-DDD2 with various IgG-AD2-modules to create a panel of IgG-IFNα for targeted delivery of IFNα to various malignancies.

The prototype, 20-2b-2b (a.k.a. 20-2b), which is a conjugate of IFNα2b-DDD2 and C_{H}3-AD2-IgG-hA20, comprises veltuzumab and four copies of IFNα2b (FIG. 4c). All of the IgG-IFNα made by DNL, including 20-2b-2b, retained the binding specificity and affinity of their targeting mAb, and also exhibited IFNα specific activity approaching that of recombinant IFNα2b (rIFNα2b). Cytokine fusion proteins made by traditional recombinant engineering or chemical conjugation often suffer extensive loss of biological activity. For example, where 20-2b-2b has similar activity to rIFNα2b, traditional recombinant anti-CD20-IFNα fusion proteins showed a 300-fold reduction in IFNα activity (Xuan et al., 2010, Blood 115:2864-2871). We believe that high-level activity of the DNL constructs is maintained due to the site-specific conjugation, which is spatially removed from the functional domain.

Compared to veltuzumab, 20-2b-2b showed enhanced ADCC, but lacked CDC (in vitro), consistent with other constructs comprising C_{H}3-AD2-IgG-hA20. It inhibited in-vitro proliferation of lymphoma cells and depleted them from whole human blood more potently than a combination of veltuzumab and IFNα. Comparative pharmacokinetic (Pk) analysis in mice demonstrated a longer circulating serum half-life for 20-2b-2b (23.4 h) compared to the commercial PEGylated IFNα drugs peginterferonalfa-2a (14.9 h) or peginterferonalfa-2b (9.3 h). Due to high specific activity, extended Pk and tumor targeting, 20-2b-2b demonstrated superior therapeutic efficacy compared to peginterferonalfa-2a, veltuzumab or non-targeting IgG-IFNα in three human lymphoma xenograft models, even though mouse immune cells respond poorly to human IFNα2b. Targeting IFNα with an anti-CD20 mAb made the immunocytokine more potent than either agent alone or in combination.

Based on encouraging preclinical results, 20-2b-2b, the prototype IgG-IFNα, is now under development for CD20-targeted immunotherapy of NHL. CD20 is a preferred target for this disease, because it is expressed at high levels on the cell surface of many B cell NHL, and its expression on normal cells is essentially limited to B cells. The potential benefits of therapy with 20-2b-2b are likely limited to NHL, hairy-cell leukemia, and possibly CLL patients. Using the combination of the IFNα2b-DDD2 module with C_{H}3-AD2-IgG-hL243, we have recently developed an IgG-IFNα named C2-2b-2b, which has tetrameric IFNα2b fused to an anti-HLA-DR mAb (hL243). HLA-DR is an attractive target because it is expressed on the cell surface of many hematopoietic malignancies (Stein et al., 2010, Blood 115:5180-5190). The broad range and high-level expression of HLA-DR makes C2-2b-2b attractive for use in therapy of diverse malignancies. In vitro, C2-2b-2b inhibited 20 cell lines, including B-cell lymphoma (Burkitt, mantle cell & follicular), leukemia (hairy cell, AML, ALL, and CLL), and myeloma. In most cases, this immunocytokine was more effective than CD20-targeted mAb-IFNα or a mixture comprising the parental mAb and IFNα. Responsiveness of each hematopoietic tumor cell line correlated with HLA-DR expression/density and sensitivity to IFNα and hL243. C2-2b-2b induced more potent and longer-lasting IFNα signaling compared to non-targeted IFNα. In vivo, C2-2b-2b demonstrated superior efficacy compared to non-targeting mAb-IFNα, peginterferonalfa-2a, or a combination of hL243 and IFNα using human lymphoma and myeloma xenografts.

The modular nature of DNL enabled the production of the first bispecific immunocytokine, 20-C2-2b, which comprises two copies of IFN-α2b and a stabilized F(ab), of hL243 (humanized anti-HLA-DR) site-specifically linked to veltuzumab (FIG. 4d) (Rossi et al., 2010, Cancer Res 70:7600-7609). This was achieved by combining C_{H}3-AD2-IgG-hA20 with two DDD-modules, IFNα2b-DDD2 and C_{H}1-DDD2-Fab-hL243. Due to the random association of either DDD-module with the two AD2 groups, two side-products, 20-C₂-C₂ and 20-2b-2b were formed, in addition to 20-C2-2b, which was purified from the mixture by a multi-step affinity chromatography process. The predicted biochemical and biological properties of 20-C2-2b were confirmed experimentally. It bound bispecifically to CD20+/HLA-DR+ cells. With two IFNα2b groups, 20-2b-2b exhibited half of the level of IFNα specific activity, compared to IgG-IFNα with four IFNα (e.g., 20-2b-2b). The bispecific IgG-IFNα potently inhibited lymphoma and myeloma cell lines, and in some cases, more effectively than 20-2b-2b or C2-2b-2b (Rossi et al., 2010, Cancer Res 70:7600-7609).

Several potential variations on the DNL method as described above are possible. In addition to the DDD sequence of human RIIα, other DDD sequences may be selected from human RIα, human RIβ, or human RIIβ. The DDD sequence of choice can be matched with a highly interactive AD sequence, which can be deduced from the literature (Burns-Hamuro et al., 2003, Proc Natl Acad Sci U.S.A, 100:4072-4077) or determined experimentally.

In addition to the use of disulfide linkages for preventing the dissociation of the assembled components, other methods for enhancing the overall stability of the tethered structure may be employed. For example, various cross-linking agents or methods that are commercially available or used in research may be selected. A potentially useful agent is glutaraldehyde, which has been widely used for probing the structures of non-covalently associated multimeric proteins by cross-linking the constituting subunits to form stable conjugates. Also of interest are two chemical methods involving oxidative cross-linking of protein subunits. One is a proximity labeling technique that employs either hexahistidine-tagged (Fancy et al., Chem Biol 3:551-559), or N-terminal glycine-glycine-histidine-tagged proteins (Brown et al., 1998, Biochemistry 37:4397-4406). These tags bind Ni²⁺ tightly and, when oxidized with a peracid, a Ni³⁺ species is produced to mediate a variety of oxidative reactions, including protein-protein cross-linking. Another technique, termed PICUP for photo-induced cross-linking of unmodified proteins, uses [Ru(II)(bipy)₃]²⁺, ammonium persulfate, and visible light to induce protein-protein cross-linking (Fancy & Kodadek, 1999, Proc Natl Acad Sci U.S.A, 96:6020-6024). However, these approaches may be less specific and efficient than the DDD2/AD2 coupling systems.

### Example 2

### Generation of C2/20-IgG, a bs2Fv-IgG Comprising Two Fᵥs of hL243 and one IgG of hA20

A plasmid DNA vector for expression of C2/20-IgG in murine myeloma cell culture is generated using the pdHL2 plasmid. The bs2Fv-IgG comprises two humanized Fᵥs of IMMU-114 and an IgG of hA20 for binding specifically to HLA-DR and human CD20, respectively. The construct, C2/20-IgG, is designed with the V_{H} and V_{L} domains of IMMU-114 fused to the amino terminal ends of the light chain and heavy chain of hA20, respectively. The expressed protein is comprised of two polypeptides, C2V_{H}-HL-20V_{K}-C_{K} (SEQ ID NO:120) and C2V_{K}-HL-20V_{H}-C_{H}1-C_{H}2-C_{H}3 (SEQ ID NO:121), with the tandem alternate variable domains separated by a hinge linker (HL), which is modified from the hinge region of murine IgG3 (FIG. 5). The amino acid sequences of the light chain and heavy chain polypeptides are shown in SEQ ID NO:120 and SEQ ID NO:121, respectively.

Molecular Engineering:

Two synthetic gene fragments are synthesized and cloned into staging vectors (SEQ ID NO:122 and SEQ ID NO:123). The synthetic genes were cloned into the hA20-pdHL2 expression vector in two steps. The hA20-pdHL2 vector fragment is prepared by digestion with XbaI and BstBI restriction endonucleases. The synthetic C2V_{H}-20V_{K} (SEQ ID NO:122) insert fragment is excised from its staging vector with XbaI and BstBI. Ligation of insert and vector fragments results in the generation of the intermediate vector hA20-(C2V_{H})-pdHL2. The hA20-(C2V_{H})-pdHL2 is digested with XhoI and HindIII restriction endonucleases to prepare the vector fragment. The synthetic C2V_{K}-20V_{H} (SEQ ID NO:123) insert fragment is excised from its staging vector with XhoI and HindIII. Ligation of insert and vector fragments results in the generation of the final expression vector C2/20-2Fv-IgG-pdHL2.

Protein Expression and Purification

The C2/20-2Fv-IgG-pdHL2 plasmid (30 μg) is linearized by digestion with Sal I and transfected into Sp/ESF (2.8×10⁶ cells) by electroporation (450 volts, 25 μF). The pdHL2 vector contains the gene for dihydrofolate reductase, thus allowing clonal selection as well as gene amplification with methotrexate (MTX). Following transfection, the cells are plated in 96-well plates and selected in media containing 0.2 μM MTX. Clones are screened for C2/20-IgG productivity by a sandwich ELISA using 96-well microtiter plates coated with anti-human Fc to capture the fusion protein, which is detected in independent assays with rat anti-idiotype MAbs to veltuzumab or Immu-114, followed by horseradish peroxidase-conjugated goat anti-rat IgG F(ab′)2. Wells giving the highest signal are expanded and ultimately used for production. C2/20-IgG is produced in roller bottles and purified from the culture supernatant fluid by Protein A affinity chromatography.

### Example 3

### Generation of 20/20/20-IgG, a Hexavalent Monospecific 4Fv-IgG

A plasmid DNA vector for expression of 20/20/20-IgG in murine myeloma cell culture is generated using the pdHL2 plasmid. The monospecific 4Fv-IgG comprises four Fᵥs of veltuzumab and an IgG of veltuzumab, for binding specifically to human CD20. The expressed protein, 20/20/20-IgG, comprises two polypeptides, 20V_{K}-SL-20V_{H}-HL-20V_{K}-C_{K} (SEQ ID NO:124) and 20V_{H}-SL-20V_{K}-HL-20V_{H}-C_{H}1-C_{H}2-C_{H}3 (SEQ ID NO:125), with the veltuzumab variable domains separated by a hinge linker (HL) and short linker (SL) as indicated (FIG. 6). The amino acid sequences of the light chain and heavy chain polypeptides are shown in SEQ ID NOS:124 and 125, respectively.

Molecular Engineering.

Two synthetic gene fragments are synthesized and cloned into staging vectors (SEQ ID NO:126 and SEQ ID NO:127). The synthetic genes are cloned into the hA20-pdHL2 expression vector in two steps. The hA20-pdHL2 vector fragment is prepared by digestion with XbaI and BstBI. The synthetic 20V_{K}-20V_{H}-20V_{K} (SEQ ID NO:126) insert fragment is excised from its staging vector with XbaI and BstBI. Ligation of insert and vector fragments results in the generation of the intermediate vector hA20-(20V_{K}-20V_{H}-20V_{K})-pdHL2.

The hA20-(20V_{K}-20V_{H}-20V_{K})-pdHL2 is digested with XhoI and HindIII to prepare the vector fragment. The synthetic 20V_{H}-20V_{K}-20V_{H} (SEQ ID NO:127) insert fragment is excised from its staging vector with XhoI and HindIII. Ligation of insert and vector fragments results in the generation of the final expression vector 20/20/20-4Fv-IgG-pdHL2.

The 20/20/20-4Fv-IgG-pdHL2expression vector is transfected, selected and screened (only using veltuzumab anti-idiotype), and the fusion protein, 20/20/20-IgG, is produced and purified using the same processes described in Example 1.

### Example 4

### Generation of 20/C2/20-IgG, a bs4Fv-IgG

A plasmid DNA vector for expression of 20/C2/20-IgG in murine myeloma cell culture is generated using the pdHL2 plasmid. 20/C2/20-IgG comprises two Fᵥs of veltuzumab, two Fᵥs of Immu-114 and an IgG of veltuzumab. The expressed protein, 20/C2/20-IgG, comprises two polypeptides, 20V_{K}-SL-C2V_{H}-HL-20V_{K}-C_{K} (SEQ ID NO:128) and 20V_{H}-SL-C2V_{K}-HL-20V_{H}-C_{H}1-C_{H}2-C_{H}3 (SEQ ID NO:129), with the variable domains separated by a hinge linker (HL) and short linker (SL) as indicated (FIG. 7). The amino acid sequences of the light chain and heavy chain polypeptides are shown in SEQ ID NO:128 and SEQ ID NO:129, respectively.

Molecular Engineering.

Two synthetic gene fragments are synthesized and cloned into staging vectors (SEQ ID NO:130 and SEQ ID NO:131). The synthetic genes are cloned into the hA20-pdHL2 expression vector in two steps. The hA20-pdHL2 vector fragment is prepared by digestion with XbaI and BstBI. The synthetic 20V_{K}-C2V_{H}-20V_{K} (SEQ ID NO:130) insert fragment is excised from its staging vector with XbaI and BstBI. Ligation of insert and vector fragments results in the generation of the intermediate vector hA20-(20V_{K}-C2V_{H}-20Vₓ)-pdHL2.

The hA20-(20V_{K}-C2V_{H}-20V_{K})-pdHL2 is digested with XhoI and HindIII to prepare the vector fragment. The synthetic 20V_{H}-C2V_{K}-20V_{H} (SEQ ID NO:131) insert fragment is excised from its staging vector with XhoI and HindIII Ligation of insert and vector fragments results in the generation of the final expression vector 20/C2/20-4Fv-IgG-pdHL2.

The 20/C2/20-4Fv-IgG-pdHL2 expression vector is transfected, selected and screened, and the fusion protein, 20/C2/20-IgG, is produced and purified using the same processes described in Example 1.

### Example 5

### Generation of 3/C2/20-IgG, a ts4Fv-IgG

A plasmid DNA vector for expression of 3/C2/20-IgG in murine myeloma cell culture is generated using the pdHL2 plasmid. 3/C2/20-IgG comprises two Fᵥs of hLR3, two Fᵥs of Immu-114 and an IgG of veltuzumab, which bind specifically to human CD3, HLA-DR and human CD20, respectively. The expressed protein, 3/C2/20-IgG, comprises two polypeptides, 3V_{K}-SL-C2V_{H}-HL-20V_{K}-C_{K} (SEQ ID NO:132) and 3V_{H}-SL-C2V_{K}-HL-20V_{H}-C_{H}1-C_{H}2-C_{H}3 (SEQ ID NO:133), with the variable domains separated by hinge linker (HL) and short linker (SL) as indicated (FIG. 8). The amino acid sequences of the light chain and heavy chain polypeptides are shown in SEQ ID NO:132 and SEQ ID NO:133, respectively.

Molecular Engineering—γTwo synthetic gene fragments are synthesized and cloned into staging vectors (SEQ ID NO:134 and SEQ ID NO:135). The synthetic genes are cloned into the hA20-pdHL2 expression vector in two steps. The hA20-pdHL2 vector fragment is prepared by digestion with XbaI and BstBI. The synthetic 3V_{K}-C2V_{H}-20V_{K} (SEQ ID NO:134) insert fragment is excised from its staging vector with XbaI and BstBI. Ligation of insert and vector fragments results in the generation of the intermediate vector hA20-(3V_{K}-C2V_{H}-20V_{K})-pdHL2.

The hA20-(3V_{K}-C2V_{H}-20V_{K})-pdHL2 is digested with XhoI and HindIII to prepare the vector fragment. The synthetic 3V_{H}-C2V_{K}-20V_{H} (SEQ ID NO:135) insert fragment is excised from its staging vector with XhoI and HindIII. Ligation of insert and vector fragments results in the generation of the final expression vector 3/C2/20-ts4Fv-IgG-pdHL2.

The 3/C2/20-ts4Fv-IgG-pdHL2 expression vector is transfected, selected and screened, and the fusion protein, 3/C2/20-IgG, is produced and purified using the same processes described in Example 1.

### Example 6

### Alternate ts4Fv-IgG Format #1

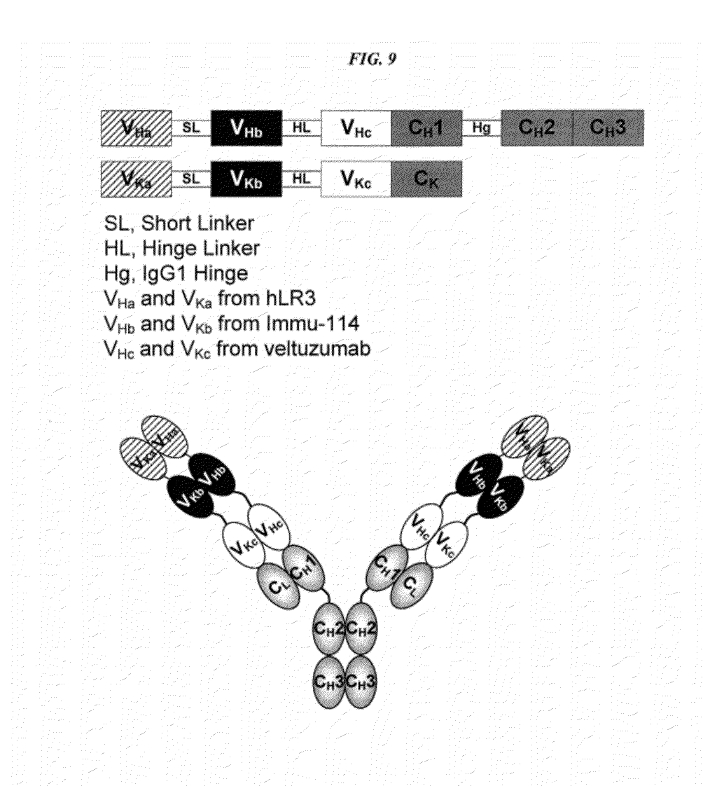

This alternate format for production of a ts4Fv-IgG (Example 5) is also applicable for the production of a monospecific 4Fv-IgG (Example 3), bs4Fv-IgG (Example 4) or bs2Fv-Fab (Example 9). Two synthetic genes are synthesized and ligated into the pdHL2 expression vector. The expressed protein, 3/C2/20-IgG-alt#1, comprises two polypeptides, 3V_{K}-SL-C2V_{K}-HL-20V_{K}-C_{K} (SEQ ID NO:136) and 3V_{H}-SL-C2V_{H}-HL-20V_{H}-C_{H}1-C_{H}2-C_{H}3 (SEQ ID NO:137) with the variable domains separated by a hinge linker (HL) and short linker (SL) as indicated (FIG. 9).

### Example 7

### Alternate ts4Fv-IgG Format #2

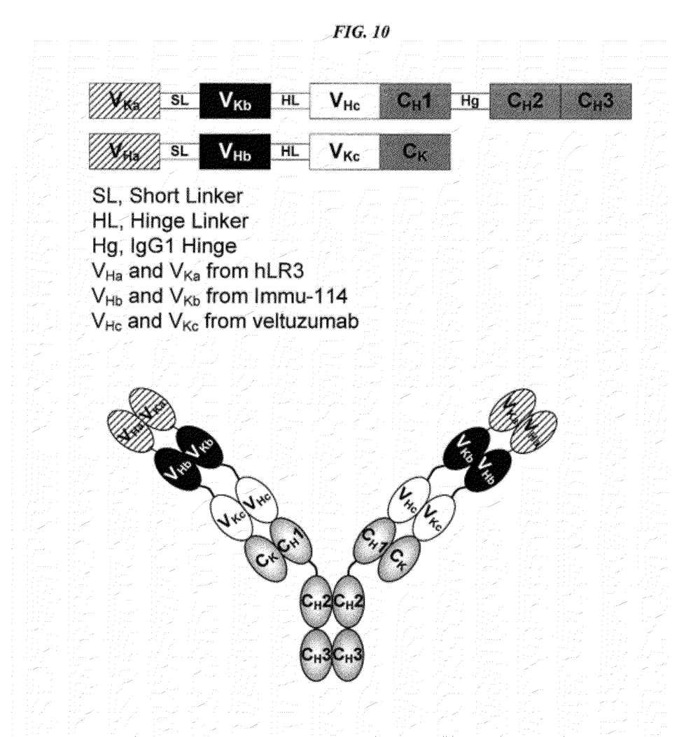

This alternate format for production of a ts4Fv-IgG (Example 5) is also applicable for the production of a monospecific 4Fv-IgG (Example 3), bs4Fv-IgG (Example 4) or bs2Fv-Fab (Example 9). Two synthetic genes are synthesized and ligated into the pdHL2 expression vector. The expressed protein, 3/C2/20-IgG-alt#2, comprises two polypeptides, 3V_{H}-SL-C2V_{H}-HL-20V_{K}-C_{K} (SEQ ID NO:138) and 3V_{K}-SL-C2V_{K}-HL-20V_{H}-C_{H}1-C_{H}2-C_{H}3 (SEQ ID NO:139) with the variable domains separated by a hinge linker (HL) and short linker (SL) as indicated (FIG. 10).

### Example 8

### Alternate ts4Fv-IgG Format #3

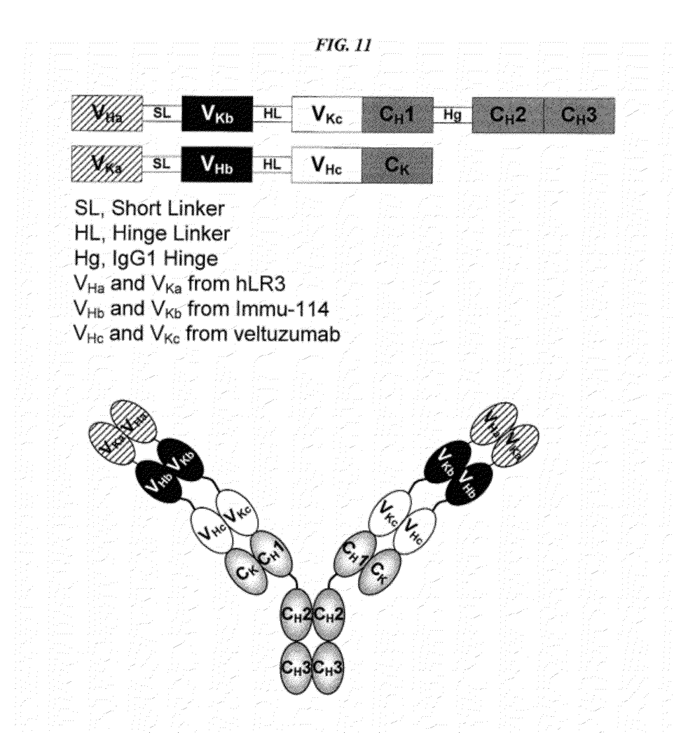

This alternate format for production of a ts4Fv-IgG (Example 5) is also applicable for the production of a monospecific 4Fv-IgG (Example 3), bs4Fv-IgG (Example 4) or bs2Fv-Fab (Example 9). Two synthetic genes are synthesized and ligated into the pdHL2 expression vector. The expressed protein, 3/C2/20-IgG-alt#3, comprises two polypeptides, 3V_{K}-SL-C2V_{H}-HL-20V_{H}-C_{K} (SEQ ID NO:140) and 3V_{H}-SL-C2V_{K}-HL-20V_{K}-C_{H}1-C_{H}2-C_{H}3 (SEQ ID NO:141) with the variable domains separated by a hinge linker (HL) and short linker (SL) as indicated (FIG. 11).

### Example 9

### Generation of 20/3/20-Fab, a bs2Fv-Fab

A plasmid DNA vector for expression of 20/3/20-Fab in murine myeloma cell culture is generated using the pdHL2 plasmid. 20/3/20-Fab comprises one Fᵥ of veltuzumab, one Fᵥ of hLR3 and an IgG of veltuzumab. The expressed protein, 20/3/20-Fab, comprises two polypeptides, 3V_{K}-SL-20V_{H}-HL-20V_{K}-C_{K} (SEQ ID NO:142) and 3V_{H}-SL-20V_{K}-HL-20V_{H}-C_{H}1 (SEQ ID NO:143), with the variable domains separated by hinge linker (HL) and short linker (SL) as indicated (FIG. 12). The amino acid sequences of the light chain and heavy chain polypeptides are shown in SEQ ID NO:142 and SEQ ID NO:143, respectively.

The 20/3/20-2Fv-Fab-pdHL2 expression vector is transfected and selected as described in Example 2. ELISA screening is accomplished by capturing with veltuzumab anti-idiotype MAb and detection with horseradish peroxidase-conjugated goat anti human Fab. The fusion protein, 20/3/20-Fab, is produced in roller bottle culture and purified from culture supernatant fluid using KappaSelect affinity chromatography.

### Example 10

### Generation of C2/20-IgG-AD2

To convert any of the multivalent IgG pdHL2 expression vectors, such as those described in Examples 1-8, into an expression vector for an IgG-AD2 module, an 861 bp BsrGI/VdeI restriction fragment is excised from the former and replaced with a 952 bp BsrGI/VdeI restriction fragment (SEQ ID NO:144) excised from any C_{H}3-AD2-IgG-pdHL2 vector. BsrGI cuts in the C_{H}3 domain and NdeI cuts downstream (3′) of the expression cassette.

C2/20-IgG-AD2 has the same light chain polypeptide (SEQ ID NO:120) as C2/20-IgG and a heavy chain polypeptide comprising C2V_{K}-HL-20V_{H}-C_{H}1-C_{H}2-C_{H}3-FL-AD2 (SEQ ID NO:145), where HL and FL are hinge linker and flexible linker peptides, respectively (FIG. 13). The FL and AD2 coding sequences are appended to the 3′ end of the coding sequence for the C_{H}3 domain in the C2/20-2Fv-IgG-pdHL2 vector by standard restriction digest/ligation molecular cloning methods. A 952 bp BsrGI/VdeI restriction fragment (SEQ ID NO:144) is excised from the C_{H}3-AD2-IgG-hA20-pdHL2 vector and ligated into the same restriction sites of C2/20-2Fv-IgG-pdHL2 (Example 1). The resulting expression vector C2/20-2Fv-IgG-AD2-pdHL2 is transfected, selected and screened, and the fusion protein, C2/20-IgG-AD2 (SEQ ID NO:145), is produced and purified using the same processes as described in Example 1.

### Example 11

### Generation of C2/20-Fab-DDD2

To convert any of the multivalent IgG pdHL2 expression vectors such as those described in Examples 1-8, into an expression vector for a Fab-DDD2 module, a 1498 bp AgeI/EagI restriction fragment is excised from the former and replaced with a 376 bp AgeI/EagI restriction fragment (SEQ ID NO:146) excised from any C_{H}1-DDD2-Fab-pdHL2 vector. AgeI cuts in the C_{H}1 domain and EagI cuts downstream (3′) of the expression cassette.

C2/20-Fab-DDD2 has the same light chain polypeptide (SEQ ID NO:120) as C2/20-Fab and a heavy chain polypeptide comprising C2V_{K}-HL-20V_{H}-C_{H}1-FL-DDD2 (SEQ ID NO:147), where HL and FL are hinge linker and flexible linker peptides, respectively (FIG. 14). The FL and DDD2 coding sequences are appended to the 3′ end of the coding sequence for the C_{H}1 domain, replacing the C_{H}2 and C_{H}3 domains, in the C2/20-2Fv-IgG-pdHL2 vector by standard restriction digest/ligation molecular cloning methods. A 367 bp AgeI/EagI restriction fragment (SEQ ID NO:146) is excised from the C_{H}1-DDD2-Fab-hMN-14-pdHL2 vector and ligated into the same restriction sites of C2/20-2Fv-IgG-pdHL2 (Example 2). The resulting expression vector C2/20-Fv-Fab-DDD2-pdHL2 is transfected, selected and screened, and the fusion protein, C2/20-Fab-DDD2, is produced and purified using the same processes described in Example 9.

### Example 12

### Preparation of Dock-and-Lock (DNL) Constructs

DDD and AD Fusion Proteins

The DNL technique can be used to make dimers, trimers, tetramers, hexamers, etc. comprising virtually any antibodies or fragments thereof or other effector moieties. For certain preferred embodiments, IgG antibodies, F(ab′)₂ antibody fragments, cytokines, xenoantigens and other effector moieties may be produced as fusion proteins containing either a dimerization and docking domain (DDD) or anchoring domain (AD) sequence. Although in preferred embodiments the DDD and AD moieties are produced as fusion proteins, the skilled artisan will realize that other methods of conjugation, such as chemical cross-linking, may be utilized within the scope of the claimed methods and compositions.

The DNL technique is not limiting and any protein or peptide of use may be produced as an AD or DDD fusion protein for incorporation into a DNL construct. Where chemical cross-linking is utilized, the AD and DDD conjugates are not limited to proteins or peptides and may comprise any molecule that may be cross-linked to an AD or DDD sequence using any cross-linking technique known in the art.

Independent transgenic cell lines may be developed for each DDD or AD fusion protein. Once produced, the modules can be purified if desired or maintained in the cell culture supernatant fluid. Following production, any DDD-fusion protein module can be combined with any AD-fusion protein module to generate a DNL construct. For different types of constructs, different AD or DDD sequences may be utilized.

Expression Vectors

The plasmid vector pdHL2 has been used to produce a number of antibodies and antibody-based constructs. See Gillies et al., J Immunol Methods (1989), 125:191-202; Losman et al., Cancer (Phila) (1997), 80:2660-6. The di-cistronic mammalian expression vector directs the synthesis of the heavy and light chains of IgG. The vector sequences are mostly identical for many different IgG-pdHL2 constructs, with the only differences existing in the variable domain (VH and VL) sequences. Using molecular biology tools known to those skilled in the art, these IgG expression vectors can be converted into Fab-DDD or Fab-AD expression vectors. To generate Fab-DDD expression vectors, the coding sequences for the hinge, CH2 and CH3 domains of the heavy chain are replaced with a sequence encoding the first 4 residues of the hinge, a 14 residue Gly-Ser linker and the first 44 residues of human RIIα (referred to as DDD1). To generate Fab-AD expression vectors, the sequences for the hinge, CH2 and CH3 domains of IgG are replaced with a sequence encoding the first 4 residues of the hinge, a 15 residue Gly-Ser linker and a 17 residue synthetic AD called AKAP-IS (referred to as AD1), which was generated using bioinformatics and peptide array technology and shown to bind RIIα dimers with a very high affinity (0.4 nM). See Alto, et al. Proc. Natl. Acad. Sci., U.S.A (2003), 100:4445-50.

Two shuttle vectors were designed to facilitate the conversion of IgG-pdHL2 vectors to either Fab-DDD1 or Fab-AD1 expression vectors, as described below.

Preparation of CH1

The CH1 domain was amplified by PCR using the pdHL2 plasmid vector as a template. The left PCR primer consisted of the upstream (5′) end of the CH1 domain and a SacII restriction endonuclease site, which is 5′ of the CH1 coding sequence. The right primer consisted of the sequence coding for the first 4 residues of the hinge followed by four glycines and a serine, with the final two codons (GS) comprising a Bam HI restriction site. The 410 bp PCR amplimer was cloned into the PGEMT® PCR cloning vector (PROMEGA®, Inc.) and clones were screened for inserts in the T7 (5′) orientation.

Construction of (G₄S)₂DDD1 ((G₄S)₂

A duplex oligonucleotide, designated (G₄S)₂DDD1 was synthesized by Sigma GENOSYS® (Haverhill, UK) to code for the amino acid sequence of DDD1 preceded by 11 residues of the linker peptide, with the first two codons comprising a BamHI restriction site. A stop codon and an EagI restriction site are appended to the 3′ end. The encoded polypeptide sequence is shown below.

(SEQ ID NO: 148)
GSGGGGSGGGGSHIQIPPGLTELLQGYTVEVLRQQPPDLVEFAVEYFTRL
REARA

Two oligonucleotides, designated RIIA1-44 top and RIIA1-44 bottom, that overlap by 30 base pairs on their 3′ ends, were synthesized (Sigma GENOSYS®) and combined to comprise the central 154 base pairs of the 174 bp DDD1 sequence. The oligonucleotides were annealed and subjected to a primer extension reaction with Taq polymerase. Following primer extension, the duplex was amplified by PCR. The amplimer was cloned into PGEMT® and screened for inserts in the T7 (5′) orientation.

Construction of (G₄S)₂-AD1

A duplex oligonucleotide, designated (G₄S)₂-AD1, was synthesized (Sigma GENOSYS®) to code for the amino acid sequence of AD1 preceded by 11 residues of the linker peptide with the first two codons comprising a BamHI restriction site. A stop codon and an EagI restriction site are appended to the 3′ end. The encoded polypeptide sequence is shown below.

(SEQ ID NO: 149)
GSGGGGSGGGGSQIEYLAKQIVDNAIQQA

Two complimentary overlapping oligonucleotides encoding the above peptide sequence, designated AKAP-IS Top and AKAP-IS Bottom, were synthesized and annealed. The duplex was amplified by PCR. The amplimer was cloned into the PGEMT® vector and screened for inserts in the T7 (5′) orientation.

Ligating DDD1 with CH1

A 190 bp fragment encoding the DDD1 sequence was excised from PGEMT® with BamHI and NotI restriction enzymes and then ligated into the same sites in CH1-PGEMT® to generate the shuttle vector CH1-DDD1-PGEMT®.

Ligating AD1 with CH1

A 110 bp fragment containing the AD1 sequence was excised from PGEMT® with BamHI and NotI and then ligated into the same sites in CH1-PGEMT® to generate the shuttle vector CH1-AD1-PGEMT®.

Cloning CH1-DDD1 or CH1-AD1 into pdHL2-Based Vectors

With this modular design either CH1-DDD1 or CH1-AD1 can be incorporated into any IgG construct in the pdHL2 vector. The entire heavy chain constant domain is replaced with one of the above constructs by removing the SacII/EagI restriction fragment (CH1-CH3) from pdHL2 and replacing it with the SacII/EagI fragment of CH1-DDD1 or CH1-AD1, which is excised from the respective pGemT shuttle vector.

Construction of h679-Fd-AD1-pdHL2

h679-Fd-AD1-pdHL2 is an expression vector for production of h679 Fab with AD1 coupled to the carboxyl terminal end of the CH1 domain of the Fd via a flexible Gly/Ser peptide spacer composed of 14 amino acid residues. A pdHL2-based vector containing the variable domains of h679 was converted to h679-Fd-AD1-pdHL2 by replacement of the SacII/EagI fragment with the CH1-AD1 fragment, which was excised from the CH1-AD1-SV3 shuttle vector with SacII and EagI.

Construction of C-DDD1-Fd-hMN-14-pdHL2

C-DDD1-Fd-hMN-14-pdHL2 is an expression vector for production of a stable dimer that comprises two copies of a fusion protein C-DDD1-Fab-hMN-14, in which DDD1 is linked to hMN-14 Fab at the carboxyl terminus of CH1 via a flexible peptide spacer. The plasmid vector hMN-14(I)-pdHL2, which has been used to produce hMN-14 IgG, was converted to C-DDD1-Fd-hMN-14-pdHL2 by digestion with SacII and EagI restriction endonucleases to remove the CH1-CH3 domains and insertion of the CH1-DDD1 fragment, which was excised from the CH1-DDD1-SV3 shuttle vector with SacII and EagI.

The same technique has been utilized to produce plasmids for Fab expression of a wide variety of known antibodies, such as hLL1, hLL2, hPAM4, hR1, hRS7, hMN-14, hMN-15, hA19, hA20 and many others. Generally, the antibody variable region coding sequences were present in a pdHL2 expression vector and the expression vector was converted for production of an AD- or DDD-fusion protein as described above.

Construction of C-DDD2-Fd-hMN-14-pdHL2

C-DDD2-Fd-hMN-14-pdHL2 is an expression vector for production of C-DDD2-Fab-hMN-14, which possesses a dimerization and docking domain sequence of DDD2 appended to the carboxyl terminus of the Fd of hMN-14 via a 14 amino acid residue Gly/Ser peptide linker. The fusion protein secreted is composed of two identical copies of hMN-14 Fab held together by non-covalent interaction of the DDD2 domains.

The expression vector was engineered as follows. Two overlapping, complimentary oligonucleotides, which comprise the coding sequence for part of the linker peptide (GGGGSGGGCG, SEQ ID NO:150) and residues 1-13 of DDD2, were made synthetically. The oligonucleotides were annealed and phosphorylated with T4 PNK, resulting in overhangs on the 5′ and 3′ ends that are compatible for ligation with DNA digested with the restriction endonucleases BamHI and PstI, respectively.

The duplex DNA was ligated with the shuttle vector CH1-DDD1-PGEMT®, which was prepared by digestion with BamHI and PstI, to generate the shuttle vector CH1-DDD2-PGEMT®. A 507 bp fragment was excised from CH1-DDD2-PGEMT® with SacII and EagI and ligated with the IgG expression vector hMN-14(I)-pdHL2, which was prepared by digestion with SacII and EagI. The final expression construct was designated C-DDD2-Fd-hMN-14-pdHL2. Similar techniques have been utilized to generated DDD2-fusion proteins of the Fab fragments of a number of different humanized antibodies.

Construction of h679-Fd-AD2-pdHL2

h679-Fd-AD2-pdHL2 is an expression vector for the production of h679-Fab-AD2, which possesses an anchoring domain sequence of AD2 appended to the carboxyl terminal end of the CH1 domain via a 14 amino acid residue Gly/Ser peptide linker. AD2 has one cysteine residue preceding and another one following the anchor domain sequence of AD1.

The expression vector was engineered as follows. Two overlapping, complimentary oligonucleotides which comprise the coding sequence for AD2 and part of the linker sequence, were made synthetically. The oligonucleotides were annealed and phosphorylated with T4 PNK, resulting in overhangs on the 5′ and 3′ ends that are compatible for ligation with DNA digested with the restriction endonucleases BamHI and SpeI, respectively.

The duplex DNA was ligated into the shuttle vector CH1-AD1-PGEMT®, which was prepared by digestion with BamHI and SpeI, to generate the shuttle vector CH1-AD2-PGEMT®. A 429 base pair fragment containing CH1 and AD2 coding sequences was excised from the shuttle vector with SacII and EagI restriction enzymes and ligated into h679-pdHL2 vector that prepared by digestion with those same enzymes. The final expression vector is h679-Fd-AD2-pdHL2.

Generation of TF2 Trimeric DNL Construct

A trimeric DNL construct designated TF2 was obtained by reacting C-DDD2-Fab-hMN-14 with h679-Fab-AD2. A pilot batch of TF2 was generated with >90% yield as follows. Protein L-purified C-DDD2-Fab-hMN-14 (200 mg) was mixed with h679-Fab-AD2 (60 mg) at a 1.4:1 molar ratio. The total protein concentration was 1.5 mg/ml in PBS containing 1 mM EDTA. Subsequent steps involved TCEP reduction, HIC chromatography, DMSO oxidation, and IMP 291 affinity chromatography. Before the addition of TCEP, SE-HPLC did not show any evidence of a₂b formation. Addition of 5 mM TCEP rapidly resulted in the formation of a₂b complex consistent with a 157 kDa protein expected for the binary structure. TF2 was purified to near homogeneity by IMP 291 affinity chromatography (not shown). IMP 291 is a synthetic peptide containing the HSG hapten to which the 679 Fab binds (Rossi et al., 2005, Clin Cancer Res 11:7122s-29s). SE-HPLC analysis of the IMP 291 unbound fraction demonstrated the removal of a₄, a₂ and free kappa chains from the product (not shown).

Non-reducing SDS-PAGE analysis demonstrated that the majority of TF2 exists as a large, covalent structure with a relative mobility near that of IgG (not shown). Reducing SDS-PAGE shows that any additional bands apparent in the non-reducing gel are product-related (not shown), as only bands representing the constituent polypeptides of TF2 were evident (not shown). However, the relative mobilities of each of the four polypeptides were too close to be resolved. MALDI-TOF mass spectrometry (not shown) revealed a single peak of 156,434 Da, which is within 99.5% of the calculated mass (157,319 Da) of TF2.

The functionality of TF2 was determined by BIACORE® assay. TF2, C-DDD1-hMN-14+h679-AD1 (used as a control sample of noncovalent a₂b complex), or C-DDD2-hMN-14+h679-AD2 (used as a control sample of unreduced a₂ and b components) were diluted to 1 μg/ml (total protein) and passed over a sensorchip immobilized with HSG. The response for TF2 was approximately two-fold that of the two control samples, indicating that only the h679-Fab-AD component in the control samples would bind to and remain on the sensorchip. Subsequent injections of W12 IgG, an anti-idiotype antibody for hMN-14, demonstrated that only TF2 had a DDD-Fab-hMN-14 component that was tightly associated with h679-Fab-AD as indicated by an additional signal response. The additional increase of response units resulting from the binding of W12 to TF2 immobilized on the sensorchip corresponded to two fully functional binding sites, each contributed by one subunit of C-DDD2-Fab-hMN-14. This was confirmed by the ability of TF2 to bind two Fab fragments of W12 (not shown).

### Example 13

### C_{H3}-AD2-IgG Expression Vectors

A plasmid shuttle vector was produced to facilitate the conversion of any IgG-pdHL2 vector into a C_{H3}-AD2-IgG-pdHL2 vector. The gene for the Fc (C_{H}, and C_{H3} domains) was amplified by PCR using the pdHL2 vector as a template and the following oligonucleotide primers:

Fc BglII Left
(SEQ ID NO: 151)
AGATCTGGCGCACCTGAACTCCTG
Fc Bam- EcoRI Right
(SEQ ID NO: 152)
GAATTCGGATCCTTTACCCGGAGACAGGGAGAG.

The amplimer was cloned in the pGemT PCR cloning vector (Promega). The Fc insert fragment was excised from pGemT with Xba I and Bam HI and ligated with AD2-pdHL2 vector that was prepared by digesting h679-Fab-AD2-pdHL2 (Rossi et al., Proc Natl Acad Sci USA 2006, 103:6841-6) with Xba I and Bam HI, to generate the shuttle vector Fc-AD2-pdHL2. To convert IgG-pdHL2 expression vectors to a C_{H3}-AD2-IgG-pdHL2 expression vectors, an 861 bp BsrG I/Nde I restriction fragment was excised from the former and replaced with a 952 bp BsrG I/Nde I restriction fragment excised from the Fc-AD2-pdHL2 vector. The following is a partial list of C_{H3}-AD2-IgG-pdHL2 expression vectors that have been generated and used for the production of recombinant humanized IgG-AD2 modules:

C_{H3}-AD2-IgG-hA20 (anti-CD20)

C_{H3}-AD2-IgG-hLL2 (anti-CD22)

C_{H3}-AD2-IgG-hL243 (anti-HLA-DR)

C_{H3}-AD2-IgG-hLL1 (anti-CD74)

C_{H3}-AD2-IgG-hR1 (anti-IGF-1R)

C_{H3}-AD2-IgG-h734 (anti-Indium-DTPA).

### Example 14

### Production of C_{H3}-AD2-IgG

Transfection and Selection of Stable C_{H3}-AD2-IgG Secreting Cell Lines

All cell lines were grown in Hybridoma SFM (Invitrogen, Carlsbad Calif.). C_{H3}-AD2-IgG-pdHL2 vectors (30 μg) were linearized by digestion with Sal I restriction endonuclease and transfected into Sp2/0-Ag14 (2.8×10⁶ cells) by electroporation (450 volts, 25 μF). The pdHL2 vector contains the gene for dihydrofolate reductase allowing clonal selection as well as gene amplification with methotrexate (MTX).

Following transfection, the cells were plated in 96-well plates and transgenic clones were selected in media containing 0.2 μM MTX. Clones were screened for C_{H3}-AD2-IgG productivity by a sandwich ELISA using 96-well microtitre plates coated with specific anti-idiotype MAbs. Conditioned media from the putative clones were transferred to the micro-plate wells and detection of the fusion protein was accomplished with horseradish peroxidase-conjugated goat anti-human IgG F(ab′)₂ (Jackson ImmunoResearch Laboratories, West Grove, Pa.). Wells giving the highest signal were expanded and ultimately used for production.

Production and Purification of C_{H3}-AD2-IgG Modules

For production of the fusion proteins, roller bottle cultures were seeded at 2×10⁵ cells/ml and incubated in a roller bottle incubator at 37° C. under 5% CO₂ until the cell viability dropped below 25% (−10 days). Culture broth was clarified by centrifugation, filtered, and concentrated up to 50-fold by ultrafiltration. For purification of C_{H3}-AD2-IgG modules, concentrated supernatant fluid was loaded onto a Protein-A (MAB Select) affinity column. The column was washed to baseline with PBS and the fusion proteins were eluted with 0.1 M Glycine, pH 2.5.

### Example 15

### Generation of 20/C2-IgG-AD2

A multivalent IgG-AD2 DNL module can alternatively be constructed similar to the method described in Example 2. This was accomplished for the multivalent IgG-AD2 DNL module 20/C2-IgG-AD2.

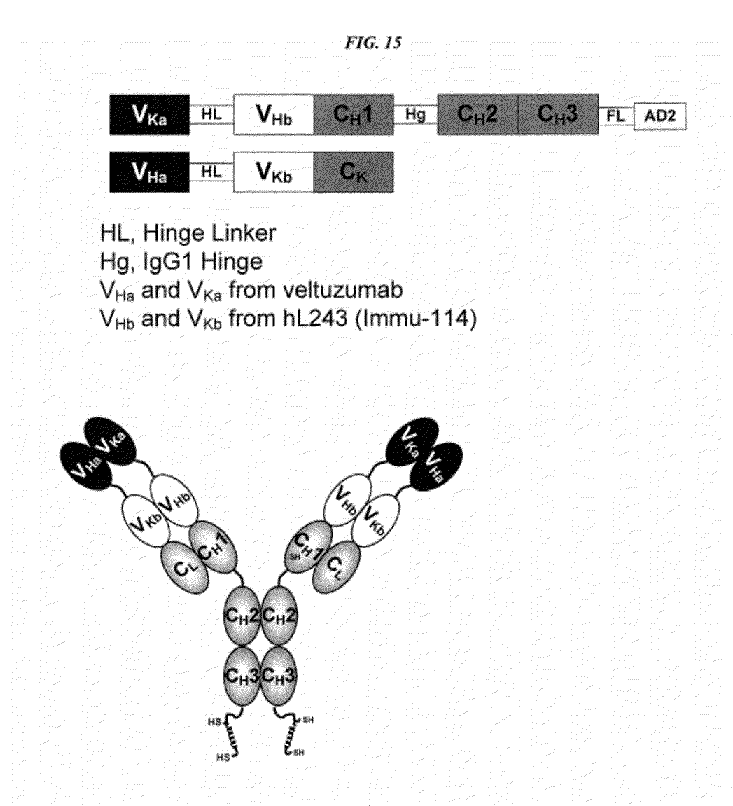

A plasmid DNA vector for expression of 20/C2-IgG-AD2 in murine myeloma cell culture was generated using the pdHL2 plasmid. The bs2Fv-IgG comprised two humanized Fᵥs of hA20 and an IgG of hL243 for binding specifically to human CD20 and HLA-DR, respectively. The construct, 20/C2-IgG-AD2, was designed with the V_{H} and V_{L} domains of hA20 fused to the amino terminal ends of the light chain and heavy chain of hL243, respectively. The expressed protein was comprised of two polypeptides, 20V_{H}-HL-C2V_{K}-C_{K} (SEQ ID NO:153) and 20V_{K}-HL-C2V_{H}-C_{H}1-C_{H}2-C_{H}3 (SEQ ID NO:154), with the tandem alternate variable domains separated by a hinge linker (HL), which was modified from the hinge region of murine IgG3 (FIG. 15). The amino acid sequences of the light chain and heavy chain polypeptides are shown in SEQ ID NO:153 and SEQ ID NO:154, respectively.

Molecular Engineering:

Two synthetic gene fragments were synthesized and cloned into staging vectors (SEQ ID NO:155 and SEQ ID NO:156). The synthetic genes were inserted into the hL243-IgG-AD2-pdHL2 expression vector in two steps. The C_{H}3-AD2-IgG-hL243-pdHL2 vector fragment was prepared by digestion with XbaI and NruI. The synthetic hA20V_{H}-hL243V_{K} insert fragment (SEQ ID NO:155) was excised from its staging vector with XbaI and NruI. Ligation of insert and vector fragments resulted in the generation of the intermediate vector C_{H}3-AD2-IgG-hL243-(hA20VH)-pdHL2.

The C_{H}3-AD2-IgG-hL243-(hA20VH)-pdHL2 vector was digested with XhoI and HindIII to prepare vector fragment. The synthetic hA20V_{K}-hL243V_{H} insert fragment SEQ ID NO:155) was isolated following digestion with XhoI and HindIII. Ligation of insert and vector fragments resulted in the generation of the final expression vector 20/C2-IgG-AD2-pdHL2.

Protein Expression and Purification

The 20/C2-IgG-AD2-pdHL2 plasmid (30 μg) was linearized by digestion with Sal I and transfected into Sp/ESF (2.8×10⁶ cells) by electroporation (450 volts, 25 μF). Following transfection, the cells were plated in 96-well plates and selected in media containing 0.2 μM MTX. Clones were screened for 20/C2-IgG-AD2 productivity by a sandwich ELISA using 96-well microtiter plates coated with anti-human Fc to capture the fusion protein, which was detected in independent assays with rat anti-idiotype MAbs to veltuzumab or hL243 (Immu-114), followed by horseradish peroxidase-conjugated goat anti-rat IgG F(ab′)2. Wells giving the highest signal were expanded and used for production. 20/C2-IgG-AD2 was produced in roller bottles and purified from the culture supernatant fluid by Protein A affinity chromatography.

Sequences

SEQ ID NO: 120.
Amino acid sequence for C2V_{H}-20V_{K} light chain.
Leader Peptide, C2V_{H}, Hinge Linker, 20V_{K}, C_{K}.
(SEQ ID NO: 120)
MGWSCIILFLVATATGVHSQVQLQQSGSELKKPGASVKVSCKASGFTFTN
YGMNWVKQAPGQGLKWMGWINTYTREPTYADDFKGRFAFSLDTSVSTAYL
QISSLKADDTAVYFCARDITAVVPTGFDYWGQGSLVTVSSEFPKPSTPPG
SSGGADIQLTQSPSSLSASVGDRVTMTCRASSSVSYIHWFQQKPGKAPKP
WIYATSNLASGVPVRFSGSGSGTDYTFTISSLQPEDIATYYCQQWTSNPP
TFGGGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
SEQ ID NO: 121.
Amino acid sequence for C2V_{H}-20V_{H} heavy chain.
Leader Peptide, C2V_{K}, Hinge Linker, 20V_{H},
C_{H}1-C_{H}3.
(SEQ ID NO: 121)
MGWSCIILFLVATATGVHSDIQLTQSPSSLSASVGDRVTITCRASENIYS
NLAWYRQKPGKAPKLLVFAASNLADGVPSRFSGSGSGTDYTFTISSLQPE
DIATYYCQHFWTTPWAFGGGTKLQIKREFPKPSTPPGSSGGAQVQLQQSG
AEVKKPGSSVKVSCKASGYTFTSYNMHWVKQAPGQGLEWIGAIYPGNGDT
SYNQKFKGKATLTADESTNTAYMELSSLRSEDTAFYYCARSTYYGGDWYF
DVWGQGTTVTVSSASTKGPSVFPLAPSSKSTSGGTAALGCLVKDYFPEPV
TVSWNSGALTSGVHTFPAVLQSSGLYSLSSVVTVPSSSLGTQTYICNVNH
KPSNTKVDKRVEPKSCDKTHTCPPCPAPELLGGPSVFLFPPKPKDTLMIS
RTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPS
REEMTKNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 122.
5′-3′ DNA sequence for C2V_{K}-20V_{H}. Cloning
restriction sites are underlined.
XhoI
(SEQ ID NO: 122)
CTCGAGCACACAGGACCTCACCATGGGATGGTCATGTATTATCCTCTTTC
TCGTGGCAACAGCAACAGGCGTCCATAGTGATATTCAGCTCACACAGTCC
CCTTCTTCTCTCAGCGCCAGCGTGGGCGACAGGGTCACTATCACCTGCAG
AGCATCAGAGAACATCTACAGCAACCTGGCCTGGTATCGACAGAAGCCTG
GCAAAGCTCCAAAGCTGCTCGTGTTCGCCGCTTCCAACCTCGCTGATGGA
GTCCCCAGCAGGTTCAGCGGAAGCGGATCCGGTACTGACTACACCTTCAC
CATCAGCTCCCTGCAGCCCGAGGATATTGCTACCTACTATTGCCAGCACT
TCTGGACCACACCTTGGGCATTTGGCGGAGGGACTAAACTGCAGATCAAG
AGGGAGTTCCCAAAACCCAGCACCCCACCTGGATCAAGCGGAGGAGCACA
GGTGCAGCTCCAGCAGAGTGGCGCTGAAGTCAAGAAACCCGGATCCTCTG
TGAAAGTCAGCTGTAAGGCCTCCGGCTACACCTTCACCAGCTATAACATG
CACTGGGTGAAGCAGGCACCTGGGCAGGGTCTGGAGTGGATCGGAGCCAT
CTACCCAGGCAACGGAGACACCTCCTATAATCAGAAGTTCAAAGGGAAGG
CAACCCTCACAGCCGATGAATCTACTAATACCGCTTACATGGAGCTGAGT
TCACTCCGGTCTGAAGATACAGCCTTTTACTATTGTGCTCGCAGTACTTA
CTACGGGGGGGATTGGTACTTCGACGTGTGGGGTCAGGGAACTACTGTCA
CTGTGTCCTCAGGTGAGTCCTTACAACCTCTCTCTTCTATTCAGCTTAAA
TAGATTTTACTGCATTTGTTGGGGGGGAAATGTGTGTATCTGAATTTCAG
GTCATGAAGGACTAGGGACACCTTGGGAGTCAGAAAGGGTCATTGGGGAT
CGCGGCCGCAAGCTT - Hind III
SEQ ID NO: 123.
5′-3′ DNA sequence for C2V_{H}-20V_{K}. Cloning
restriction sites are underlined.
Xba I
(SEQ ID NO: 123)
TCTAGACACAGGACCTCACCATGGGATGGAGTTGTATTATTCTCTTTCTG
GTCGCTACCGCTACCGGCGTGCATTCCCAGGTCCAGCTCCAGCAGTCCGG
TAGCGAACTCAAAAAGCCCGGCGCATCTGTGAAAGTCAGTTGCAAGGCCT
CAGGGTTCACCTTTACAAACTACGGTATGAATTGGGTGAAACAGGCTCCC
GGGCAGGGTCTGAAGTGGATGGGGTGGATCAACACTTACACCAGGGAGCC
TACATATGCTGACGATTTCAAAGGTAGATTCGCATTTTCCCTGGACACAA
GCGTGTCCACTGCATACCTGCAGATCAGCTCCCTCAAGGCCGACGATACT
GCTGTGTATTTCTGCGCTAGGGACATTACCGCAGTGGTCCCAACAGGCTT
TGATTATTGGGGCCAGGGATCACTGGTGACTGTCTCTAGTGAATTTCCAA
AACCCAGTACCCCACCTGGGTCTAGTGGTGGAGCAGACATTCAGCTGACA
CAGAGCCCCTCAAGCCTCTCTGCAAGTGTGGGCGACCGGGTCACCATGAC
ATGTCGCGCCTCCTCTAGTGTGTCCTACATTCACTGGTTTCAGCAGAAGC
CCGGTAAAGCCCCTAAGCCTTGGATCTACGCCACTTCGAA - BstBI
SEQ ID NO: 124.
Amino acid sequence for 20V_{K}-SL-20V_{H}-HL-20V_{K}-C_{K}.
Leader Peptide, 20V_{K}, short linker, 20V_{H}, Hinge
Linker, 20V_{K}, C_{K}
(SEQ ID NO: 124)
MGWSCIILFLVATATGVHSDIQLTQSPSSLSASVGDRVTMTCRASSSVSY
IHWFQQKPGKAPKPWIYATSNLASGVPVRFSGSGSGTDYTFTISSLQPED
IATYYCQQWTSNPPTFGGGTKLEIKRGGGGSQVQLQQSGAEVKKPGSSVK
VSCKASGYTFTSYNMHWVKQAPGQGLEWIGAIYPGNGDTSYNQKFKGKAT
LTADESTNTAYMELSSLRSEDTAFYYCARSTYYGGDWYFDVWGQGTTVTV
SSEFPKPSTPPGSSGGADIQLTQSPSSLSASVGDRVTMTCRASSSVSYIH
WFQQKPGKAPKPWIYATSNLASGVPVRFSGSGSGTDYTFTISSLQPEDIA
TYYCQQWTSNPPTFGGGTKLEIKRTVAAPSVFIFPPSDEQLKSGTASVVC
LLNNFYPREAKVQWKVDNALQSGNSQESVTEQDSKDSTYSLSSTLTLSKA
DYEKHKVYACEVTHQGLSSPVTKSFNRGEC
SEQ ID NO: 125.
Amino acid sequence for 20V_{H}-SL-20V_{K}-HL-20V_{H}-
C_{H}1-C_{H}2-C_{H}3. Leader Peptide, 20V_{H}, short linker,
20V_{K}, Hinge Linker, 20V_{H}, C_{H}1-C_{H}3
(SEQ ID NO: 125)
MGWSCIILFLVATATGVHSQVQLQQSGAEVKKPGSSVKVSCKASGYTFTS
YNMHWVKQAPGQGLEWIGAIYPGNGDTSYNQKFKGKATLTADESTNTAYM
ELSSLRSEDTAFYYCARSTYYGGDWYFDVWGQGTTVTVSSGGGGSDIQLT
QSPSSLSASVGDRVTMTCRASSSVSYIHWFQQKPGKAPKPWIYATSNLAS
GVPVRFSGSGSGTDYTFTISSLQPEDIATYYCQQWTSNPPTFGGGTKLEI
KREFPKPSTPPGSSGGAQVQLQQSGAEVKKPGSSVKVSCKASGYTFTSYN
MHWVKQAPGQGLEWIGAIYPGNGDTSYNQKFKGKATLTADESTNTAYMEL
SSLRSEDTAFYYCARSTYYGGDWYFDVWGQGTTVTVSSASTKGPSVFPLA
PSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMH
EALHNHYTQKSLSLSPGK
SEQ ID NO: 126.
5′-3′ DNA sequence for 20V_{K}-20V_{H}-20V_{K}
XbaI
(SEQ ID NO: 126)
TCTAGACACAGGACCTCACCATGGGATGGAGTTGTATTATTCTCTTTCTG
GTCGCTACCGCTACCGGCGTGCATTCCGACATTCAGCTGACACAGAGCCC
CTCAAGCCTCTCTGCAAGTGTGGGCGACCGGGTCACCATGACATGTCGCG
CCTCCTCTAGTGTGTCCTACATTCACTGGTTTCAGCAGAAGCCCGGTAAA
GCCCCTAAGCCTTGGATCTACGCCACTTCAAACCTGGCTTCTGGTGTCCC
TGTCCGATTCTCTGGCAGCGGATCTGGGACAGATTACACTTTCACCATCA
GCTCTCTTCAACCAGAAGACATTGCAACATATTATTGTCAGCAGTGGACT
AGTAACCCACCCACGTTCGGTGGAGGGACCAAGCTTCAGATCACCCGGGG
AGGTGGAGGCTCACAGGTGCAGCTCCAGCAGAGTGGCGCTGAAGTCAAGA
AACCCGGATCCTCTGTGAAAGTCAGCTGTAAGGCCTCCGGCTACACCTTC
ACCAGCTATAACATGCACTGGGTGAAGCAGGCACCTGGGCAGGGTCTGGA
GTGGATCGGAGCCATCTACCCAGGCAACGGAGACACCTCCTATAATCAGA
AGTTCAAAGGGAAGGCAACCCTCACAGCCGATGAATCTACTAATACCGCT
TACATGGAGCTGAGTTCACTCCGGTCTGAAGATACAGCCTTTTACTATTG
TGCTCGCAGTACTTACTACGGGGGGGATTGGTACTTCGACGTGTGGGGTC
AGGGAACTACTGTCACTGTGTCCTCAGAATTTCCAAAACCCAGTACCCCA
CCTGGGTCTAGTGGTGGAGCAGACATTCAGCTGACACAGAGCCCCTCAAG
CCTCTCTGCAAGTGTGGGCGACCGGGTCACCATGACATGTCGCGCCTCCT
CTAGTGTGTCCTACATTCACTGGTTTCAGCAGAAGCCCGGTAAAGCCCCT
AAGCCTTGGATCTACGCCACTTCGAA - BstBI
SEQ ID NO: 127.
5′-3′ DNA sequence for 20V_{H}-20V_{K}-20V_{H}
XhoI
(SEQ ID NO: 127)
CTCGAGCACACAGGACCTCACCATGGGATGGTCATGTATTATCCTCTTTC
TCGTGGCAACAGCAACAGGCGTCCATAGTCAGGTGCAGCTCCAGCAGAGT
GGCGCTGAAGTCAAGAAACCCGGATCCTCTGTGAAAGTCAGCTGTAAGGC
CTCCGGCTACACCTTCACCAGCTATAACATGCACTGGGTGAAGCAGGCAC
CTGGGCAGGGTCTGGAGTGGATCGGAGCCATCTACCCAGGCAACGGAGAC
ACCTCCTATAATCAGAAGTTCAAAGGGAAGGCAACCCTCACAGCCGATGA
ATCTACTAATACCGCTTACATGGAGCTGAGTTCACTCCGGTCTGAAGATA
CAGCCTTTTACTATTGTGCTCGCAGTACTTACTACGGGGGGGATTGGTAC
TTCGACGTGTGGGGTCAGGGAACTACTGTCACTGTGTCCTCAGGAGGTGG
AGGCTCAGACATTCAGCTGACACAGAGCCCCTCAAGCCTCTCTGCAAGTG
TGGGCGACCGGGTCACCATGACATGTCGCGCCTCCTCTAGTGTGTCCTAC
ATTCACTGGTTTCAGCAGAAGCCCGGTAAAGCCCCTAAGCCTTGGATCTA
CGCCACTTCAAACCTGGCTTCTGGTGTCCCTGTCCGATTCTCTGGCAGCG
GATCTGGGACAGATTACACTTTCACCATCAGCTCTCTTCAACCAGAAGAC
ATTGCAACATATTATTGTCAGCAGTGGACTAGTAACCCACCCACGTTCGG
TGGAGGGACCAAGCTTCAGATCACCCGGGAGTTCCCAAAACCCAGCACCC
CACCTGGATCAAGCGGAGGAGCACAGGTGCAGCTCCAGCAGAGTGGCGCT
GAAGTCAAGAAACCCGGATCCTCTGTGAAAGTCAGCTGTAAGGCCTCCGG
CTACACCTTCACCAGCTATAACATGCACTGGGTGAAGCAGGCACCTGGGC
AGGGTCTGGAGTGGATCGGAGCCATCTACCCAGGCAACGGAGACACCTCC
TATAATCAGAAGTTCAAAGGGAAGGCAACCCTCACAGCCGATGAATCTAC
TAATACCGCTTACATGGAGCTGAGTTCACTCCGGTCTGAAGATACAGCCT
TTTACTATTGTGCTCGCAGTACTTACTACGGGGGGGATTGGTACTTCGAC
GTGTGGGGTCAGGGAACTACTGTCACTGTGTCCTCAGGTGAGTCCTTACA
ACCTCTCTCTTCTATTCAGCTTAAATAGATTTTACTGCATTTGTTGGGGG
GGAAATGTGTGTATCTGAATTTCAGGTCATGAAGGACTAGGGACACCTTG
GGAGTCAGAAAGGGTCATTGGGGATCGCGGCCGCAAGCTT -
HindIII
SEQ ID NO: 128.
Amino acid sequence for 20V_{K}-SL-C2V_{H}-HL-20V_{K}-C_{K}.
Leader Peptide, 20V_{K}, short linker, C2V_{H}, Hinge
Linker, 20V_{K}, C_{K}.
(SEQ ID NO: 128)
MGWSCIILFLVATATGVHSDIQLTQSPSSLSASVGDRVTMTCRASSSVSY
IHWFQQKPGKAPKPWIYATSNLASGVPVRFSGSGSGTDYTFTISSLQPED
IATYYCQQWTSNPPTFGGGTKLEIKRGGGGSQVQLQQSGSELKKPGASVK
VSCKASGFTFTNYGMNWVKQAPGQGLKWMGWINTYTREPTYADDFKGRFA
FSLDTSVSTAYLQISSLKADDTAVYFCARDITAVVPTGFDYWGQGSLVTV
SSEFPKPSTPPGSSGGADIQLTQSPSSLSASVGDRVTMTCRASSSVSYIH
WFQQKPGKAPKPWIYATSNLASGVPVRFSGSGSGTDYTFTISSLQPEDIA
TYYCQQWTSNPPTFGGGTKLEIKRTVAAPSVFIFPPSDEQLKSGTASVVC
LLNNFYPREAKVQWKVDNALQSGNSQESVTEQDSKDSTYSLSSTLTLSKA
DYEKHKVYACEVTHQGLSSPVTKSFNRGEC
SEQ ID NO: 129.
Amino acid sequence for 20V_{H}-SL-C2V_{K}-HL-20V_{H}-
C_{H}1-C_{H}2-C_{H}3. Leader Peptide, 20V_{H}, short linker,
C2V_{K}, Hinge Linker, 20V_{H}, C_{H}1-C_{H}3
(SEQ ID NO: 129)
MGWSCIILFLVATATGVHSQVQLQQSGAEVKKPGSSVKVSCKASGYTFTS
YNMHWVKQAPGQGLEWIGAIYPGNGDTSYNQKFKGKATLTADESTNTAYM
ELSSLRSEDTAFYYCARSTYYGGDWYFDVWGQGTTVTVSSGGGGSDIQLT
QSPSSLSASVGDRVTITCRASENIYSNLAWYRQKPGKAPKLLVFAASNLA
DGVPSRFSGSGSGTDYTFTISSLQPEDIATYYCQHFWTTPWAFGGGTKLQ
IKREFPKPSTPPGSSGGAQVQLQQSGAEVKKPGSSVKVSCKASGYTFTSY
NMHWVKQAPGQGLEWIGAIYPGNGDTSYNQKFKGKATLTADESTNTAYME
LSSLRSEDTAFYYCARSTYYGGDWYFDVWGQGTTVTVSSASTKGPSVFPL
APSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPP
CPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVM
HEALHNHYTQKSLSLSPGK
SEQ ID NO: 130.
5′-3′ DNA sequence for 20V_{K}-C2V_{H}-20V_{K}
XbaI
(SEQ ID NO: 130)
TCTAGACACAGGACCTCACCATGGGATGGAGTTGTATTATTCTCTTTCTG
GTCGCTACCGCTACCGGCGTGCATTCCGACATTCAGCTGACACAGAGCCC
CTCAAGCCTCTCTGCAAGTGTGGGCGACCGGGTCACCATGACATGTCGCG
CCTCCTCTAGTGTGTCCTACATTCACTGGTTTCAGCAGAAGCCCGGTAAA
GCCCCTAAGCCTTGGATCTACGCCACTTCAAACCTGGCTTCTGGTGTCCC
TGTCCGATTCTCTGGCAGCGGATCTGGGACAGATTACACTTTCACCATCA
GCTCTCTTCAACCAGAAGACATTGCAACATATTATTGTCAGCAGTGGACT
AGTAACCCACCCACGTTCGGTGGAGGGACCAAGCTTCAGATCACCCGGGG
AGGTGGAGGCTCACAGGTCCAGCTCCAGCAGTCCGGTAGCGAACTCAAAA
AGCCCGGCGCATCTGTGAAAGTCAGTTGCAAGGCCTCAGGGTTCACCTTT
ACAAACTACGGTATGAATTGGGTGAAACAGGCTCCCGGGCAGGGTCTGAA
GTGGATGGGGTGGATCAACACTTACACCAGGGAGCCTACATATGCTGACG
ATTTCAAAGGTAGATTCGCATTTTCCCTGGACACAAGCGTGTCCACTGCA
TACCTGCAGATCAGCTCCCTCAAGGCCGACGATACTGCTGTGTATTTCTG
CGCTAGGGACATTACCGCAGTGGTCCCAACAGGCTTTGATTATTGGGGCC
AGGGATCACTGGTGACTGTCTCTAGTGAATTTCCAAAACCCAGTACCCCA
CCTGGGTCTAGTGGTGGAGCAGACATTCAGCTGACACAGAGCCCCTCAAG
CCTCTCTGCAAGTGTGGGCGACCGGGTCACCATGACATGTCGCGCCTCCT
CTAGTGTGTCCTACATTCACTGGTTTCAGCAGAAGCCCGGTAAAGCCCCT
AAGCCTTGGATCTACGCCACTTCGAA - BstBI
SEQ ID NO: 131.
5′-3′ DNA sequence for 20V_{H}-C2V_{K}-20V_{H}
XhoI
(SEQ ID NO: 131)
CTCGAGCACACAGGACCTCACCATGGGATGGTCATGTATTATCCTCTTTC
TCGTGGCAACAGCAACAGGCGTCCATAGTCAGGTGCAGCTCCAGCAGAGT
GGCGCTGAAGTCAAGAAACCCGGATCCTCTGTGAAAGTCAGCTGTAAGGC
CTCCGGCTACACCTTCACCAGCTATAACATGCACTGGGTGAAGCAGGCAC
CTGGGCAGGGTCTGGAGTGGATCGGAGCCATCTACCCAGGCAACGGAGAC
ACCTCCTATAATCAGAAGTTCAAAGGGAAGGCAACCCTCACAGCCGATGA
ATCTACTAATACCGCTTACATGGAGCTGAGTTCACTCCGGTCTGAAGATA
CAGCCTTTTACTATTGTGCTCGCAGTACTTACTACGGGGGGGATTGGTAC
TTCGACGTGTGGGGTCAGGGAACTACTGTCACTGTGTCCTCAGGAGGTGG
AGGCTCAGATATTCAGCTCACACAGTCCCCTTCTTCTCTCAGCGCCAGCG
TGGGCGACAGGGTCACTATCACCTGCAGAGCATCAGAGAACATCTACAGC
AACCTGGCCTGGTATCGACAGAAGCCTGGCAAAGCTCCAAAGCTGCTCGT
GTTCGCCGCTTCCAACCTCGCTGATGGAGTCCCCAGCAGGTTCAGCGGAA
GCGGATCCGGTACTGACTACACCTTCACCATCAGCTCCCTGCAGCCCGAG
GATATTGCTACCTACTATTGCCAGCACTTCTGGACCACACCTTGGGCATT
TGGCGGAGGGACTAAACTGCAGATCAAGAGGGAGTTCCCAAAACCCAGCA
CCCCACCTGGATCAAGCGGAGGAGCACAGGTGCAGCTCCAGCAGAGTGGC
GCTGAAGTCAAGAAACCCGGATCCTCTGTGAAAGTCAGCTGTAAGGCCTC
CGGCTACACCTTCACCAGCTATAACATGCACTGGGTGAAGCAGGCACCTG
GGCAGGGTCTGGAGTGGATCGGAGCCATCTACCCAGGCAACGGAGACACC
TCCTATAATCAGAAGTTCAAAGGGAAGGCAACCCTCACAGCCGATGAATC
TACTAATACCGCTTACATGGAGCTGAGTTCACTCCGGTCTGAAGATACAG
CCTTTTACTATTGTGCTCGCAGTACTTACTACGGGGGGGATTGGTACTTC
GACGTGTGGGGTCAGGGAACTACTGTCACTGTGTCCTCAGGTGAGTCCTT
ACAACCTCTCTCTTCTATTCAGCTTAAATAGATTTTACTGCATTTGTTGG
GGGGGAAATGTGTGTATCTGAATTTCAGGTCATGAAGGACTAGGGACACC
TTGGGAGTCAGAAAGGGTCATTGGGGATCGCGGCCGCAAGCTT -
HindIII
SEQ ID NO: 132.
Amino acid sequence for 3V_{K}-SL-C2V_{H}-HL-20V_{K}-C_{K}.
Leader Peptide, 3V_{K}, short linker, C2V_{H}, Hinge
Linker, 20V_{K}, C_{K}.
(SEQ ID NO: 132)
MGWSCIILFLVATATGVHSDIQLTQSPSSLSASVGDRVTMSCRASQSVSY
MNWYQQKPGKAPKLWIYDTSKVASGVPSRFSGSGSGTDYTFTISSLQPED
IATYYCQQNSSNPLTFGGGTKVQIKRGGGGSQVQLQQSGSELKKPGASVK
VSCKASGFTFTNYGMNWVKQAPGQGLKWMGWINTYTREPTYADDFKGRFA
FSLDTSVSTAYLQISSLKADDTAVYFCARDITAVVPTGFDYWGQGSLVTV
SSEFPKPSTPPGSSGGADIQLTQSPSSLSASVGDRVTMTCRASSSVSYIH
WFQQKPGKAPKPWIYATSNLASGVPVRFSGSGSGTDYTFTISSLQPEDIA
TYYCQQWTSNPPTFGGGTKLEIKRTVAAPSVFIFPPSDEQLKSGTASVVC
LLNNFYPREAKVQWKVDNALQSGNSQESVTEQDSKDSTYSLSSTLTLSKA
DYEKHKVYACEVTHQGLSSPVTKSFNRGEC
SEQ ID NO: 133.
Amino acid sequence for 3V_{H}-SL-C2V_{K}-HL-20V_{H}-
C_{H}1-C_{H}2-C_{H}3. Leader Peptide, 3V_{H}, short linker,
C2V_{K}, Hinge Linker, 20V_{H}, C_{H}1-C_{H}3
(SEQ ID NO: 133)
MGWSCIILFLVATATGVHSQVQLQQSGAEVKKPGSSVKVSCKASGYTFTR
YTMHWVRQAPGQGLEWIGYINPSRGYTNYADSVKGKATITADESTNTAYM
ELSSLRSEDTAFYYCARYYDDHYCLDYWGQGTTVTVSSGGGGSDIQLTQS
PSSLSASVGDRVTITCRASENIYSNLAWYRQKPGKAPKLLVFAASNLADG
VPSRFSGSGSGTDYTFTISSLQPEDIATYYCQHFWTTPWAFGGGTKLQIK
REFPKPSTPPGSSGGAQVQLQQSGAEVKKPGSSVKVSCKASGYTFTSYNM
HWVKQAPGQGLEWIGAIYPGNGDTSYNQKFKGKATLTADESTNTAYMELS
SLRSEDTAFYYCARSTYYGGDWYFDVWGQGTTVTVSSASTKGPSVFPLAP
SSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCP
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHE
ALHNHYTQKSLSLSPGK
SEQ ID NO: 134.
5′-3′ DNA sequence for 3V_{K}-C2V_{H}-20V_{K}.
XbaI
(SEQ ID NO: 134)
TCTAGACACAGGACCTCACCATGGGATGGAGTTGTATTATTCTCTTTCTG
GTCGCTACCGCTACCGGCGTGCATTCCGATATTCAGCTGACACAGTCTCC
ATCTTCCCTCAGTGCCTCCGTGGGCGATAGAGTCACAATGTCCTGCCGGG
CTTCACAGTCCGTGAGCTACATGAACTGGTATCAGCAGAAGCCCGGTAAA
GCCCCTAAGCTCTGGATCTACGACACCAGCAAAGTGGCTTCCGGAGTCCC
ATCTAGGTTCTCTGGCAGTGGATCAGGGACTGACTACACCTTTACAATCA
GCTCCCTGCAGCCCGAGGATATTGCCACCTACTATTGCCAGCAGAACAGC
AGCAACCCACTGACTTTTGGCGGAGGAACTAAGGTCCAGATTAAGAGGGG
AGGTGGAGGCTCACAGGTCCAGCTCCAGCAGTCCGGTAGCGAACTCAAAA
AGCCCGGCGCATCTGTGAAAGTCAGTTGCAAGGCCTCAGGGTTCACCTTT
ACAAACTACGGTATGAATTGGGTGAAACAGGCTCCCGGGCAGGGTCTGAA
GTGGATGGGGTGGATCAACACTTACACCAGGGAGCCTACATATGCTGACG
ATTTCAAAGGTAGATTCGCATTTTCCCTGGACACAAGCGTGTCCACTGCA
TACCTGCAGATCAGCTCCCTCAAGGCCGACGATACTGCTGTGTATTTCTG
CGCTAGGGACATTACCGCAGTGGTCCCAACAGGCTTTGATTATTGGGGCC
AGGGATCACTGGTGACTGTCTCTAGTGAATTTCCAAAACCCAGTACCCCA
CCTGGGTCTAGTGGTGGAGCAGACATTCAGCTGACACAGAGCCCCTCAAG
CCTCTCTGCAAGTGTGGGCGACCGGGTCACCATGACATGTCGCGCCTCCT
CTAGTGTGTCCTACATTCACTGGTTTCAGCAGAAGCCCGGTAAAGCCCCT
AAGCCTTGGATCTACGCCACTTCGAA - BstBI
SEQ ID NO: 135.
5′-3′ DNA sequence for 3V_{H}-C2V_{K}-20V_{H}.
(SEQ ID NO: 135)
CTCGAGCACACAGGACCTCACCATGGGATGGTCATGTATTATCCTCTTTC
TCGTGGCAACAGCAACAGGCGTCCATAGTCAGGTCCAGCTGCAGCAGTCC
GGGGCAGAGGTCAAGAAGCCAGGCAGCAGCGTCAAGGTGTCCTGTAAGGC
AAGCGGTTATACTTTTACAAGGTACACTATGCACTGGGTGAGACAGGCAC
CAGGACAGGGACTGGAGTGGATCGGGTATATTAACCCTTCTAGGGGTTAC
ACCAATTATGCTGACAGTGTCAAGGGAAAAGCCACCATCACAGCTGATGA
AAGCACTAACACCGCATACATGGAGCTGAGCTCCCTCCGGTCTGAAGACA
CAGCATTCTATTACTGCGCCCGCTATTACGACGACCATTACTGTCTGGAC
TACTGGGGGCAGGGGACTACGGTCACCGTCTCCTCAGGAGGTGGAGGCTC
AGATATTCAGCTCACACAGTCCCCTTCTTCTCTCAGCGCCAGCGTGGGCG
ACAGGGTCACTATCACCTGCAGAGCATCAGAGAACATCTACAGCAACCTG
GCCTGGTATCGACAGAAGCCTGGCAAAGCTCCAAAGCTGCTCGTGTTCGC
CGCTTCCAACCTCGCTGATGGAGTCCCCAGCAGGTTCAGCGGAAGCGGAT
CCGGTACTGACTACACCTTCACCATCAGCTCCCTGCAGCCCGAGGATATT
GCTACCTACTATTGCCAGCACTTCTGGACCACACCTTGGGCATTTGGCGG
AGGGACTAAACTGCAGATCAAGAGGGAGTTCCCAAAACCCAGCACCCCAC
CTGGATCAAGCGGAGGAGCACAGGTGCAGCTCCAGCAGAGTGGCGCTGAA
GTCAAGAAACCCGGATCCTCTGTGAAAGTCAGCTGTAAGGCCTCCGGCTA
CACCTTCACCAGCTATAACATGCACTGGGTGAAGCAGGCACCTGGGCAGG
GTCTGGAGTGGATCGGAGCCATCTACCCAGGCAACGGAGACACCTCCTAT
AATCAGAAGTTCAAAGGGAAGGCAACCCTCACAGCCGATGAATCTACTAA
TACCGCTTACATGGAGCTGAGTTCACTCCGGTCTGAAGATACAGCCTTTT
ACTATTGTGCTCGCAGTACTTACTACGGGGGGGATTGGTACTTCGACGTG
TGGGGTCAGGGAACTACTGTCACTGTGTCCTCAGGTGAGTCCTTACAACC
TCTCTCTTCTATTCAGCTTAAATAGATTTTACTGCATTTGTTGGGGGGGA
AATGTGTGTATCTGAATTTCAGGTCATGAAGGACTAGGGACACCTTGGGA
GTCAGAAAGGGTCATTGGGGATCGCGGCCGCAAGCTT
SEQ ID NO: 136.
Amino acid sequence for 3V_{K}-SL-C2V_{K}-HL-20V_{K}-C_{K}.
Leader Peptide, 3V_{K}, short linker, C2V_{K}, Hinge
Linker, 20V_{K}, C_{K}.
(SEQ ID NO: 136)
MGWSCIILFLVATATGVHSDIQLTQSPSSLSASVGDRVTMSCRASQSVSY
MNWYQQKPGKAPKLWIYDTSKVASGVPSRFSGSGSGTDYTFTISSLQPED
IATYYCQQNSSNPLTFGGGTKVQIKRGGGGSDIQLTQSPSSLSASVGDRV
TITCRASENIYSNLAWYRQKPGKAPKLLVFAASNLADGVPSRFSGSGSGT
DYTFTISSLQPEDIATYYCQHFWTTPWAFGGGTKLQIKREFPKPSTPPGS
SGGADIQLTQSPSSLSASVGDRVTMTCRASSSVSYIHWFQQKPGKAPKPW
IYATSNLASGVPVRFSGSGSGTDYTFTISSLQPEDIATYYCQQWTSNPPT
FGGGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEVT
HQGLSSPVTKSFNRGEC
SEQ ID NO: 137.
Amino acid sequence for 3V_{H}-SL-C2V_{H}-HL-20V_{H}-
C_{H}1-C_{H}2-C_{H}3. Leader Peptide, 3V_{H}, short linker,
C2V_{H}, Hinge Linker, 20V_{H}, C_{H}1-C_{H}3
(SEQ ID NO: 137)
MGWSCIILFLVATATGVHSQVQLQQSGAEVKKPGSSVKVSCKASGYTFTR
YTMHWVRQAPGQGLEWIGYINPSRGYTNYADSVKGKATITADESTNTAYM
ELSSLRSEDTAFYYCARYYDDHYCLDYWGQGTTVTVSSGGGGSQVQLQQS
GSELKKPGASVKVSCKASGFTFTNYGMNWVKQAPGQGLKWMGWINTYTRE
PTYADDFKGRFAFSLDTSVSTAYLQISSLKADDTAVYFCARDITAVVPTG
FDYWGQGSLVTVSSEFPKPSTPPGSSGGAQVQLQQSGAEVKKPGSSVKVS
CKASGYTFTSYNMHWVKQAPGQGLEWIGAIYPGNGDTSYNQKFKGKATLT
ADESTNTAYMELSSLRSEDTAFYYCARSTYYGGDWYFDVWGQGTTVTVSS
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 138.
Amino acid sequence for 3V_{H}-SL-C2V_{H}-HL-20V_{K}-C_{K}.
Leader Peptide, 3V_{H}, short linker, C2V_{H}, Hinge
Linker, 20V_{K}, C_{K}.
(SEQ ID NO: 138)
MGWSCIILFLVATATGVHSQVQLQQSGAEVKKPGSSVKVSCKASGYTFTR
YTMHWVRQAPGQGLEWIGYINPSRGYTNYADSVKGKATITADESTNTAYM
ELSSLRSEDTAFYYCARYYDDHYCLDYWGQGTTVTVSSGGGGSQVQLQQS
GSELKKPGASVKVSCKASGFTFTNYGMNWVKQAPGQGLKWMGWINTYTRE
PTYADDFKGRFAFSLDTSVSTAYLQISSLKADDTAVYFCARDITAVVPTG
FDYWGQGSLVTVSSEFPKPSTPPGSSGGADIQLTQSPSSLSASVGDRVTM
TCRASSSVSYIHWFQQKPGKAPKPWIYATSNLASGVPVRFSGSGSGTDYT
FTISSLQPEDIATYYCQQWTSNPPTFGGGTKLEIKRTVAAPSVFIFPPSD
EQLKSGTASVVCLLNNFYPREAKVQWKVDNALQSGNSQESVTEQDSKDST
YSLSSTLTLSKADYEKHKVYACEVTHQGLSSPVTKSFNRGEC
SEQ ID NO: 139.
Amino acid sequence for 3V_{K}-SL-C2V_{K}-HL-20V_{H}-
C_{H}1-C_{H}2-C_{H}3. Leader Peptide, 3V_{K}, short linker,
C2V_{K}, Hinge Linker, 20V_{H}, C_{H}1-C_{H}3
(SEQ ID NO: 139)
MGWSCIILFLVATATGVHSDIQLTQSPSSLSASVGDRYTMSCRASQSVSY
MNWYQQKPGKAPKLWIYDTSKVASGVPSRFSGSGSGTDYTFTISSLQPED
IATYYCQQNSSNPLTFGGGTKVQIKRGGGGSDIQLTQSPSSLSASVGDRV
TITCRASENIYSNLAWYRQKPGKAPKLLVFAASNLADGVPSRFSGSGSGT
DYTFTISSLQPEDIATYYCQHFWTTPWAFGGGTKLQIKREFPKPSTPPGS
SGGAQVQLQQSGAEVKKPGSSVKVSCKASGYTFTSYNMHWVKQAPGQGLE
WIGAIYPGNGDTSYNQKFKGKATLTADESTNTAYMELSSLRSEDTAFYYC
ARSTYYGGDWYFDVWGQGTTVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYK
TTPPVLDSDGSFELYSKLTVDKSRWQQGNVFSCSVMHEALHNHYTQKSLS
LSPGK
SEQ ID NO: 140.
Amino acid sequence for 3V_{K}-SL-C2V_{H}-HL-20V_{H}-C_{K}.
Leader Peptide, 3V_{K}, short linker, C2V_{H}, Hinge
Linker, 20V_{H}, C_{K}.
(SEQ ID NO: 140)
MGWSCIILFLVATATGVHSDIQLTQSPSSLSASVGDRVTMSCRASQSVSY
MNWYQQKPGKAPKLWIYDTSKVASGVPSRFSGSGSGTDYTFTISSLQPED
IATYYCQQNSSNPLTFGGGTKVQIKRGGGGSQVQLQQSGSELKKPGASVK
VSCKASGFTFTNYGMNWVKQAPGQGLKWMGWINTYTREPTYADDFKGRFA
FSLDTSVSTAYLQISSLKADDTAVYFCARDITAVVPTGFDYWGQGSLVTV
SSEFPKPSTPPGSSGGAQVQLQQSGAEVKKPGSSVKVSCKASGYTFTSYN
MHWVKQAPGQGLEWIGAIYPGNGDTSYNQKFKGKATLTADESTNTAYMEL
SSLRSEDTAFYYCARSTYYGGDWYEDVWGQGTTVTVSSTVAAPSVFIFPP
SDEQLKSGTASVVCLLNNFYPREAKVQWKVDNALQSGNSQESVTEQDSKD
STYSLSSTLTLSKADYEKHKVYACEVTHQGLSSPVTKSFNRGEC
SEQ ID NO: 141.
Amino acid sequence for 3V_{H}-SL-C2V_{K}-HL-20V_{K}-
C_{H}1-C_{H}2-C_{H}3. Leader Peptide, 3V_{H}, short linker,
C2V_{K}, Hinge Linker, 20V_{K}, C_{H}1-C_{H}3
(SEQ ID NO: 141)
MGWSCIILFLVATATGVHSQVQLQQSGAEVKKPGSSVKVSCKASGYTFTR
YTMHWVRQAPGQGLEWIGYINPSRGYTNYADSVKGKATITADESTNTAYM
ELSSLRSEDTAFYYCARYYDDHYCLDYWGQGTTVTVSSGGGGSDIQLTQS
PSSLSASVGDRVTITCRASENIYSNLAWYRQKPGKAPKLLVFAASNLADG
VPSRFSGSGSGTDYTFTISSLQPEDIATYYCQHFWTTPWAFGGGTKLQIK
REFPKPSTPPGSSGGADIQLTQSPSSLSASVGDRVTMTCRASSSVSYIHW
FQQKPGKAPKPWIYATSNLASGVPVRFSGSGSGTDYTFTISSLQPEDIAT
YYCQQWTSNPPTFGGGTKLEIKRASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK
SEQ ID NO: 142.
Amino acid sequence for 3V_{K}-SL-20V_{H}-HL-20V_{K}-C_{K}.
Leader Peptide, 3V_{K}, short linker, 20V_{H}, Hinge
Linker, 20V_{K}, C_{K}.
(SEQ ID NO: 142)
MGWSCIILFLVATATGVHSDIQLTQSPSSLSASVGDRVTMSCRASQSVSY
MNWYQQKPGKAPKLWIYDTSKVASGVPSRFSGSGSGTDYTFTISSLQPED
IATYYCQQNSSNPLTFGGGTKVQIKRGGGGSQVQLQQSGAEVKKPGSSVK
VSCKASGYTFTSYNMHWVKQAPGQGLEWIGAIYPGNGDTSYNQKFKGKAT
LTADESTNTAYMELSSLRSEDTAFYYCARSTYYGGDWYFDVWGQGTTVTV
SSEFPKPSTPPGSSGGADIQLTQSPSSLSASVGDRVTMTCRASSSVSYIH
WFQQKPGKAPKPWIYATSNLASGVPVRFSGSGSGTDYTFTISSLQPEDIA
TYYCQQWTSNPPTFGGGTKLEIKRTVAAPSVFIFPPSDEQLKSGTASVVC
LLNNFYPREAKVQWKVDNALQSGNSQESVTEQDSKDSTYSLSSTLTLSKA
DYEKHKVYACEVTHQGLSSPVTKSFNRGEC
SEQ ID NO: 143.
Amino acid sequence for 3V_{H}-SL-C2V_{K}-HL-20V_{H}-C_{H}1.
Leader Peptide, C2V_{H}, short linker, 20V_{K}, Hinge
Linker, 20V_{H}, C_{H}1.
(SEQ ID NO: 143)
MGWSCIILFLVATATGVHSQVQLQQSGAEVKKPGSSVKVSCKASGYTFTR
YTMHWVRQAPGQGLEWIGYINPSRGYTNYADSVKGKATITADESTNTAYM
ELSSLRSEDTAFYYCARYYDDHYCLDYWGQGTTVTVSSGGGGSDIQLTQS
PSSLSASVGDRVTMTCRASSSVSYIHWFQQKPGKAPKPWIYATSNLASGV
PVRFSGSGSGTDYTFTISSLQPEDIATYYCQQWTSNPPTFGGGTKLEIKR
EFPKPSTPPGSSGGAQVQLQQSGAEVKKPGSSVKVSCKASGYTFTSYNMH
WVKQAPGQGLEWIGAIYPGNGDTSYNQKFKGKATLTADESTNTAYMELSS
LRSEDTAFYYCARSTYYGGDWYFDVWGQGTTVTVSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKKPKSC
Sequence 144.
5′-3′ DNA sequence for AD2 conversion
(SEQ ID NO: 144)
TGTACACCCTGCCCCCATCCCGGGAGGAGATGACCAAGAACCAGGTCAGC
CTGACCTGCCTGGTCAAAGGCTTCTATCCCAGCGACATCGCCGTGGAGTG
GGAGAGCAATGGGCAGCCGGAGAACAACTACAAGACCACGCCTCCCGTGC
TGGACTCCGACGGCTCCTTCTTCCTCTATAGCAAGCTCACCGTGGACAAG
AGCAGGTGGCAGCAGGGGAACGTCTTCTCATGCTCCGTGATGCATGAGGC
TCTGCACAACCACTACACGCAGAAGAGCCTCTCCCTGTCTCCGGGTAAAG
GATCCGGAGGTGGCGGGTCTGGCGGATGTGGCCAGATCGAGTACCTGGCC
AAGCAGATCGTGGACAACGCCATCCAGCAGGCCGGCTGCTGAACTAGTGC
GGCCGGCAAGCCCCCGCTCCCCGGGCTCTCGCGGTCGCACGAGGATGCTT
GGCACGTACCCCGTCTACATACTTCCCAGGCACCCAGCATGGAAATAAAG
CACCCACCACTGCCCTGGGCCCCTGCGAGACTGTGATGGTTCTTTCCACG
GGTCAGGCCGAGTCTGAGGCCTGAGTGGCATGAGGGAGGCAGAGCGGGTC
CCACTGTCCCCACACTGGCCCAGGCTGTGCAGGTGTGCCTGGGCCGCCTA
GGGTGGGGCTCAGCCAGGGGCTGCCCTCGGCAGGGTGGGGGATTTGCCAG
CGTGGCCCTCCCTCCAGCAGCAGCTGCCTCGCGCGTTTCGGTGATGACGG
TGAAAACCTCTGACACATGCAGCTCCCGGAGACGGTCACAGCTTGTCTGT
AAGCGGATGCCGGGAGCAGACAAGCCCGTCAGGGCGCGTCAGCGGGTGTT
GGCGGGTGTCGGGGCGCAGCCATGACCCAGTCACGTAGCGATAGCGGAGT
GTATACTGGCTTAACTATGCGGCATCAGAGCAGATTGTACTGAGAGTGCA
CCATATG
Sequence 145.
Amino acid sequence for C2V_{H}-20V_{H} heavy
chain-AD2. Leader Peptide, C2V_{K}, Hinge
Linker, 20V_{H}, C_{H}1-C_{H}3, Flexible linker, AD2.
(SEQ ID NO: 145)
MGWSCIILFLVATATGVHSDIQLTQSPSSLSASVGDRVTITCRASENIYS
NLAWYRQKPGKAPKLLVFAASNLADGVPSRFSGSGSGTDYTFTISSLQPE
DIATYYCQHFWTTPWAFGGGTKLQIKREFPKPSTPPGSSGGAQVQLQQSG
AEVKKPGSSVKVSCKASGYTFTSYNMHWVKQAPGQGLEWIGAIYPGNGDT
SYNQKFKGKATLTADESTNTAYMELSSLRSEDTAFYYCARSTYYGGDWYF
DVWGQGTTVTVSSASTKGPSVFPLAPSSKSTSGGTAALGCLVKDYFPEPV
TVSWNSGALTSGVHTFPAVLQSSGLYSLSSVVTVPSSSLGTQTYICNVNH
KPSNTKVDKRVEPKSCDKTHTCPPCPAPELLGGPSVFLFPPKPKDTLMIS
RTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPS
REEMTKNQVSLTCLVKGFYPSDIAVEWESNGQPENNYKTTPPVLDSDGSF
FLYSKLTVDKSRWQQGNVFSCSVMHEALHNHYTQKSLSLSPGKGSGGGGS
GGCGQIEYLAKQIVDNAIQQAGC
SEQ ID NO: 146.
5′-3′ DNA sequence for Fab-DDD2 conversion
(SEQ ID NO: 146)
ACCGGTGACGGTGTCGTGGAACTCAGGCGCCCTGACCAGCGGCGTGCACA
CCTTCCCGGCTGTCCTACAGTCCTCAGGACTCTACTCCCTCAGCAGCGTG
GTGACCGTGCCCTCCAGCAGCTTGGGCACCCAGACCTACATCTGCAACGT
GAATCACAAGCCCAGCAACACCAAGGTGGACAAGAAACCTAAGAGCTGCG
GCGGCGGAGGATCCGGAGGTGGCGGGTCTGGCGGAGGTTGCGGCCACATC
CAGATCCCGCCGGGGCTCACGGAGCTGCTGCAGGGCTACACGGTGGAGGT
GCTGCGACAGCAGCCGCCTGACCTCGTCGAATTCGCAGTGGAGTACTTCA
CCCGCCTGAGAGAAGCTCGCGCTTGACGGCCG
SEQ ID NO: 147.
Amino acid sequence for C2V_{K}-HL-20V_{H}-C_{H}1-
FL-DDD2. Leader Peptide, C2V_{K}, Hinge Linker,
20V_{H}, C_{H}1-C_{H}3, Flexible linker, DDD2.
(SEQ ID NO: 147)
MGWSCIILFLVATATGVHSDIQLTQSPSSLSASVGDRVTITCRASENIYS
NLAWYRQKPGKAPKLLVFAASNLADGVPSRFSGSGSGTDYTFTISSLQPE
DIATYYCQHFWTTPWAFGGGTKLQIKREFPKPSTPPGSSGGAQVQLQQSG
AEVKKPGSSVKVSCKASGYTFTSYNMHWVKQAPGQGLEWIGAIYPGNGDT
SYNQKFKGKATLTADESTNTAYMELSSLRSEDTAFYYCARSTYYGGDWYF
DVWGQGTTVTVSSASTKGPSVFPLAPSSKSTSGGTAALGCLVKDYFPEPV
TVSWNSGALTSGVHTFPAVLQSSGLYSLSSVVTVPSSSLGTQTYICNVNH
KPSNTKVDKKPKSCGGGGSGGGGSGGGCGHIQIPPGLTELLQGYTVEVLR
QQPPDLVEFAVEYFTRLREARA
SEQ ID NO: 153
Amino acid sequence for hA20V_{H}-hL243Vₖ light
chain. Leader Peptide, hA20VH, Hinge Linker
hL243Vk, CK.
(SEQ ID NO: 153)
MGWSCIILFLVATATGVHSQVQLQQSGAEVKKPGSSVKVSCKASGYTFTS
YNMHWVKQAPGQGLEWIGAIYPGNGDTSYNQKFKGKATLTADESTNTAYM
ELSSLRSEDTAFYYCARSTYYGGDWYFDVWGQGTTVTVSSEFPKPSTPPG
SSGGADIQLTQSPSSLSASVGDRVTITCRASENIYSNLAWYRQKPGKAPK
LLVFAASNLADGVPSRFSGSGSGTDYTFTISSLQPEDIATYYCQHFWTTP
WAFGGGTKLQIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
SEQ ID NO: 154.
Amino acid sequence for 20V_{K}-C2V_{H} heavy
chain-AD2. Leader Peptide, 20V_{K}, Hinge
Linker, C2V_{H}, C_{H}1-C_{H}3, Flexible linker, AD2.
(SEQ ID NO: 154)
MGWSCIILFLVATATGVHSDIQLTQSPSSLSASVGDRVTMTCRASSSVSY
IHWFQQKPGKAPKPWIYATSNLASGVPVRFSGSGSGTDYTFTISSLQPED
IATYYCQQWTSNPPTFGGGTKLEIKREFPKPSTPPGSSGGAQVQLQQSGS
ELKKPGASVKVSCKASGFTFTNYGMNWVKQAPGQGLKWMGWINTYTREPT
YADDFKGRFAFSLDTSVSTAYLQISSLKADDTAVYFCARDITAVVPTGFD
YWGQGSLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHK
PSNTKVDKRVEPKSCDKTHTCPPCPAPELLGGPSVFLFPPKPKDTLMISR
TPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFF
LYSKLTVDKSRWQQGNVFSCSVMHEALHNHYTQKSLSLSPGKGSGGGGSG
GCGQIEYLAKQIVDNAIQQAGC
SEQ ID NO: 155.
5′-3′ DNA sequence for 20V_{H}-C2V_{K}.
(SEQ ID NO: 155)
TCTAGACACAGGACCTCACCATGGGATGGAGTTGTATTATTCTCTTTCTG
GTCGCTACCGCTACCGGCGTGCATTCCCAGGTGCAGCTCCAGCAGAGTGG
CGCTGAAGTCAAGAAACCCGGATCCTCTGTGAAAGTCAGCTGTAAGGCCT
CCGGCTACACCTTCACCAGCTATAACATGCACTGGGTGAAGCAGGCACCT
GGGCAGGGTCTGGAGTGGATCGGAGCCATCTACCCAGGCAACGGAGACAC
CTCCTATAATCAGAAGTTCAAAGGGAAGGCAACCCTCACAGCCGATGAAT
CTACTAATACCGCTTACATGGAGCTGAGTTCACTCCGGTCTGAAGATACA
GCCTTTTACTATTGTGCTCGCAGTACTTACTACGGGGGGGATTGGTACTT
CGACGTGTGGGGTCAGGGAACTACTGTCACTGTGTCCTCAGAATTTCCAA
AACCCAGTACCCCACCTGGGTCTAGTGGTGGAGCAGACATCCAGCTGACC
CAGTCTCCATCATCTCTGAGCGCATCTGTTGGAGATAGGGTCACTATCAC
TTGTCGAGCAAGTGAGAATATTTACAGTAATTTAGCATGGTATCGTCAGA
AACCAGGGAAAGCACCTAAACTGCTGGTCTTTGCTGCATCAAACTTAGCA
GATGGTGTGCCTTCGCGA
SEQ ID NO: 156.
5′-3′ DNA sequence for 20Vₖ-C2V_{H}
(SEQ ID NO: 156)
CTCGAGCACACAGGACCTCACCATGGGATGGTCATGTATTATCCTCTTTC
TCGTGGCAACAGCAACAGGCGTCCATAGTGACATTCAGCTGACACAGAGC
CCCTCAAGCCTCTCTGCAAGTGTGGGCGACCGGGTCACCATGACATGTCG
CGCCTCCTCTAGTGTGTCCTACATTCACTGGTTTCAGCAGAAGCCCGGTA
AAGCCCCTAAGCCTTGGATCTACGCCACTTCAAACCTGGCTTCTGGTGTC
CCTGTCCGATTCTCTGGCAGCGGATCTGGGACAGATTACACTTTCACCAT
CAGCTCTCTTCAACCAGAAGACATTGCAACATATTATTGTCAGCAGTGGA
CTAGTAACCCACCCACGTTCGGTGGAGGGACCAAGCTTGAGATCAAACGG
GAGTTCCCAAAACCCAGCACCCCACCTGGATCAAGCGGAGGAGCACAGGT
CCAGCTCCAGCAGTCCGGTAGCGAACTCAAAAAGCCCGGCGCATCTGTGA
AAGTCAGTTGCAAGGCCTCAGGGTTCACCTTTACAAACTACGGTATGAAT
TGGGTGAAACAGGCTCCCGGGCAGGGTCTGAAGTGGATGGGGTGGATCAA
CACTTACACCAGGGAGCCTACATATGCTGACGATTTCAAAGGTAGATTCG
CATTTTCCCTGGACACAAGCGTGTCCACTGCATACCTGCAGATCAGCTCC
CTCAAGGCCGACGATACTGCTGTGTATTTCTGCGCTAGGGACATTACCGC
AGTGGTCCCAACAGGCTTTGATTATTGGGGCCAGGGATCACTGGTGACTG
TGTCCTCAGGTGAGTCCTTACAACCTCTCTCTTCTATTCAGCTTAAATAG
ATTTTACTGCATTTGTTGGGGGGGAAATGTGTGTATCTGAATTTCAGGTC
ATGAAGGACTAGGGACACCTTGGGAGTCAGAAAGGGTCATTGGGGATCGC
GGCCGCAAGCTT

What is claimed is:

1. A multivalent antibody complex comprising a first and a second polypeptide, each polypeptide comprising V_{H} and V_{L} domains in series, wherein the first and second polypeptides bind to each other to form the antibody complex, wherein a V_{H} domain on one polypeptide binds to a complementary V_{L} domain on the other polypeptide to form an antigen binding site, wherein V_{H} and V_{L} domains on the same polypeptide do not bind to each other and wherein one polypeptide is attached to the amino terminal end of a C_{H}1 domain and the other polypeptide is attached to the amino terminal end of a C_{L} domain.

2. The antibody complex of claim 1, wherein the carboxyl terminal end of the C_{H}1 domain is attached to a C_{H}2-C_{H}3 domain.

3. The antibody complex of claim 1, wherein the first and second polypeptides are fusion proteins.

4. The antibody complex of claim 1, wherein the V_{H} and V_{L} domains on the same polypeptide are joined by linker sequences.

5. The antibody complex of claim 1, wherein the linker sequences are selected from the group consisting of a short flexible linker (SH) and a rigid hinge linker (HL).

6. The antibody complex of claim 1, wherein the antibody complex is a bivalent antibody construct and wherein the first and second polypeptides are selected from the group consisting of:
(a) V_{Ha}-V_{Lb}-C_{H}1 and V_{La}-V_{Hb}-C_{L};
(b) V_{Ha}-V_{Lb}-C_{L} and V_{La}-V_{Hb}-C_{H}1;
(c) ©V_{Ha}-V_{Hb}-C_{H}1 and V_{La}-V_{Lb}-C_{L}; and
(d) V_{Ha}-V_{Hb}-C_{L} and V_{La}-V_{Lb}-C_{H}1.

7. The antibody complex of claim 1, wherein the antibody complex is a trivalent antibody construct and wherein the first and second polypeptides are selected from the group consisting of:
a) V_{Ha}-V_{Lb}-V_{Hc}-C_{H}1 and V_{La}-V_{Hb}-V_{Lc}-C_{L};
b) V_{Ha}-V_{Lb}-V_{Hc}-C_{L} and V_{La}-V_{Hb}-V_{Lc}-C_{H}1;
c) V_{Ha}-V_{Hb}-V_{Hc}-C_{H}1 and V_{La}-V_{Lb}-V_{Lc}-C_{L};
d) V_{Ha}-V_{Hb}-V_{Hc}-C_{L} and V_{La}-V_{Lb}-V_{Lc}-C_{H}1;
e) V_{Ha}-V_{Lb}-V_{Lc}-C_{H}1 and V_{La}-V_{Hb}-V_{Hc}-C_{L};
f) V_{Ha}-V_{Lb}-V_{Lc}-C_{L} and V_{La}-V_{Hb}-V_{Hc}-C_{H}1;
g) V_{Ha}-V_{Hb}-V_{Lc}-C_{H}1 and V_{La}-V_{Lb}-V_{Hc}-C_{L}; and
h) V_{Ha}-V_{Hb}-V_{Lc}-C_{L} and V_{La}-V_{Lb}-V_{Hc}-C_{H}1.

8. The antibody complex of claim 1, wherein the antibody complex is a tetravalent bispecific IgG and wherein the first and second polypeptides are VL2-linker-VH1-CH1-Hinge-CH2-CH3 and VH2-linker-VL1-CL.

9. The antibody complex of claim 1, wherein the antibody complex is a hexavalent monospecific IgG and wherein the first and second polypeptides are VL1-VH1-X-VH1-CH1-Hinge-CH2-CH3 and VH1-VL1-X-VL1-CL.

10. The antibody complex of claim 1, wherein the antibody complex is a hexavalent bispecific IgG and wherein the first and second polypeptides are VL2-VH1-X-VH1-CH1-Hinge-CH2-CH3 and VH2-VL1-X-VL1-CL.

11. The antibody complex of claim 1, wherein the antibody complex is a hexavalent trispecific IgG and wherein the first and second polypeptides are VL3-VH2-X-VH1-CH1-Hinge-CH2-CH3 and VH3-VL2-X-VL1-CL.

12. The antibody complex of claim 1, wherein the antibody complex is a trivalent bispecific IgG and wherein the first and second polypeptides are VL2-VH2-X-VH1-CH1 and VH2-VL2-X-VL1-CL.

13. The antibody complex of claim 1, wherein the antibody complex comprises chimeric, humanized or human antibodies or fragments thereof.

14. The antibody complex of claim 1, wherein the antibody complex comprises human IgG1, IgG2a, IgG3, or IgG4 constant regions.

15. The antibody complex of claim 1, wherein each complementary V_{H} and V_{L} domain bind to an antigen selected from the group consisting of carbonic anhydrase IX, alpha-fetoprotein, α-actinin-4, A3, antigen specific for A33 antibody, ART-4, B7, Ba 733, BAGE, BrE3-antigen, CA125, CAMEL, CAP-1, CASP-8/m, CCCL19, CCCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD45, CD46, CD52, CD54, CD55, CD59, CD64, CD66a-e, CD67, CD70, CD74, CD79a, CD80, CD83, CD95, CD126, CD133, CD138, CD147, CD154, CDC27, CDK-4/m, CDKN2A, CXCR4, colon-specific antigen-p (CSAp), CEA (CEACAM5), CEACAM1, CEACAM6, c-met, DAM, EGFR, EGFRvIII, EGP-1, EGP-2, ELF2-M, Ep-CAM, Flt-1, Flt-3, folate receptor, G250 antigen, GAGE, gp100, GROB, HLA-DR, HM1.24, human chorionic gonadotropin (HCG) and its subunits, HER2/neu, HMGB-1, hypoxia inducible factor (HIF-1), HSP70-2M, HST-2, Ia, IGF-1R, IFN-γ, IFN-α, IFN-β, IL-2, IL-4R, IL-6R, IL-13R, IL-15R, IL-17R, IL-18R, IL-6, IL-8, IL-12, IL-15, IL-17, IL-18, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5, MUM-1/2, MUM-3, NCA66, NCA95, NCA90, antigen specific for PAM-4 antibody, placental growth factor, p53, PLAGL2, prostatic acid phosphatase, PSA, PRAME, PSMA, P1GF, IGF, IGF-1R, IL-6, IL-25, RS5, RANTES, T101, SAGE, 5100, survivin, survivin-2B, TAC, TAG-72, tenascin, TRAIL receptors, TNF-α, Tn antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, Kras, cMET and an oncogene product.

16. The antibody complex of claim 1, wherein each complementary V_{H} and V_{L} domain bind to a lymphocyte antigen selected from the group consisting of CD4, CD5, CD8, CD14, CD15, CD19, CD20, CD21, CD22, CD23, CD25, CD33, CD37, CD38, CD40, CD40L, CD46, CD52, CD54, CD74, CD80, CD126, CD138, CD154, B7, MUC1, Ia, 11, HM1.24, HLA-DR, tenascin, VEGF, P1GF, ED-B fibronectin, an oncogene, an oncogene product, NCA 66a-d, necrosis antigens, IL-2, T101, TAG, IL-6, MW, TRAIL-R1 (DR4) and TRAIL-R2 (DR5).

17. The antibody complex of claim 1, wherein the complementary V_{H} and V_{L} domains are from an antibody selected from the group consisting of J591 (anti-PSMA), hPAM4 (anti-mucin), hA20 (anti-CD20), hA19 (anti-CD19), hIMMU31 (anti-AFP), hLL1 (anti-CD74), hLL2 (anti-CD22), hMu-9 (anti-CSAp), hL243 (anti-HLA-DR), hMN-14 (anti-CEACAM5), hMN-15 (anti-CEACAM6), hR1 (anti-IGF-1R), hRS7 (anti-EGP-1), hMN-3 (anti-CEACAM6), AB-PG1-XG1-026 (anti-PSMA), 29H2 (anti-PSMA), D2/B (anti-PSMA), h679 (anti-HSG) and 734 (anti-DTPA).

18. The antibody complex of claim 1, wherein the first and/or second polypeptide further comprises an AD moiety or a DDD moiety.

19. The antibody complex of claim 18, wherein the first and second polypeptides are selected from the group consisting of:
a) VH₁-VL₂-AD2 and VH₂-VL₁;
b) VH₁-VL₂-AD2 and VH₂-VL₁-AD2;
c) VH₁-VL₂-AD2 and VH₂-VL₁-AD3;
d) VH₁-VH₂-AD2 and VL₁-VL₂;
e) VH₁-VH₂-AD2 and VL₂-VL₁-AD2;
f) VH₁-VH₂-AD2 and VL₂-VL₁-AD3;
g) VH₁-VL₁-VH₂-AD2 and VL₂-VH₁-VL₁;
h) VH₁-VL₁-VH₂-AD2 and VL₂-VH₁-VL₁-AD2;
i) VH₁-VL₁-VH₂-AD2 and VL₂-VH₁-VL₁-AD3;
j) VH₁-CH₁-VH₂-AD2 and VL₁-CL-VL₂;
k) VH₁-CH₁-VH₂-AD2 and VL₁-CL-VL₂-AD2;
l) VH₁-CH₁-VH₂-AD2 and VL₁-CL-VL₂-AD3;
m) VH₁-VH₂-VH₃-AD2 and VL₃-VL₂-VL₁;
n) VH₁-VH₂-VH₃-AD2 and VL₃-VL₂-VL₁-AD2; and
o) VH₁-VH₂-VH₃-AD2 and VL₃-VL₂-VL₁-AD3.

20. The antibody complex of claim 19, further comprising an effector moiety attached to a DDD2 moiety or a DDD3 moiety.

21. The antibody complex of claim 19, further comprising a first effector moiety attached to a DDD2 moiety and a second effector moiety attached to a DDD3 moiety.

22. The antibody complex of claim 18, further comprising a DDD2 or DDD3 moiety attached to the C_{H}1 domain.

23. The antibody complex of claim 22, further comprising an effector moiety attached to an AD2 or AD3 moiety.

24. The antibody complex of claim 18, wherein the amino acid sequence of the AD moiety is selected from the group consisting of SEQ ID NO:3, SEQ ID NO:4, SEQ ID NO:7, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83 and SEQ ID NO:84.

25. The antibody complex of claim 18, wherein the amino acid sequence of the DDD moiety is selected from the group consisting of SEQ ID NO:1, SEQ ID NO:2, SEQ ID NO:5, SEQ ID NO:6, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30 and SEQ ID NO:31.

26. The antibody complex of claim 18, further comprising two or more AD moieties in a single polypeptide.

27. The antibody complex of claim 26, further comprising two or more AD moieties in each polypeptide.

28. The antibody complex of claim 1, wherein the first and second polypeptides are not conjugated to therapeutic or diagnostic agents.

29. The antibody complex of claim 1, wherein the antibody complex is conjugated to at least one therapeutic and/or diagnostic agent.

30. The antibody complex of claim 29, wherein the diagnostic agent is selected from the group consisting of a radioisotope, a dye, a contrast agent, a fluorescent agent, a chemiluminescent agent, a bioluminescent agent, an enhancing agent, a liposome and a paramagnetic ion.

31. The antibody complex of claim 29, wherein the therapeutic agent is selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide, an antisense molecule, a siRNA, a second antibody and a second antibody fragment.

32. The antibody complex of claim 29, wherein the therapeutic agent is selected from the group consisting of aplidin, azaribine, anastrozole, azacytidine, bleomycin, bortezomib, bryostatin-1, busulfan, calicheamycin, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, PSI-341, semustine streptozocin, tamoxifen, taxanes, taxol, testosterone propionate, thalidomide, thioguanine, thiotepa, teniposide, topotecan, uracil mustard, velcade, vinblastine, vinorelbine, vincristine, ricin, abrin, ribonuclease, onconase, rapLRI, DNase I, Staphylococcal enterotoxin-A, pokeweed antiviral protein, gelonin, diphtheria toxin, Pseudomonas exotoxin, and Pseudomonas endotoxin.

33. The antibody complex of claim 29, wherein the therapeutic agent is a radionuclide selected from the group consisting of ^{103m}Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In, ¹¹⁹Sb, ¹¹C, ^{121m}Te, ^{122m}Te, ¹²⁵I, ^{125m}Te, ¹²⁶I, ¹³¹I, ¹³³I, ¹³N, ¹⁴²Pr, ¹⁴³Pr, ¹⁴⁹Pm, ¹⁵²Dy, ¹⁵³Sm, ¹⁵O, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Po, ²¹⁷At, ²¹⁹Rn, ²²¹Fr, ²²³Ra, ²²⁴Ac, ²²⁵Fm, ³²P, ³³P, ⁴⁷Sc, ⁵¹Cr, ⁵⁷Co, ⁵⁸Co, ⁵⁹Fe, ⁶²Cu, ⁶⁷Cu, ⁶⁷Ga, ⁷⁵Br, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo and ^{99m}Tc.

34. The antibody complex of claim 29, wherein the therapeutic agent is an enzyme selected from the group consisting of malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

35. The antibody complex of claim 29, wherein the therapeutic agent is an immunomodulator selected from the group consisting of MIF (macrophage migration inhibitory factor), HMGB-1 (high mobility group box protein 1), erythropoietin, thrombopoietin tumor necrosis factor-α (TNF), TNF-β, granulocyte-colony stimulating factor (G-CSF), granulocyte macrophage-colony stimulating factor (GM-CSF), interferon-α, interferon-β, interferon-γ, interferon-λ, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-β, platelet-growth factor, TGF-α, TGF-β, insulin-like growth factor-I, insulin-like growth factor-II, macrophage-CSF (M-CSF), CCL19, CCL21, IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, MCP-1, RANTES, MIP-1A, MIP-1B, ENA-78, MCP-1, IP-10, Gro-β, Eotaxin, SCF, PDGF, MSF, CNTF, leptin, oncostatin M, EGF, FGF, P1GF, calcitonin, Factor VIII, somatostatin, tissue plasminogen activator, LIF and LT.

36. A method of treating a disease or condition comprising administering to a subject an antibody complex according to claim 1.

37. The method of claim 36, wherein the disease or condition is selected from the group consisting of cancer, autoimmune disease, immune dysregulation disease, organ-graft rejection, graft-versus-host disease, a neurodegenerative disease, a metabolic disease and a cardiovascular disease.

38. The method of claim 37, wherein the cancer is selected from the group consisting of hematopoietic cancer, B-cell leukemia, B-cell lymphoma, non-Hodgkin's lymphoma (NHL), multiple myeloma, chronic lymphocytic leukemia, acute lymphocytic leukemia, acute myelogenous leukemia, glioblastoma, follicular lymphoma. diffuse large B cell lymphoma, colon cancer, pancreatic cancer, renal cancer, lung cancer, stomach cancer, breast cancer, prostate cancer, ovarian cancer and melanoma.

39. The method of claim 37, wherein the autoimmune disease is selected from the group consisting of acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjogren's syndrome, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis and fibrosing alveolitis.

40. The method of claim 36, wherein the first and second polypeptides are not conjugated to any therapeutic agent.

41. The method of claim 40, further comprising administering at least one therapeutic agent to the subject.

42. The method of claim 36, wherein the antibody complex is conjugated to at least one therapeutic agent.

43. The method of claim 42, wherein the therapeutic agent is selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide, an antisense molecule, a siRNA, a second antibody and a second antibody fragment.

44. The method of claim 42, wherein the therapeutic agent is selected from the group consisting of aplidin, azaribine, anastrozole, azacytidine, bleomycin, bortezomib, bryostatin-1, busulfan, calicheamycin, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, PSI-341, semustine streptozocin, tamoxifen, taxanes, taxol, testosterone propionate, thalidomide, thioguanine, thiotepa, teniposide, topotecan, uracil mustard, velcade, vinblastine, vinorelbine, vincristine, ricin, abrin, ribonuclease, onconase, rapLRI, DNase I, Staphylococcal enterotoxin-A, pokeweed antiviral protein, gelonin, diphtheria toxin, Pseudomonas exotoxin, and Pseudomonas endotoxin.

45. The method of claim 42, wherein the therapeutic agent is a radionuclide selected from the group consisting of ^{103m}Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In, ¹¹⁹Sb, ¹¹C, ^{121m}Te, ^{122m}Te, ¹²⁵I, ^{125m}Te, ¹²⁶I, ¹³¹I, ¹³³I, ¹³N, ¹⁴²Pr, ¹⁴³Pr, ¹⁴⁹Pm, ¹⁵²Dy, ¹⁵³Sm, ¹⁵O, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Po, ²¹⁷At, ²¹⁹Rn, ²²¹Fr, ²²³Ra, ²²⁴Ac, ²²⁵Fm, ³²P, ³³P, ⁴⁷Sc, ⁵¹Cr, ⁵⁷Co, ⁵⁸Co, ⁵⁹Fe, ⁶²Cu, ⁶⁷Cu, ⁶⁷Ga, ⁷⁵Br, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo and ^{99m}Tc.

46. The method of claim 42, wherein the therapeutic agent is an enzyme selected from the group consisting of malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

47. The method of claim 42, wherein the therapeutic agent is an immunomodulator selected from the group consisting of MIF (macrophage migration inhibitory factor), HMGB-1 (high mobility group box protein 1), erythropoietin, thrombopoietin tumor necrosis factor-α (TNF), TNF-β, granulocyte-colony stimulating factor (G-CSF), granulocyte macrophage-colony stimulating factor (GM-CSF), interferon-α, interferon-β, interferon-γ, interferon-λ, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-β, platelet-growth factor, TGF-α, TGF-β, insulin-like growth factor-I, insulin-like growth factor-II, macrophage-CSF (M-CSF), CCL19, CCL21, IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, MCP-1, RANTES, MIP-1A, MIP-1B, ENA-78, MCP-1, IP-10, Gro-β, Eotaxin, SCF, PDGF, MSF, CNTF, leptin, oncostatin M, EGF, FGF, P1GF, calcitonin, Factor VIII, somatostatin, tissue plasminogen activator, LIF and LT.


SEQUENCE LISTING (162 sequences)

SEQ ID NO: 1 - protein, 44 aa, Artificial Sequence
  Description of Artificial Sequence Synthetic polypeptide
SHIQIPPGLTELLQGYTVEVLRQQPPDLVEFAVEYFTRLREARA

SEQ ID NO: 2 - protein, 45 aa, Artificial Sequence
  Description of Artificial Sequence Synthetic polypeptide
CGHIQIPPGLTELLQGYTVEVLRQQPPDLVEFAVEYFTRLREARA

SEQ ID NO: 3 - protein, 17 aa, Artificial Sequence
  Description of Artificial Sequence Synthetic peptide
QIEYLAKQIVDNAIQQA

SEQ ID NO: 4 - protein, 21 aa, Artificial Sequence
  Description of Artificial Sequence Synthetic peptide
CGQIEYLAKQIVDNAIQQAGC

SEQ ID NO: 5 - protein, 50 aa, Artificial Sequence
  Description of Artificial Sequence Synthetic polypeptide
SLRECELYVQKHNIQALLKDSIVQLCTARPERPMAFLREYFERLEKEEAK

SEQ ID NO: 6 - protein, 55 aa, Artificial Sequence
  Description of Artificial Sequence Synthetic polypeptide
MSCGGSLRECELYVQKHNIQALLKDSIVQLCTARPERPMAFLREYFERLEKEEAK

SEQ ID NO: 7 - protein, 23 aa, Artificial Sequence
  Description of Artificial Sequence Synthetic peptide
CGFEELAWKIAKMIWSDVFQQGC

SEQ ID NO: 8 - protein, 51 aa, Homo sapiens
SLRECELYVQKHNIQALLKDVSIVQLCTARPERPMAFLREYFEKLEKEEAK

SEQ ID NO: 9 - protein, 54 aa, Homo sapiens
SLKGCELYVQLHGIQQVLKDCIVHLCISKPERPMKFLREHFEKLEKEENRQILA

SEQ ID NO: 10 - protein, 44 aa, Homo sapiens
SHIQIPPGLTELLQGYTVEVGQQPPDLVDFAVEYFTRLREARRQ

SEQ ID NO: 11 - protein, 44 aa, Homo sapiens
SIEIPAGLTELLQGFTVEVLRHQPADLLEFALQHFTRLQQENER

SEQ ID NO: 12 - protein, 44 aa, Artificial Sequence
  Description of Artificial Sequence Synthetic polypeptide
THIQIPPGLTELLQGYTVEVLRQQPPDLVEFAVEYFTRLREARA

SEQ ID NO: 13 - protein, 44 aa, Artificial Sequence
  Description of Artificial Sequence Synthetic polypeptide
SKIQIPPGLTELLQGYTVEVLRQQPPDLVEFAVEYFTRLREARA

SEQ ID NO: 14 - protein, 44 aa, Artificial Sequence
  Description of Artificial Sequence Synthetic polypeptide
SRIQIPPGLTELLQGYTVEVLRQQPPDLVEFAVEYFTRLREARA

SEQ ID NO: 15 - protein, 44 aa, Artificial Sequence
  Description of Artificial Sequence Synthetic polypeptide
SHINIPPGLTELLQGYTVEVLRQQPPDLVEFAVEYFTRLREARA

SEQ ID NO: 16 - protein, 44 aa, Artificial Sequence
  Description of Artificial Sequence Synthetic polypeptide
SHIQIPPALTELLQGYTVEVLRQQPPDLVEFAVEYFTRLREARA

SEQ ID NO: 17 - protein, 44 aa, Artificial Sequence
  Description of Artificial Sequence Synthetic polypeptide
SHIQIPPGLSELLQGYTVEVLRQQPPDLVEFAVEYFTRLREARA

SEQ ID NO: 18 - protein, 44 aa, Artificial Sequence
  Description of Artificial Sequence Synthetic polypeptide
SHIQIPPGLTDLLQGYTVEVLRQQPPDLVEFAVEYFTRLREARA

SEQ ID NO: 19 - protein, 44 aa, Artificial Sequence
  Description of Artificial Sequence Synthetic polypeptide
SHIQIPPGLTELLNGYTVEVLRQQPPDLVEFAVEYFTRLREARA

SEQ ID NO: 20 - protein, 44 aa, Artificial Sequence
  Description of Artificial Sequence Synthetic polypeptide
SHIQIPPGLTELLQAYTVEVLRQQPPDLVEFAVEYFTRLREARA

SEQ ID NO: 21 - protein, 44 aa, Artificial Sequence
  Description of Artificial Sequence Synthetic polypeptide
SHIQIPPGLTELLQGYSVEVLRQQPPDLVEFAVEYFTRLREARA

SEQ ID NO: 22 - protein, 44 aa, Artificial Sequence
  Description of Artificial Sequence Synthetic polypeptide
SHIQIPPGLTELLQGYTVDVLRQQPPDLVEFAVEYFTRLREARA

SEQ ID NO: 23 - protein, 44 aa, Artificial Sequence
  Description of Artificial Sequence Synthetic polypeptide
SHIQIPPGLTELLQGYTVEVLKQQPPDLVEFAVEYFTRLREARA

SEQ ID NO: 24 - protein, 44 aa, Artificial Sequence
  Description of Artificial Sequence Synthetic polypeptide
SHIQIPPGLTELLQGYTVEVLRNQPPDLVEFAVEYFTRLREARA

SEQ ID NO: 25 - protein, 44 aa, Artificial Sequence
  Description of Artificial Sequence Synthetic polypeptide
SHIQIPPGLTELLQGYTVEVLRQNPPDLVEFAVEYFTRLREARA

SEQ ID NO: 26 - protein, 44 aa, Artificial Sequence
  Description of Artificial Sequence Synthetic polypeptide
SHIQIPPGLTELLQGYTVEVLRQQPPELVEFAVEYFTRLREARA

SEQ ID NO: 27 - protein, 44 aa, Artificial Sequence
  Description of Artificial Sequence Synthetic polypeptide
SHIQIPPGLTELLQGYTVEVLRQQPPDLVDFAVEYFTRLREARA

SEQ ID NO: 28 - protein, 44 aa, Artificial Sequence
  Description of Artificial Sequence Synthetic polypeptide
SHIQIPPGLTELLQGYTVEVLRQQPPDLVEFLVEYFTRLREARA

SEQ ID NO: 29 - protein, 44 aa, Artificial Sequence
  Description of Artificial Sequence Synthetic polypeptide
SHIQIPPGLTELLQGYTVEVLRQQPPDLVEFIVEYFTRLREARA

SEQ ID NO: 30 - protein, 44 aa, Artificial Sequence
  Description of Artificial Sequence Synthetic polypeptide
SHIQIPPGLTELLQGYTVEVLRQQPPDLVEFVVEYFTRLREARA

SEQ ID NO: 31 - protein, 44 aa, Artificial Sequence
  Description of Artificial Sequence Synthetic polypeptide
SHIQIPPGLTELLQGYTVEVLRQQPPDLVEFAVDYFTRLREARA

SEQ ID NO: 32 - protein, 17 aa, Artificial Sequence
  Description of Artificial Sequence Synthetic peptide
NIEYLAKQIVDNAIQQA

SEQ ID NO: 33 - protein, 17 aa, Artificial Sequence
  Description of Artificial Sequence Synthetic peptide
QLEYLAKQIVDNAIQQA

SEQ ID NO: 34 - protein, 17 aa, Artificial Sequence
  Description of Artificial Sequence Synthetic peptide
QVEYLAKQIVDNAIQQA

SEQ ID NO: 35 - protein, 17 aa, Artificial Sequence
  Description of Artificial Sequence Synthetic peptide
QIDYLAKQIVDNAIQQA

SEQ ID NO: 36 - protein, 17 aa, Artificial Sequence
  Description of Artificial Sequence Synthetic peptide
QIEFLAKQIVDNAIQQA

SEQ ID NO: 37 - protein, 17 aa, Artificial Sequence
  Description of Artificial Sequence Synthetic peptide
QIETLAKQIVDNAIQQA

SEQ ID NO: 38 - protein, 17 aa, Artificial Sequence
  Description of Artificial Sequence Synthetic peptide
QIESLAKQIVDNAIQQA

SEQ ID NO: 39 - protein, 17 aa, Artificial Sequence
  Description of Artificial Sequence Synthetic peptide
QIEYIAKQIVDNAIQQA

SEQ ID NO: 40 - protein, 17 aa, Artificial Sequence
  Description of Artificial Sequence Synthetic peptide
QIEYVAKQIVDNAIQQA

SEQ ID NO: 41 - protein, 17 aa, Artificial Sequence
  Description of Artificial Sequence Synthetic peptide
QIEYLARQIVDNAIQQA

SEQ ID NO: 42 - protein, 17 aa, Artificial Sequence
  Description of Artificial Sequence Synthetic peptide
QIEYLAKNIVDNAIQQA

SEQ ID NO: 43 - protein, 17 aa, Artificial Sequence
  Description of Artificial Sequence Synthetic peptide
QIEYLAKQIVENAIQQA

SEQ ID NO: 44 - protein, 17 aa, Artificial Sequence
  Description of Artificial Sequence Synthetic peptide
QIEYLAKQIVDQAIQQA

SEQ ID NO: 45 - protein, 17 aa, Artificial Sequence
  Description of Artificial Sequence Synthetic peptide
QIEYLAKQIVDNAINQA

SEQ ID NO: 46 - protein, 17 aa, Artificial Sequence
  Description of Artificial Sequence Synthetic peptide
QIEYLAKQIVDNAIQNA

SEQ ID NO: 47 - protein, 17 aa, Artificial Sequence
  Description of Artificial Sequence Synthetic peptide
QIEYLAKQIVDNAIQQL

SEQ ID NO: 48 - protein, 17 aa, Artificial Sequence
  Description of Artificial Sequence Synthetic peptide
QIEYLAKQIVDNAIQQI

SEQ ID NO: 49 - protein, 17 aa, Artificial Sequence
  Description of Artificial Sequence Synthetic peptide
QIEYLAKQIVDNAIQQV

SEQ ID NO: 50 - protein, 17 aa, Artificial Sequence
  Description of Artificial Sequence Synthetic peptide
QIEYVAKQIVDYAIHQA

SEQ ID NO: 51 - protein, 17 aa, Artificial Sequence
  Description of Artificial Sequence Synthetic peptide
QIEYKAKQIVDHAIHQA

SEQ ID NO: 52 - protein, 17 aa, Artificial Sequence
  Description of Artificial Sequence Synthetic peptide
QIEYHAKQIVDHAIHQA

SEQ ID NO: 53 - protein, 17 aa, Artificial Sequence
  Description of Artificial Sequence Synthetic peptide
QIEYVAKQIVDHAIHQA

SEQ ID NO: 54 - protein, 18 aa, Artificial Sequence
  Description of Artificial Sequence Synthetic peptide
PLEYQAGLLVQNAIQQAI

SEQ ID NO: 55 - protein, 18 aa, Artificial Sequence
  Description of Artificial Sequence Synthetic peptide
LLIETASSLVKNAIQLSI

SEQ ID NO: 56 - protein, 18 aa, Artificial Sequence
  Description of Artificial Sequence Synthetic peptide
LIEEAASRIVDAVIEQVK

SEQ ID NO: 57 - protein, 18 aa, Artificial Sequence
  Description of Artificial Sequence Synthetic peptide
ALYQFADRFSELVISEAL

SEQ ID NO: 58 - protein, 17 aa, Artificial Sequence
  Description of Artificial Sequence Synthetic peptide
LEQVANQLADQIIKEAT

SEQ ID NO: 59 - protein, 17 aa, Artificial Sequence
  Description of Artificial Sequence Synthetic peptide
FEELAWKIAKMIWSDVF

SEQ ID NO: 60 - protein, 18 aa, Artificial Sequence
  Description of Artificial Sequence Synthetic peptide
ELVRLSKRLVENAVLKAV

SEQ ID NO: 61 - protein, 18 aa, Artificial Sequence
  Description of Artificial Sequence Synthetic peptide
TAEEVSARIVQVVTAEAV

SEQ ID NO: 62 - protein, 18 aa, Artificial Sequence
  Description of Artificial Sequence Synthetic peptide
QIKQAAFQLISQVILEAT

SEQ ID NO: 63 - protein, 16 aa, Artificial Sequence
  Description of Artificial Sequence Synthetic peptide
LAWKIAKMIVSDVMQQ

SEQ ID NO: 64 - protein, 24 aa, Artificial Sequence
  Description of Artificial Sequence Synthetic peptide
DLIEEAASRIVDAVIEQVKAAGAY

SEQ ID NO: 65 - protein, 18 aa, Artificial Sequence
  Description of Artificial Sequence Synthetic peptide
LEQYANQLADQIIKEATE

SEQ ID NO: 66 - protein, 20 aa, Artificial Sequence
  Description of Artificial Sequence Synthetic peptide
FEELAWKIAKMIWSDVFQQC

SEQ ID NO: 67 - protein, 17 aa, Artificial Sequence
  Description of Artificial Sequence Synthetic peptide
QIEYLAKQIPDNAIQQA

SEQ ID NO: 68 - protein, 25 aa, Artificial Sequence
  Description of Artificial Sequence Synthetic peptide
KGADLIEEAASRIVDAVIEQVKAAG

SEQ ID NO: 69 - protein, 25 aa, Artificial Sequence
  Description of Artificial Sequence Synthetic peptide
KGADLIEEAASRIPDAPIEQVKAAG

SEQ ID NO: 70 - protein, 25 aa, Artificial Sequence
  Description of Artificial Sequence Synthetic peptide
PEDAELVRLSKRLVENAVLKAVQQY

SEQ ID NO: 71 - protein, 25 aa, Artificial Sequence
  Description of Artificial Sequence Synthetic peptide
PEDAELVRTSKRLVENAVLKAVQQY

SEQ ID NO: 72 - protein, 25 aa, Artificial Sequence
  Description of Artificial Sequence Synthetic peptide
PEDAELVRLSKRDVENAVLKAVQQY

SEQ ID NO: 73 - protein, 25 aa, Artificial Sequence
  Description of Artificial Sequence Synthetic peptide
PEDAELVRLSKRLPENAVLKAVQQY

SEQ ID NO: 74 - protein, 25 aa, Artificial Sequence
  Description of Artificial Sequence Synthetic peptide
PEDAELVRLSKRLPENAPLKAVQQY

SEQ ID NO: 75 - protein, 25 aa, Artificial Sequence
  Description of Artificial Sequence Synthetic peptide
PEDAELVRLSKRLVENAVEKAVQQY

SEQ ID NO: 76 - protein, 25 aa, Artificial Sequence
  Description of Artificial Sequence Synthetic peptide
EEGLDRNEEIKRAAFQIISQVISEA

SEQ ID NO: 77 - protein, 25 aa, Artificial Sequence
  Description of Artificial Sequence Synthetic peptide
LVDDPLEYQAGLLVQNAIQQAIAEQ

SEQ ID NO: 78 - protein, 25 aa, Artificial Sequence
  Description of Artificial Sequence Synthetic peptide
QYETLLIETASSLVKNAIQLSIEQL

SEQ ID NO: 79 - protein, 25 aa, Artificial Sequence
  Description of Artificial Sequence Synthetic peptide
LEKQYQEQLEEEVAKVIVSMSIAFA

SEQ ID NO: 80 - protein, 25 aa, Artificial Sequence
  Description of Artificial Sequence Synthetic peptide
NTDEAQEELAWKIAKMIVSDIMQQA

SEQ ID NO: 81 - protein, 25 aa, Artificial Sequence
  Description of Artificial Sequence Synthetic peptide
VNLDKKAVLAEKIVAEAIEKAEREL

SEQ ID NO: 82 - protein, 25 aa, Artificial Sequence
  Description of Artificial Sequence Synthetic peptide
NGILELETKSSKLVQNIIQTAVDQF

SEQ ID NO: 83 - protein, 25 aa, Artificial Sequence
  Description of Artificial Sequence Synthetic peptide
TQDKNYEDELTQVALALVEDVINYA

SEQ ID NO: 84 - protein, 25 aa, Artificial Sequence
  Description of Artificial Sequence Synthetic peptide
ETSAKDNINIEEAARFLVEKILVNH

SEQ ID NO: 85 - DNA, 21 bp, Artificial Sequence
  Description of Artificial Sequence Synthetic oligonucleotide
aatgcggcggtggtgacagta

SEQ ID NO: 86 - DNA, 21 bp, Artificial Sequence
  Description of Artificial Sequence Synthetic oligonucleotide
aagctcagcacacagaaagac

SEQ ID NO: 87 - DNA, 21 bp, Artificial Sequence
  Description of Artificial Sequence Synthetic oligonucleotide
uaaaaucuuccugcccacctt

SEQ ID NO: 88 - DNA, 21 bp, Artificial Sequence
  Description of Artificial Sequence Synthetic oligonucleotide
ggaagcuguuggcugaaaatt

SEQ ID NO: 89 - RNA, 21 nt, Artificial Sequence
  Description of Artificial Sequence Synthetic oligonucleotide
aagaccagccucuuugcccag

SEQ ID NO: 90 - RNA, 19 nt, Artificial Sequence
  Description of Artificial Sequence Synthetic oligonucleotide
ggaccaggcagaaaacgag

SEQ ID NO: 91 - RNA, 17 nt, Artificial Sequence
  Description of Artificial Sequence Synthetic oligonucleotide
cuaucaggaugacgcgg

SEQ ID NO: 92 - RNA, 21 nt, Artificial Sequence
  Description of Artificial Sequence Synthetic oligonucleotide
ugacacaggcaggcuugacuu

SEQ ID NO: 93 - DNA, 19 bp, Artificial Sequence
  Description of Artificial Sequence Synthetic oligonucleotide
ggtgaagaagggcgtccaa

SEQ ID NO: 94 - DNA, 60 bp, Artificial Sequence
  Description of Artificial Sequence Synthetic oligonucleotide
gatccgttggagctgttggcgtagttcaagagactcgccaacagctccaacttttggaaa

SEQ ID NO: 95 - DNA, 20 bp, Artificial Sequence
  Description of Artificial Sequence Synthetic oligonucleotide
aggtggtgttaacagcagag

SEQ ID NO: 96 - DNA, 21 bp, Artificial Sequence
  Description of Artificial Sequence Synthetic oligonucleotide
aaggtggagcaagcggtggag

SEQ ID NO: 97 - DNA, 21 bp, Artificial Sequence
  Description of Artificial Sequence Synthetic oligonucleotide
aaggagttgaaggccgacaaa

SEQ ID NO: 98 - DNA, 21 bp, Artificial Sequence
  Description of Artificial Sequence Synthetic oligonucleotide
uauggagcugcagaggaugtt

SEQ ID NO: 99 - DNA, 49 bp, Artificial Sequence
  Description of Artificial Sequence Synthetic oligonucleotide
tttgaatatctgtgctgagaacacagttctcagcacagatattcttttt

SEQ ID NO: 100 - DNA, 29 bp, Artificial Sequence
  Description of Artificial Sequence Synthetic oligonucleotide
aatgagaaaagcaaaaggtgccctgtctc

SEQ ID NO: 101 - RNA, 21 nt, Artificial Sequence
  Description of Artificial Sequence Synthetic oligonucleotide
aaucaucaucaagaaagggca

SEQ ID NO: 102 - DNA, 21 bp, Artificial Sequence
  Description of Artificial Sequence Synthetic oligonucleotide
augacugucaggauguugctt

SEQ ID NO: 103 - RNA, 21 nt, Artificial Sequence
  Description of Artificial Sequence Synthetic oligonucleotide
gaacgaauccugaagacaucu

SEQ ID NO: 104 - DNA, 29 bp, Artificial Sequence
  Description of Artificial Sequence Synthetic oligonucleotide
aagcctggctacagcaatatgcctgtctc

SEQ ID NO: 105 - DNA, 21 bp, Artificial Sequence
  Description of Artificial Sequence Synthetic oligonucleotide
ugaccaucaccgaguuuautt

SEQ ID NO: 106 - DNA, 21 bp, Artificial Sequence
  Description of Artificial Sequence Synthetic oligonucleotide
aagtcggacgcaacagagaaa

SEQ ID NO: 107 - DNA, 21 bp, Artificial Sequence
  Description of Artificial Sequence Synthetic oligonucleotide
cuaccuuucuacggacgugtt

SEQ ID NO: 108 - DNA, 21 bp, Artificial Sequence
  Description of Artificial Sequence Synthetic oligonucleotide
ctgcctaaggcggatttgaat

SEQ ID NO: 109 - DNA, 21 bp, Artificial Sequence
  Description of Artificial Sequence Synthetic oligonucleotide
ttauuccuucuucgggaaguc

SEQ ID NO: 110 - DNA, 21 bp, Artificial Sequence
  Description of Artificial Sequence Synthetic oligonucleotide
aaccttctggaacccgcccac

SEQ ID NO: 111 - DNA, 19 bp, Artificial Sequence
  Description of Artificial Sequence Synthetic oligonucleotide
gagcatcttcgagcaagaa

SEQ ID NO: 112 - DNA, 19 bp, Artificial Sequence
  Description of Artificial Sequence Synthetic oligonucleotide
catgtggcaccgtttgcct

SEQ ID NO: 113 - DNA, 21 bp, Artificial Sequence
  Description of Artificial Sequence Synthetic oligonucleotide
aactaccagaaaggtatacct

SEQ ID NO: 114 - DNA, 21 bp, Artificial Sequence
  Description of Artificial Sequence Synthetic oligonucleotide
ucacaguguccuuuauguatt

SEQ ID NO: 115 - DNA, 21 bp, Artificial Sequence
  Description of Artificial Sequence Synthetic oligonucleotide
gcaugaaccggaggcccautt

SEQ ID NO: 116 - DNA, 19 bp, Artificial Sequence
  Description of Artificial Sequence Synthetic oligonucleotide
ccggacagttccatgtata

SEQ ID NO: 117 - protein, 44 aa, Artificial Sequence
  Description of Artificial Sequence Synthetic polypeptide
XXIXIPPXLXXLLXXYXVXVLXXXPPXLVXFXVXYFXXLXXXXX

SEQ ID NO: 118 - protein, 17 aa, Artificial Sequence
  Description of Artificial Sequence Synthetic peptide
XXXXXAXXIVXXAIXXX

SEQ ID NO: 119 - protein, 44 aa, Artificial Sequence
  Description of Artificial Sequence Synthetic polypeptide
XHIXIPPGLXELLQGYTXEVLRXQPPDLVEFAXXYFXXLXEXRX

SEQ ID NO: 120 - protein, 368 aa, Artificial Sequence
  Description of Artificial Sequence Synthetic polypeptide
MGWSCIILFLVATATGVHSQVQLQQSGSELKKPGASVKVSCKASGFTFTNYGMNWVKQAPGQGLKWMGWINTYTREPTYADDFKGRFAFSLDTSVSTAYLQISSLKADDTAVYFCARDITAVVPTGFDYWGQGSLVTVSSEFPKPSTPPGSSGGADIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 593 aa, Artificial Sequence
  Description of Artificial Sequence Synthetic polypeptide
MGWSCIILFLVATATGVHSDIQLTQSPSSLSASVGDRVTITCRASENIYSNLAWYRQKPGKAPKLLVFAASNLADGVPSRFSGSGSGTDYTFTISSLQPEDIATYYCQHFWTTPWAFGGGTKLQIKREFPKPSTPPGSSGGAQVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - DNA, 965 bp, Artificial Sequence
  Description of Artificial Sequence Synthetic polynucleotide
ctcgagcacacaggacctcaccatgggatggtcatgtattatcctctttctcgtggcaacagcaacaggcgtccatagtgatattcagctcacacagtccccttcttctctcagcgccagcgtgggcgacagggtcactatcacctgcagagcatcagagaacatctacagcaacctggcctggtatcgacagaagcctggcaaagctccaaagctgctcgtgttcgccgcttccaacctcgctgatggagtccccagcaggttcagcggaagcggatccggtactgactacaccttcaccatcagctccctgcagcccgaggatattgctacctactattgccagcacttctggaccacaccttgggcatttggcggagggactaaactgcagatcaagagggagttcccaaaacccagcaccccacctggatcaagcggaggagcacaggtgcagctccagcagagtggcgctgaagtcaagaaacccggatcctctgtgaaagtcagctgtaaggcctccggctacaccttcaccagctataacatgcactgggtgaagcaggcacctgggcagggtctggagtggatcggagccatctacccaggcaacggagacacctcctataatcagaagttcaaagggaaggcaaccctcacagccgatgaatctactaataccgcttacatggagctgagttcactccggtctgaagatacagccttttactattgtgctcgcagtacttactacgggggggattggtacttcgacgtgtggggtcagggaactactgtcactgtgtcctcaggtgagtccttacaacctctctcttctattcagcttaaatagattttactgcatttgttgggggggaaatgtgtgtatctgaatttcaggtcatgaaggactagggacaccttgggagtcagaaagggtcattggggatcgcggccgcaagctt

SEQ ID NO: 123 - DNA, 640 bp, Artificial Sequence
  Description of Artificial Sequence Synthetic polynucleotide
tctagacacaggacctcaccatgggatggagttgtattattctctttctggtcgctaccgctaccggcgtgcattcccaggtccagctccagcagtccggtagcgaactcaaaaagcccggcgcatctgtgaaagtcagttgcaaggcctcagggttcacctttacaaactacggtatgaattgggtgaaacaggctcccgggcagggtctgaagtggatggggtggatcaacacttacaccagggagcctacatatgctgacgatttcaaaggtagattcgcattttccctggacacaagcgtgtccactgcatacctgcagatcagctccctcaaggccgacgatactgctgtgtatttctgcgctagggacattaccgcagtggtcccaacaggctttgattattggggccagggatcactggtgactgtctctagtgaatttccaaaacccagtaccccacctgggtctagtggtggagcagacattcagctgacacagagcccctcaagcctctctgcaagtgtgggcgaccgggtcaccatgacatgtcgcgcctcctctagtgtgtcctacattcactggtttcagcagaagcccggtaaagcccctaagccttggatctacgccacttcgaa

SEQ ID NO: 124 - protein, 480 aa, Artificial Sequence
  Description of Artificial Sequence Synthetic polypeptide
MGWSCIILFLVATATGVHSDIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRGGGGSQVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSEFPKPSTPPGSSGGADIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 718 aa, Artificial Sequence
  Description of Artificial Sequence Synthetic polypeptide
MGWSCIILFLVATATGVHSQVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSGGGGSDIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKREFPKPSTPPGSSGGAQVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - DNA, 976 bp, Artificial Sequence
  Description of Artificial Sequence Synthetic polynucleotide
tctagacacaggacctcaccatgggatggagttgtattattctctttctggtcgctaccgctaccggcgtgcattccgacattcagctgacacagagcccctcaagcctctctgcaagtgtgggcgaccgggtcaccatgacatgtcgcgcctcctctagtgtgtcctacattcactggtttcagcagaagcccggtaaagcccctaagccttggatctacgccacttcaaacctggcttctggtgtccctgtccgattctctggcagcggatctgggacagattacactttcaccatcagctctcttcaaccagaagacattgcaacatattattgtcagcagtggactagtaacccacccacgttcggtggagggaccaagcttcagatcacccggggaggtggaggctcacaggtgcagctccagcagagtggcgctgaagtcaagaaacccggatcctctgtgaaagtcagctgtaaggcctccggctacaccttcaccagctataacatgcactgggtgaagcaggcacctgggcagggtctggagtggatcggagccatctacccaggcaacggagacacctcctataatcagaagttcaaagggaaggcaaccctcacagccgatgaatctactaataccgcttacatggagctgagttcactccggtctgaagatacagccttttactattgtgctcgcagtacttactacgggggggattggtacttcgacgtgtggggtcagggaactactgtcactgtgtcctcagaatttccaaaacccagtaccccacctgggtctagtggtggagcagacattcagctgacacagagcccctcaagcctctctgcaagtgtgggcgaccgggtcaccatgacatgtcgcgcctcctctagtgtgtcctacattcactggtttcagcagaagcccggtaaagcccctaagccttggatctacgccacttcgaa

SEQ ID NO: 127 - DNA, 1340 bp, Artificial Sequence
  Description of Artificial Sequence Synthetic polynucleotide
ctcgagcacacaggacctcaccatgggatggtcatgtattatcctctttctcgtggcaacagcaacaggcgtccatagtcaggtgcagctccagcagagtggcgctgaagtcaagaaacccggatcctctgtgaaagtcagctgtaaggcctccggctacaccttcaccagctataacatgcactgggtgaagcaggcacctgggcagggtctggagtggatcggagccatctacccaggcaacggagacacctcctataatcagaagttcaaagggaaggcaaccctcacagccgatgaatctactaataccgcttacatggagctgagttcactccggtctgaagatacagccttttactattgtgctcgcagtacttactacgggggggattggtacttcgacgtgtggggtcagggaactactgtcactgtgtcctcaggaggtggaggctcagacattcagctgacacagagcccctcaagcctctctgcaagtgtgggcgaccgggtcaccatgacatgtcgcgcctcctctagtgtgtcctacattcactggtttcagcagaagcccggtaaagcccctaagccttggatctacgccacttcaaacctggcttctggtgtccctgtccgattctctggcagcggatctgggacagattacactttcaccatcagctctcttcaaccagaagacattgcaacatattattgtcagcagtggactagtaacccacccacgttcggtggagggaccaagcttcagatcacccgggagttcccaaaacccagcaccccacctggatcaagcggaggagcacaggtgcagctccagcagagtggcgctgaagtcaagaaacccggatcctctgtgaaagtcagctgtaaggcctccggctacaccttcaccagctataacatgcactgggtgaagcaggcacctgggcagggtctggagtggatcggagccatctacccaggcaacggagacacctcctataatcagaagttcaaagggaaggcaaccctcacagccgatgaatctactaataccgcttacatggagctgagttcactccggtctgaagatacagccttttactattgtgctcgcagtacttactacgggggggattggtacttcgacgtgtggggtcagggaactactgtcactgtgtcctcaggtgagtccttacaacctctctcttctattcagcttaaatagattttactgcatttgttgggggggaaatgtgtgtatctgaatttcaggtcatgaaggactagggacaccttgggagtcagaaagggtcattggggatcgcggccgcaagctt

SEQ ID NO: 128 - protein, 480 aa, Artificial Sequence
  Description of Artificial Sequence Synthetic polypeptide
MGWSCIILFLVATATGVHSDIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRGGGGSQVQLQQSGSELKKPGASVKVSCKASGFTFTNYGMNWVKQAPGQGLKWMGWINTYTREPTYADDFKGRFAFSLDTSVSTAYLQISSLKADDTAVYFCARDITAVVPTGFDYWGQGSLVTVSSEFPKPSTPPGSSGGADIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 719 aa, Artificial Sequence
  Description of Artificial Sequence Synthetic polypeptide
MGWSCIILFLVATATGVHSQVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSGGGGSDIQLTQSPSSLSASVGDRVTITCRASENIYSNLAWYRQKPGKAPKLLVFAASNLADGVPSRFSGSGSGTDYTFTISSLQPEDIATYYCQHFWTTPWAFGGGTKLQIKREFPKPSTPPGSSGGAQVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - DNA, 976 bp, Artificial Sequence
  Description of Artificial Sequence Synthetic polynucleotide
tctagacacaggacctcaccatgggatggagttgtattattctctttctggtcgctaccgctaccggcgtgcattccgacattcagctgacacagagcccctcaagcctctctgcaagtgtgggcgaccgggtcaccatgacatgtcgcgcctcctctagtgtgtcctacattcactggtttcagcagaagcccggtaaagcccctaagccttggatctacgccacttcaaacctggcttctggtgtccctgtccgattctctggcagcggatctgggacagattacactttcaccatcagctctcttcaaccagaagacattgcaacatattattgtcagcagtggactagtaacccacccacgttcggtggagggaccaagcttcagatcacccggggaggtggaggctcacaggtccagctccagcagtccggtagcgaactcaaaaagcccggcgcatctgtgaaagtcagttgcaaggcctcagggttcacctttacaaactacggtatgaattgggtgaaacaggctcccgggcagggtctgaagtggatggggtggatcaacacttacaccagggagcctacatatgctgacgatttcaaaggtagattcgcattttccctggacacaagcgtgtccactgcatacctgcagatcagctccctcaaggccgacgatactgctgtgtatttctgcgctagggacattaccgcagtggtcccaacaggctttgattattggggccagggatcactggtgactgtctctagtgaatttccaaaacccagtaccccacctgggtctagtggtggagcagacattcagctgacacagagcccctcaagcctctctgcaagtgtgggcgaccgggtcaccatgacatgtcgcgcctcctctagtgtgtcctacattcactggtttcagcagaagcccggtaaagcccctaagccttggatctacgccacttcgaa

SEQ ID NO: 131 - DNA, 1343 bp, Artificial Sequence
  Description of Artificial Sequence Synthetic polynucleotide
ctcgagcacacaggacctcaccatgggatggtcatgtattatcctctttctcgtggcaacagcaacaggcgtccatagtcaggtgcagctccagcagagtggcgctgaagtcaagaaacccggatcctctgtgaaagtcagctgtaaggcctccggctacaccttcaccagctataacatgcactgggtgaagcaggcacctgggcagggtctggagtggatcggagccatctacccaggcaacggagacacctcctataatcagaagttcaaagggaaggcaaccctcacagccgatgaatctactaataccgcttacatggagctgagttcactccggtctgaagatacagccttttactattgtgctcgcagtacttactacgggggggattggtacttcgacgtgtggggtcagggaactactgtcactgtgtcctcaggaggtggaggctcagatattcagctcacacagtccccttcttctctcagcgccagcgtgggcgacagggtcactatcacctgcagagcatcagagaacatctacagcaacctggcctggtatcgacagaagcctggcaaagctccaaagctgctcgtgttcgccgcttccaacctcgctgatggagtccccagcaggttcagcggaagcggatccggtactgactacaccttcaccatcagctccctgcagcccgaggatattgctacctactattgccagcacttctggaccacaccttgggcatttggcggagggactaaactgcagatcaagagggagttcccaaaacccagcaccccacctggatcaagcggaggagcacaggtgcagctccagcagagtggcgctgaagtcaagaaacccggatcctctgtgaaagtcagctgtaaggcctccggctacaccttcaccagctataacatgcactgggtgaagcaggcacctgggcagggtctggagtggatcggagccatctacccaggcaacggagacacctcctataatcagaagttcaaagggaaggcaaccctcacagccgatgaatctactaataccgcttacatggagctgagttcactccggtctgaagatacagccttttactattgtgctcgcagtacttactacgggggggattggtacttcgacgtgtggggtcagggaactactgtcactgtgtcctcaggtgagtccttacaacctctctcttctattcagcttaaatagattttactgcatttgttgggggggaaatgtgtgtatctgaatttcaggtcatgaaggactagggacaccttgggagtcagaaagggtcattggggatcgcggccgcaagctt

SEQ ID NO: 132 - protein, 480 aa, Artificial Sequence
  Description of Artificial Sequence Synthetic polypeptide
MGWSCIILFLVATATGVHSDIQLTQSPSSLSASVGDRVTMSCRASQSVSYMNWYQQKPGKAPKLWIYDTSKVASGVPSRFSGSGSGTDYTFTISSLQPEDIATYYCQQNSSNPLTFGGGTKVQIKRGGGGSQVQLQQSGSELKKPGASVKVSCKASGFTFTNYGMNWVKQAPGQGLKWMGWINTYTREPTYADDFKGRFAFSLDTSVSTAYLQISSLKADDTAVYFCARDITAVVPTGFDYWGQGSLVTVSSEFPKPSTPPGSSGGADIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 717 aa, Artificial Sequence
  Description of Artificial Sequence Synthetic polypeptide
MGWSCIILFLVATATGVHSQVQLQQSGAEVKKPGSSVKVSCKASGYTFTRYTMHWVRQAPGQGLEWIGYINPSRGYTNYADSVKGKATITADESTNTAYMELSSLRSEDTAFYYCARYYDDHYCLDYWGQGTTVTVSSGGGGSDIQLTQSPSSLSASVGDRVTITCRASENIYSNLAWYRQKPGKAPKLLVFAASNLADGVPSRFSGSGSGTDYTFTISSLQPEDIATYYCQHFWTTPWAFGGGTKLQIKREFPKPSTPPGSSGGAQVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - DNA, 976 bp, Artificial Sequence
  Description of Artificial Sequence Synthetic polynucleotide
tctagacacaggacctcaccatgggatggagttgtattattctctttctggtcgctaccgctaccggcgtgcattccgatattcagctgacacagtctccatcttccctcagtgcctccgtgggcgatagagtcacaatgtcctgccgggcttcacagtccgtgagctacatgaactggtatcagcagaagcccggtaaagcccctaagctctggatctacgacaccagcaaagtggcttccggagtcccatctaggttctctggcagtggatcagggactgactacacctttacaatcagctccctgcagcccgaggatattgccacctactattgccagcagaacagcagcaacccactgacttttggcggaggaactaaggtccagattaagaggggaggtggaggctcacaggtccagctccagcagtccggtagcgaactcaaaaagcccggcgcatctgtgaaagtcagttgcaaggcctcagggttcacctttacaaactacggtatgaattgggtgaaacaggctcccgggcagggtctgaagtggatggggtggatcaacacttacaccagggagcctacatatgctgacgatttcaaaggtagattcgcattttccctggacacaagcgtgtccactgcatacctgcagatcagctccctcaaggccgacgatactgctgtgtatttctgcgctagggacattaccgcagtggtcccaacaggctttgattattggggccagggatcactggtgactgtctctagtgaatttccaaaacccagtaccccacctgggtctagtggtggagcagacattcagctgacacagagcccctcaagcctctctgcaagtgtgggcgaccgggtcaccatgacatgtcgcgcctcctctagtgtgtcctacattcactggtttcagcagaagcccggtaaagcccctaagccttggatctacgccacttcgaa

SEQ ID NO: 135 - DNA, 1337 bp, Artificial Sequence
  Description of Artificial Sequence Synthetic polynucleotide
ctcgagcacacaggacctcaccatgggatggtcatgtattatcctctttctcgtggcaacagcaacaggcgtccatagtcaggtccagctgcagcagtccggggcagaggtcaagaagccaggcagcagcgtcaaggtgtcctgtaaggcaagcggttatacttttacaaggtacactatgcactgggtgagacaggcaccaggacagggactggagtggatcgggtatattaacccttctaggggttacaccaattatgctgacagtgtcaagggaaaagccaccatcacagctgatgaaagcactaacaccgcatacatggagctgagctccctccggtctgaagacacagcattctattactgcgcccgctattacgacgaccattactgtctggactactgggggcaggggactacggtcaccgtctcctcaggaggtggaggctcagatattcagctcacacagtccccttcttctctcagcgccagcgtgggcgacagggtcactatcacctgcagagcatcagagaacatctacagcaacctggcctggtatcgacagaagcctggcaaagctccaaagctgctcgtgttcgccgcttccaacctcgctgatggagtccccagcaggttcagcggaagcggatccggtactgactacaccttcaccatcagctccctgcagcccgaggatattgctacctactattgccagcacttctggaccacaccttgggcatttggcggagggactaaactgcagatcaagagggagttcccaaaacccagcaccccacctggatcaagcggaggagcacaggtgcagctccagcagagtggcgctgaagtcaagaaacccggatcctctgtgaaagtcagctgtaaggcctccggctacaccttcaccagctataacatgcactgggtgaagcaggcacctgggcagggtctggagtggatcggagccatctacccaggcaacggagacacctcctataatcagaagttcaaagggaaggcaaccctcacagccgatgaatctactaataccgcttacatggagctgagttcactccggtctgaagatacagccttttactattgtgctcgcagtacttactacgggggggattggtacttcgacgtgtggggtcagggaactactgtcactgtgtcctcaggtgagtccttacaacctctctcttctattcagcttaaatagattttactgcatttgttgggggggaaatgtgtgtatctgaatttcaggtcatgaaggactagggacaccttgggagtcagaaagggtcattggggatcgcggccgcaagctt

SEQ ID NO: 136 - protein, 467 aa, Artificial Sequence
  Description of Artificial Sequence Synthetic polypeptide
MGWSCIILFLVATATGVHSDIQLTQSPSSLSASVGDRVTMSCRASQSVSYMNWYQQKPGKAPKLWIYDTSKVASGVPSRFSGSGSGTDYTFTISSLQPEDIATYYCQQNSSNPLTFGGGTKVQIKRGGGGSDIQLTQSPSSLSASVGDRVTITCRASENIYSNLAWYRQKPGKAPKLLVFAASNLADGVPSRFSGSGSGTDYTFTISSLQPEDIATYYCQHFWTTPWAFGGGTKLQIKREFPKPSTPPGSSGGADIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 730 aa, Artificial Sequence
  Description of Artificial Sequence Synthetic polypeptide
MGWSCIILFLVATATGVHSQVQLQQSGAEVKKPGSSVKVSCKASGYTFTRYTMHWVRQAPGQGLEWIGYINPSRGYTNYADSVKGKATITADESTNTAYMELSSLRSEDTAFYYCARYYDDHYCLDYWGQGTTVTVSSGGGGSQVQLQQSGSELKKPGASVKVSCKASGFTFTNYGMNWVKQAPGQGLKWMGWINTYTREPTYADDFKGRFAFSLDTSVSTAYLQISSLKADDTAVYFCARDITAVVPTGFDYWGQGSLVTVSSEFPKPSTPPGSSGGAQVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 492 aa, Artificial Sequence
  Description of Artificial Sequence Synthetic polypeptide
MGWSCIILFLVATATGVHSQVQLQQSGAEVKKPGSSVKVSCKASGYTFTRYTMHWVRQAPGQGLEWIGYINPSRGYTNYADSVKGKATITADESTNTAYMELSSLRSEDTAFYYCARYYDDHYCLDYWGQGTTVTVSSGGGGSQVQLQQSGSELKKPGASVKVSCKASGFTFTNYGMNWVKQAPGQGLKWMGWINTYTREPTYADDFKGRFAFSLDTSVSTAYLQISSLKADDTAVYFCARDITAVVPTGFDYWGQGSLVTVSSEFPKPSTPPGSSGGADIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 705 aa, Artificial Sequence
  Description of Artificial Sequence Synthetic polypeptide
MGWSCIILFLVATATGVHSDIQLTQSPSSLSASVGDRVTMSCRASQSVSYMNWYQQKPGKAPKLWIYDTSKVASGVPSRFSGSGSGTDYTFTISSLQPEDIATYYCQQNSSNPLTFGGGTKVQIKRGGGGSDIQLTQSPSSLSASVGDRVTITCRASENIYSNLAWYRQKPGKAPKLLVFAASNLADGVPSRFSGSGSGTDYTFTISSLQPEDIATYYCQHFWTTPWAFGGGTKLQIKREFPKPSTPPGSSGGAQVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 494 aa, Artificial Sequence
  Description of Artificial Sequence Synthetic polypeptide
MGWSCIILFLVATATGVHSDIQLTQSPSSLSASVGDRVTMSCRASQSVSYMNWYQQKPGKAPKLWIYDTSKVASGVPSRFSGSGSGTDYTFTISSLQPEDIATYYCQQNSSNPLTFGGGTKVQIKRGGGGSQVQLQQSGSELKKPGASVKVSCKASGFTFTNYGMNWVKQAPGQGLKWMGWINTYTREPTYADDFKGRFAFSLDTSVSTAYLQISSLKADDTAVYFCARDITAVVPTGFDYWGQGSLVTVSSEFPKPSTPPGSSGGAQVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 703 aa, Artificial Sequence
  Description of Artificial Sequence Synthetic polypeptide
MGWSCIILFLVATATGVHSQVQLQQSGAEVKKPGSSVKVSCKASGYTFTRYTMHWVRQAPGQGLEWIGYINPSRGYTNYADSVKGKATITADESTNTAYMELSSLRSEDTAFYYCARYYDDHYCLDYWGQGTTVTVSSGGGGSDIQLTQSPSSLSASVGDRVTITCRASENIYSNLAWYRQKPGKAPKLLVFAASNLADGVPSRFSGSGSGTDYTFTISSLQPEDIATYYCQHFWTTPWAFGGGTKLQIKREFPKPSTPPGSSGGADIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 480 aa, Artificial Sequence
  Description of Artificial Sequence Synthetic polypeptide
MGWSCIILFLVATATGVHSDIQLTQSPSSLSASVGDRVTMSCRASQSVSYMNWYQQKPGKAPKLWIYDTSKVASGVPSRFSGSGSGTDYTFTISSLQPEDIATYYCQQNSSNPLTFGGGTKVQIKRGGGGSQVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSEFPKPSTPPGSSGGADIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 487 aa, Artificial Sequence
  Description of Artificial Sequence Synthetic polypeptide
MGWSCIILFLVATATGVHSQVQLQQSGAEVKKPGSSVKVSCKASGYTFTRYTMHWVRQAPGQGLEWIGYINPSRGYTNYADSVKGKATITADESTNTAYMELSSLRSEDTAFYYCARYYDDHYCLDYWGQGTTVTVSSGGGGSDIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKREFPKPSTPPGSSGGAQVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKPKSC

SEQ ID NO: 144 - DNA, 957 bp, Artificial Sequence
  Description of Artificial Sequence Synthetic polynucleotide
tgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaggatccggaggtggcgggtctggcggatgtggccagatcgagtacctggccaagcagatcgtggacaacgccatccagcaggccggctgctgaactagtgcggccggcaagcccccgctccccgggctctcgcggtcgcacgaggatgcttggcacgtaccccgtctacatacttcccaggcacccagcatggaaataaagcacccaccactgccctgggcccctgcgagactgtgatggttctttccacgggtcaggccgagtctgaggcctgagtggcatgagggaggcagagcgggtcccactgtccccacactggcccaggctgtgcaggtgtgcctgggccgcctagggtggggctcagccaggggctgccctcggcagggtgggggatttgccagcgtggccctccctccagcagcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatg

SEQ ID NO: 145 - protein, 623 aa, Artificial Sequence
  Description of Artificial Sequence Synthetic polypeptide
MGWSCIILFLVATATGVHSDIQLTQSPSSLSASVGDRVTITCRASENIYSNLAWYRQKPGKAPKLLVFAASNLADGVPSRFSGSGSGTDYTFTISSLQPEDIATYYCQHFWTTPWAFGGGTKLQIKREFPKPSTPPGSSGGAQVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGCGQIEYLAKQIVDNAIQQAGC

SEQ ID NO: 146 - DNA, 382 bp, Artificial Sequence
  Description of Artificial Sequence Synthetic polynucleotide
accggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaacctaagagctgcggcggcggaggatccggaggtggcgggtctggcggaggttgcggccacatccagatcccgccggggctcacggagctgctgcagggctacacggtggaggtgctgcgacagcagccgcctgacctcgtcgaattcgcagtggagtacttcacccgcctgagagaagctcgcgcttgacggccg

SEQ ID NO: 147 - protein, 422 aa, Artificial Sequence
  Description of Artificial Sequence Synthetic polypeptide
MGWSCIILFLVATATGVHSDIQLTQSPSSLSASVGDRVTITCRASENIYSNLAWYRQKPGKAPKLLVFAASNLADGVPSRFSGSGSGTDYTFTISSLQPEDIATYYCQHFWTTPWAFGGGTKLQIKREFPKPSTPPGSSGGAQVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKPKSCGGGGSGGGGSGGGCGHIQIPPGLTELLQGYTVEVLRQQPPDLVEFAVEYFTRLREARA

SEQ ID NO: 148 - protein, 55 aa, Artificial Sequence
  Description of Artificial Sequence Synthetic polypeptide
GSGGGGSGGGGSHIQIPPGLTELLQGYTVEVLRQQPPDLVEFAVEYFTRLREARA

SEQ ID NO: 149 - protein, 29 aa, Artificial Sequence
  Description of Artificial Sequence Synthetic peptide
GSGGGGSGGGGSQIEYLAKQIVDNAIQQA

SEQ ID NO: 150 - protein, 10 aa, Artificial Sequence
  Description of Artificial Sequence Synthetic peptide
GGGGSGGGCG

SEQ ID NO: 151 - DNA, 24 bp, Artificial Sequence
  Description of Artificial Sequence Synthetic primer
agatctggcgcacctgaactcctg

SEQ ID NO: 152 - DNA, 33 bp, Artificial Sequence
  Description of Artificial Sequence Synthetic primer
gaattcggatcctttacccggagacagggagag

SEQ ID NO: 153 - protein, 369 aa, Artificial Sequence
  Description of Artificial Sequence Synthetic polypeptide
MGWSCIILFLVATATGVHSQVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSEFPKPSTPPGSSGGADIQLTQSPSSLSASVGDRVTITCRASENIYSNLAWYRQKPGKAPKLLVFAASNLADGVPSRFSGSGSGTDYTFTISSLQPEDIATYYCQHFWTTPWAFGGGTKLQIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 622 aa, Artificial Sequence
  Description of Artificial Sequence Synthetic polypeptide
MGWSCIILFLVATATGVHSDIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKREFPKPSTPPGSSGGAQVQLQQSGSELKKPGASVKVSCKASGFTFTNYGMNWVKQAPGQGLKWMGWINTYTREPTYADDFKGRFAFSLDTSVSTAYLQISSLKADDTAVYFCARDITAVVPTGFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGCGQIEYLAKQIVDNAIQQAGC

SEQ ID NO: 155 - DNA, 668 bp, Artificial Sequence
  Description of Artificial Sequence Synthetic polynucleotide
tctagacacaggacctcaccatgggatggagttgtattattctctttctggtcgctaccgctaccggcgtgcattcccaggtgcagctccagcagagtggcgctgaagtcaagaaacccggatcctctgtgaaagtcagctgtaaggcctccggctacaccttcaccagctataacatgcactgggtgaagcaggcacctgggcagggtctggagtggatcggagccatctacccaggcaacggagacacctcctataatcagaagttcaaagggaaggcaaccctcacagccgatgaatctactaataccgcttacatggagctgagttcactccggtctgaagatacagccttttactattgtgctcgcagtacttactacgggggggattggtacttcgacgtgtggggtcagggaactactgtcactgtgtcctcagaatttccaaaacccagtaccccacctgggtctagtggtggagcagacatccagctgacccagtctccatcatctctgagcgcatctgttggagatagggtcactatcacttgtcgagcaagtgagaatatttacagtaatttagcatggtatcgtcagaaaccagggaaagcacctaaactgctggtctttgctgcatcaaacttagcagatggtgtgccttcgcga

SEQ ID NO: 156 - DNA, 962 bp, Artificial Sequence
  Description of Artificial Sequence Synthetic polynucleotide
ctcgagcacacaggacctcaccatgggatggtcatgtattatcctctttctcgtggcaacagcaacaggcgtccatagtgacattcagctgacacagagcccctcaagcctctctgcaagtgtgggcgaccgggtcaccatgacatgtcgcgcctcctctagtgtgtcctacattcactggtttcagcagaagcccggtaaagcccctaagccttggatctacgccacttcaaacctggcttctggtgtccctgtccgattctctggcagcggatctgggacagattacactttcaccatcagctctcttcaaccagaagacattgcaacatattattgtcagcagtggactagtaacccacccacgttcggtggagggaccaagcttgagatcaaacgggagttcccaaaacccagcaccccacctggatcaagcggaggagcacaggtccagctccagcagtccggtagcgaactcaaaaagcccggcgcatctgtgaaagtcagttgcaaggcctcagggttcacctttacaaactacggtatgaattgggtgaaacaggctcccgggcagggtctgaagtggatggggtggatcaacacttacaccagggagcctacatatgctgacgatttcaaaggtagattcgcattttccctggacacaagcgtgtccactgcatacctgcagatcagctccctcaaggccgacgatactgctgtgtatttctgcgctagggacattaccgcagtggtcccaacaggctttgattattggggccagggatcactggtgactgtgtcctcaggtgagtccttacaacctctctcttctattcagcttaaatagattttactgcatttgttgggggggaaatgtgtgtatctgaatttcaggtcatgaaggactagggacaccttgggagtcagaaagggtcattggggatcgcggccgcaagctt

SEQ ID NO: 157 - protein, 15 aa, Artificial Sequence
  Description of Artificial Sequence Synthetic peptide
EFPKPSTPPGSSGGA

SEQ ID NO: 158 - protein, 5 aa, Artificial Sequence
  Description of Artificial Sequence Synthetic peptide
GGGGS

SEQ ID NO: 159 - protein, 9 aa, Artificial Sequence
  Description of Artificial Sequence Synthetic peptide
GSGGGGSGG

SEQ ID NO: 160 - protein, 6 aa, Artificial Sequence
  Description of Artificial Sequence Synthetic 6xHis tag
HHHHHH

SEQ ID NO: 161 - protein, 10 aa, Artificial Sequence
  Description of Artificial Sequence Synthetic peptide
GGGGSGGGGS

SEQ ID NO: 162 - protein, 15 aa, Artificial Sequence
  Description of Artificial Sequence Synthetic peptide
EPKSCDKTHTCPPCP